(19) United States
(12) Patent Application Publication
(10) Pub. No.: US 2023/0357391 A1
(43) Pub. Date: Nov. 9, 2023

(54) CD47 BINDING AGENTS AND USES THEREOF

(71) Applicant: EXELIXIS, INC., Alameda, CA (US)

(72) Inventors: Bryan GLASER, Madison, WI (US); Bonnie HAMMER, Madison, WI (US); Seema KANTAK, Pacifica, CA (US)

(73) Assignee: EXELIXIS, INC., Alameda, CA (US)

(21) Appl. No.: 18/018,860

(22) PCT Filed: Aug. 3, 2021
(86) PCT No.: PCT/US2021/044310

§ 371 Date: Jan. 30, 2023

Related U.S. Application Data
(60) Provisional application No. 63/061,103, filed on Aug. 4, 2020.

(51) Int. Cl. C07K 16/28; C12N 15/63; A61K 45/06

(52) U.S. Cl. CPC C07K 16/2803; A61K 45/06; C12N 15/63; C07K 2317/55; C07K 2317/565; C07K 2317/92; C07K 2317/94

(57) ABSTRACT

The present disclosure provides CD47 binding agents (e.g., antibodies, including multispecific antibodies, such as bispecific antibodies) and uses thereof.

### CROSS-REFERENCE TO RELATED APPLICATIONS

This application claims the benefit of U.S. Provisional Application No. 63/061,103, filed Aug. 4, 2020, the disclosure of which is incorporated by reference herein in its entirety.

### SEQUENCE LISTING

This application incorporates by reference a Sequence Listing submitted with this application as a text file, entitled 14529-006-228_SEQ_LISTING.txt, created on Jul. 28, 2021, and is 53,848 bytes in size.

### FIELD

The present disclosure relates generally to binding agents, such as antibodies, that bind to CD47, including human CD47, and methods of their use.

### BACKGROUND

CD47 is a cell surface glycoprotein that functions as a regulator of phagocytosis mediated by cells of the innate immune system. CD47 interacts with multiple ligands, such as integrins, signal regulatory protein alpha (SlRPα), signal regulatory protein gamma (SlRPγ) and thrombospondins. CD47 inhibits phagocytosis by interacting with SIRPα on the surface of macrophages and dendritic cells, triggering a “don’t eat me” signal.

Expressing CD47 enables tumor cells to evade phagocytosis and escape from innate immune surveillance. Thus, CD47 has been a target for possible therapeutics. However, CD47 is broadly expressed on normal cells, such as hematopoietic cells, red blood cells (RBCs) and platelets. The broad expression of CD47 by healthy cells presents safety and efficacy challenges because targeting CD47 with a neutralizing antibody could affect healthy cells, possibly leading to toxic effects. Additionally, broad expresion of CD47 could also lead to a rapid elimination of CD47 binding agents, leading to poor pharmacokinetics and decreased efficacy.

Additionally, activation or loss of CD47 can result in enhanced proliferation in a cell type dependent mannor. For example, astrocytoma cells have been shown to have increased proliferation following activation of CD47 and TSP-1, whereas the normal astroglial cells have not. It has also been proposed that CD47 may facilitate proliferation of cancer cells through a P13K/Akt pathway.

Many of the anti-CD47 antibodies that have been reported are known to cause agglutination of RBCs upon blocking CD47 binding to SIRPα, which significantly lowers the therapeutic effect of such antibodies.

Accordingly, there remains a need in the art for agents that overcome these concerns and which target CD47 to treat, prevent, or alleviate phagocytic cell dysfunctional diseases, disorders, or conditions, including those involving tumor cells expressing CD47. The CD47 binding agents, compositions and methods provide herein satisfy this need and provide related advantages.

### SUMMARY

The present disclosure provides CD47 binding agents, including human CD47 binding agents. Such agents include antibodies that bind to CD47, for example, monospecific or multispecific (e.g., bispecific) antibodies that bind to CD47. Such antibodies, in some embodiments, compete for the binding of human CD47 with an antibody having a heavy chain variable region and a light chain variable region described herein (e.g., Table 1-3).

The present disclosure also provides compositions comprising a CD47 binding agent. Such compositions, in some embodiments, include antibodies that bind to CD47, for example, monospecific or multispecific (e.g., bispecific) antibodies that bind to CD47. Such compositions, in some embodiments, include antibodies that compete for the binding of human CD47 with an antibody having a heavy chain variable region and a light chain variable region described herein (e.g., Table 1-3).

The present disclosure also provides methods of treating, preventing, or alleviating a phagocytic cell dysfunctional disease, disorder, or condition, including one or more symptoms of the phagocytic cell dysfunctional disease, disorder, or condition with a CD47 binding agent or a composition comprising the agent, including a CD47 binding agent or composition comprising the agent. Such compositions include antibodies that bind to CD47, for example, monospecific or multispecific (e.g., bispecific) antibodies that bind to CD47.

### BRIEF DESCRIPTION OF THE DRAWINGS

FIGS. 1A-1C illustrate exemplary results from Octet binding assays, further described in Example 2.

FIGS. 2A-2D illustrate exemplary results from cell binding assays, further described in Example 3.

FIGS. 3A-3C illustrate exemplary results from CD47/SlRPα inhibiting assays, further described in Example 4.

FIG. 4 illustrates exemplary results from phagocytosis assays, further described in Example 5.

FIGS. 5A-5C illustrate exemplary results from SEC chromatography, further described in Example 7.

FIGS. 6A-6B illustrate exemplary results from HIC chromatography, further described in Example 7.

FIGS. 7A-7C illustrate exemplary results from SMAC chromatography, further described in Example 7.

FIGS. 8A-8B show a sequence alignment of heavy chain variable regions and light chain variable regions of C40, C56, and C59, including consensus sequences for VH CDR1, VH CDR2, VH CDR3, VL CDR1, VL CDR2, and VL CDR3. Boundaries of CDRs are indicated by Kabat, AbM, Chothia, Contact, and IMGT numbering.

### DETAILED DESCRIPTION

The present disclosure provides CD47 binding agents. Such agents include antibodies (e.g., monospecific or multispecific, including bispecific) that bind to CD47, including antibodies that bind to human CD47. Such binding agents are useful in compositions and in methods of treating, preventing, or alleviating a phagocytic cell dysfunctional disease, disorder, or condition, including one or more symptoms of the disease, disorder, or condition. Phagocytic cell dysfunctional diseases, disorders, and conditions include tumor immunity and associated cancers, including, but not limited to, any cancer wherein the tumor cells express or overexpress CD47. Such CD47 expressing tumor cells may help tumor cells escape immune surveillance and clearance (e.g., tumor immunity). In addition, CD47 binding agents described herein, such as CD47 binding antibodies (e.g., monospecific or multispecific antibodies, including bispecific antibodies), are useful to inhibit SIRPα signaling and/or enhance phagocytic cell function and thus enhance immune surveillance and removal of tumor cells. CD47 binding agents described herein, such as CD47 binding antibodies (e.g., monospecific or multispecific antibodies, including bispecific antibodies), are useful in compositions and in methods for enhancing phagocytic cell function, including the upregulation of cell-mediated immune responses.

The term “CD47,” “Cluster of Differentiation 47,” or “CD47 polypeptide” and similar terms refers to a polypeptide (“polypeptide” and “protein” are used interchangeably herein) or any native CD47 from any vertebrate source, including mammals such as primates (e.g., humans, cynomolgus monkey (cyno)), dogs, and rodents (e.g., mice and rats), unless otherwise indicated. CD47, also known in the art as integrin associated protein (IAP), has an extracellular N-terminal IgV domain, five transmembrane domains, and a short C-terminal intracellular tail. The term CD47 encompasses “full-length,” unprocessed CD47, as well as any form of CD47 or any fragment thereof that results from processing in the cell, including the four known alternatively spliced isoforms of CD47 that differ in the length of the intracellular tail. The term CD47 also encompasses naturally occurring variants of CD47, such as SNP variants, splice variants and allelic variants. CD47 is known in the art to interact with SIRPα and this interaction leads to cell signaling that includes, among other things, inhibition of phagocytosis by macrophages. The full-length amino acid sequence of human CD47 is provided below (exemplary extracellular domain = underline text):

MWPLVAALLLGSACCGSAQLLFNKTKSVEFTFCNDTVVIPCFVTNMEAQ NTTEVYVKWKFKGRDIYTFDGALNKSTVPTDFSSAKIEVSQLLKGDASLK MDKSDAVSHTGNYTCEVTELTREGETIIELKYRVVSWFSPNENILIVIFP
IFAILLFWGQFGIKTLKYRSGGMDEKTIALLVAGLVITVIVIVGAILFVP
GEYSLKNATGLGLIVTSTGILILLHYYVESTAIGLTSFVIAILVIQVIAY
ILAVVGLSLCIAACIPMHGPLLISGLSILALAQLLGLVYMKFVASNQKTI
QPPRKAVEEPLNAFKESKGMMNDE (SEQ ID NO:79).

Other related CD47 polypeptides that are also encompassed by the term CD47 include fragments, derivatives (e.g., substitution, deletion, truncations, and insertion variants), fusion polypeptides, and interspecies homologs that retain CD47 activity and/or are sufficient to generate an anti-CD47 immune response. As those skilled in the art will appreciate, a CD47 binding agent (e.g., an antibody) described herein can bind to a CD47 polypeptide, a CD47 polypeptide fragment, a CD47 antigen, and/or a CD47 epitope. An epitope may be part of a larger CD47 antigen, which may be part of a larger CD47 polypeptide fragment, which, in turn, may be part of a larger CD47 polypeptide. CD47 may exist in a native or denatured form. CD47 polypeptides described herein may be isolated from a variety of sources, such as from human tissue types or from another source, or prepared by recombinant or synthetic methods. A CD47 polypeptide may comprise a polypeptide having the same amino acid sequence as a corresponding CD47 polypeptide derived from nature. Orthologs to the CD47 polypeptide are also well known in the art.

The term “SlRPα,” “Signal-regulatory protein alpha,” or “Signal-regulatory protein α” and similar terms refers to a polypeptide (“polypeptide” and “protein” are used interchangeably herein) or any native SIRPα from any vertebrate source, including mammals such as primates (e.g., humans, cynomolgus monkey (cyno)), dogs, and rodents (e.g., mice and rats), unless otherwise indicated. SIRPα has an extracellular region, which includes three immunoglobulin superfamily domains - single V-set and two C1-set IgSF domains, a transmembrane domain and a cytoplasmic region containing an immunoreceptor tyrosine-based inhibition motif (ITIM). The term SIRPα also encompasses naturally occurring variants of SIRPα, such as SNP variants, splice variants and allelic variants. SIRPα is known in the art to interact with CD47, leading to phosphorization of the ITIM, which mediates its association with the phosphatase SH2-domain-containing protein tyrosine phosphatase \ (SHP2). The full-length amino acid sequence of human SIRPα is provided below:

MEPAGPAPGRLGPLLCLLLAASCAWSGVAGEEELQVIQPDKSVLVAAGET
ATLRCTATSLIPVGPIQWFRGAGPGRELIYNQKEGHFPRVTTVSDLTKRN
NMDFSIRIGNITPADAGTYYCVKFRKGSPDDVEFKSGAGTELSVRAKPSA
PVVSGPAARATPQHTVSFTCESHGFSPRDITLKWFKNGNELSDFQTNVDP
VGESVSYSIHSTAKVVLTREDVHSQVICEVAHVTLQGDPLRGTANLSETI
RVPPTLEVTQQPVRAENQVNVTCQVRKFYPQRLQLTWLENGNVSRTETAS
TVTENKDGTYNWMSWLLVNVSAHRDDVKLTCQVEHDGQPAVSKSHDLKVS
AHPKEQGSNTAAENTGSNERNIYIVVGVVCTLLVALLMAALYLVRIRQKK
AQGSTSSTRLHEPEKNAREITQDTNDITYADLNLPKGKKPAPQAAEPNNH
TEYASIQTSPQPASEDTLTYADLDMVHLNRTPKQPAPKPEPSFSEYASVQ
VPRK (SEQ ID NO:80).

As used herein, the term “binding agent” or a grammatical equivalent thereof refers to a molecule (e.g., antibody) with one or more antigen binding sites that binds an antigen. In some embodiments, a CD47 binding agent as described herein is an antibody, antibody fragment, or other peptide-based molecule that binds to CD47, such as human CD47.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. The present disclosure also includes antibody fragments (and/or polypeptides that comprise antibody fragments) that retain CD47 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled),. In general terms, a variable (V) region domain may be any suitable arrangement of immunoglobulin heavy (VH) and/or light (VL) chain variable domains. For example, the present disclosure also includes tetrameric antibodies comprising two heavy chain and two light chain molecules, an antibody light chain monomer, and an antibody heavy chain monomer. Thus, for example, the V region domain may be dimeric and contain VH-VH, VH-VL, or VL-VL dimers that bind CD47. If desired, the VH and VL chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). The binding agent, in some embodiments, contains a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

The term “monospecific” when used in reference to a binding agent (e.g., an antibody) as used herein denotes a binding agent that has one or more binding sites each of which bind to the same epitope of the same antigen.

The term “bispecific” when used in reference to a binding agent (e.g., an antibody) means that the binding agent is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific binding agent (e.g., an antibody) may have a 1+1 format. Other bispecific binding agent (e.g., an antibody) formats may be 2+1 or 1+2 formats (comprising two binding sites for a first antigen or epitope and one binding site for a second antigen or epitope) or 2+2 formats (comprising two binding sites for a first antigen or epitope and two binding sites for a second antigen or epitope). When a bispecific binding agent (e.g., an antibody) comprises two antigen binding sites, each may bind to a different antigenic determinant. Such a bispecific binding agent (e.g., an antibody) may bind to two different epitopes on the same antigen (e.g., epitopes on CD47).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The terms “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component. Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains.

As used herein, an “antigen” is a moiety or molecule that contains an epitope to which a binding agent (e.g., an antibody) can bind. As such, an antigen can be bound by an antibody. In some embodiments, the antigen, to which a binding agent (e.g., an antibody) described herein binds, is CD47 (e.g., human CD47), or a fragment thereof.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human CD47. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other non-CD47 proteins. In some embodiments “specifically binds” means, for instance, that a polypeptide or molecule binds a protein or target with a KD of about 0.1 mM or less, but more usually less than about 1 µM. In some embodiments, “specifically binds” means that a polypeptide or molecule binds a target with a KD of at least about 0.1 µM or less, at least about 0.01 µM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 sytem (ForteBio, Menlo Park, CA). Alternatively, the K_{D} may be also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by Biacore, using, for example, a BIAcoreTM-2000 or a BIAcoreTM-3000 BIAcore, Inc., Piscataway, NJ). An “on-rate” or “rate of association” or “association rate” or “kₒₙ,” as well as an “off-rate” or “rate of dissociation” or “dissociation rate” or “k_{off},” may can also be determined with the same SPR or BLI techniques described above using, for example, the OctetQK384 sytem (ForteBio, Menlo Park, CA) or a BIAcoreTM-2000 or a BIAcoreTM-3000 (BIAcore, Inc., Piscataway, NJ), respectively.

The term “compete” when used in the context of CD47 binding agents (e.g., antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen binding protein, such as a reference antibody) to a common antigen (e.g., CD47). Numerous types of competitive binding assays can be used to determine if a test binding agent competes with a reference molecule for binding to CD47 (e.g., human CD47). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619) solid phase direct labeled assay, solid phase direct labeled sandwich assay (see, e.g., Harlow and Lane, (1988) Antibodies, A Laboratory Manual, Cold Spring Harbor Press); solid phase direct label RIA using 1-125 label (see, e.g., Morel et al., (1988) Molec. Immunol. 25:7-15); solid phase direct biotin-avidin EIA (see, e.g., Cheung, et al., (1990) Virology 176:546-552); and direct labeled RIA (Moldenhauer et al., (1990) Scand. J. Immunol. 32:77-82). Typically, such an assay involves the use of a purified antigen (e.g., CD47, such as human CD47) bound to a solid surface or cells bearing either of an unlabelled test antigen binding protein (e.g., test CD47 antibody) or a labeled reference antigen binding protein (e.g., reference CD47 antibody). Competitive inhibition may be measured by determining the amount of label bound to the solid surface or cells in the presence of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Additional details regarding methods for determining competitive binding are described herein, as shown in Example 8. Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96% or 97%, 98%, 99% or more.

As used herein, the term “constant region” or “constant domain” is a well-known antibody term of art and refers to an antibody portion, e.g., for example, a carboxyl terminal portion of a light and/or heavy chain which is not directly involved in binding of an antibody to antigen but which can exhibit various effector functions, such as interaction with the Fc receptor. The term include the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. An exemplary Fc region sequence is provided below (CH2 domain = bold text; CH3 domain = underline text):

CPPCPAPELLGGPSVFLFPPKPKDTLMISRTPEVTCVVVDVSHEDPEVK FNWYVDGVEVHNAKTKPREEQYNSTYRVVSVLTVLHQDWLNGKEYKCKVS NKALPAPIEKTISKAKGQPREPQVYTLPPSRDELTKNQVSLTCLVKGFYP SDIAVEWESNGQPENNYKTTPPVLDSDGSFFLYSKLTVDKSRWQQGNVFS CSVMHEALHNHYTQKSLSLSPGK (SEQ IDNO:81).

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, (e.g., substituting, addition, or deletion) preferably one or more amino acid substitution(s). In some embodicments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein may have a loss of effctor function (e.g., silent Fc). An exemplary variant Fc region (“silent Fc”) sequence is provided below (CH2 domain = bold text with amino acid changes underlined; CH3 domain = underline text):

CPPCPAPEAAGGPSVFLFPPKPKDTLMISRTPEVTCVVVDVSHEDPEVKF NWYVDGVEVHNAKTKPREEQYNSTYRVVSVLTVLHQDWLNGKEYKCKVSN KALKAPIEKTISKAKGQPREPQVYTLPPSRDELTKNQVSLTCLVKGFYPS DIAVEWESNGQPENNYKTTPPVLDSDGSFFLYSKLTVDKSRWQQGNVFSC SVMHEALHNHYTQKSLSLSPGK (SEQ IDNO:82).

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (µ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (ĸ) of lambda (λ) based on the amino acid sequence of the constant domains. Light chain amino acid sequences are well known in the art.

The terms “antigen binding fragment,” “antigen binding domain,” “antigen binding region,” and similar terms refer to that portion of an antibody, which comprises the amino acid residues that interact with an antigen and confer on the binding fragment, domain, or region its specificity and affinity for the antigen (e.g., the CDRs). “Antigen binding fragment” as used herein include “antibody fragment,” which comprise a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen binding sites that bind to a CD47 antigen.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In some embodiments, an antibody is a 4-chain antibody unit comprising two heavy (H) chain / light (L) chain pairs, wherein the amino acid sequences of the H chains are identical and the amino acid sequences of the L chains are identical. In some embodiments, the H and L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprise a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to CD47, such as human CD47, meaning that the antibody or fragment thereof binds CD47 with greater affinity than it binds to an unrelated control protein and/or binds human CD47 with greater affinity than it binds to an unrelated control protein. For example, the antibody or fragment thereof may specifically recognize and bind CD47 or a portion thereof. “Specific binding” means that the antibody or fragment thereof binds to CD47 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind CD47 substantially exclusively (e.g., is able to distinguish CD47 from other known polypeptides, for example, by virtue of measurable differences in binding affinity). In some embodiments, a CD47 binding agent (e.g., an antibody) may react with CD47 sequences other than human CD47 sequences (e.g., cynomolgous CD47 sequences).

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four FRs (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular’s AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT) Information System® (Lefranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 52a/53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

The term “vector” refers to a substance that is used to carry or include a nucleic acid sequences, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell’s chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a CD47 binding agent as described herein), and it is further understood that expression levels can be optimized to obtain sufficient expression using methods well known in the art.

A “phagocytic cell dysfunctional disease” and “phagocytic cell dysfunctional disorder” and “phagocytic cell dysfunctional condition” are used interchangeably and refer to any disease, disorder or condition that is completely or partially caused by or is the result of CD47 or the interaction of CD47 with SIRPα and/or alternatively any disease, disorder, or condition in which it is desirable to inhibit the in vivo effects of the interaction of CD47 with SIRPα. A phagocytic cell dysfunctional disease includes a disease, disorder or condition that is characterized by or associated with decreased phagocytic activity of immune cells (e.g., neutrophils, macrophages, dendritic cells, B lymphocytes). In some embodiments, a phagocytic cell dysfunctional disease is a disease, disorder or contition that is specifically associated with inappropriate increased signaling through SIRPα. In some embodiments, a phagocytic cell dysfunctional disease is one in which phagocytic cells (e.g., macrophages) have decreased ability to ingest or engulf other cells (e.g., a tumor cell) or particles. In some embodiments, the decreased ability to ingest or engulf other cells or particles results in ineffective control of a pathogen or tumor, including but not limited to tumors expressing CD47. Examples of a phagocytic cell dysfunctional disease characterized by phagocytic cell dysfunction include unresolved acute infection, chronic infection and tumor immunity (e.g., from any cancers, including but not limited to cancers that express or overexpress CD47).

“Tumor immunity” refers to the process in which tumors evade immune recognition and clearance. Thus, as a therapeutic concept, tumor immunity is “treated” when such evasion is attenuated and the tumors are recognized and attacked by the immune system. Such treatment thus includes treatment of any cancer. Examples of tumor recognition include tumor binding, tumor strinkage and tumor clearance.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an antibody described herein). A “therapeutically effective amount” of a substance/molecule/agent of the present disclosure (e.g., a CD47 antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s). The full therapeutic or prophylactic effect does not necessarily occur by administration of one dose, and may occur only after administration of a series of doses. Thus, a therapeutically or prophylactically effective amount may be administered in one or more administrations.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight ((e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONlCS™. The term “carrier” can also refer to a diluent, adjuvant (e.g., Freund’s adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington’s Pharmaceutical Sciences (1990) Mack Publishing Co., Easton, PA. Compositions, including pharmaceutical compounds, may contain a prophylactically or therapeutically effective amount of a CD47 binding agent (e.g., an antibody), for example, in isolated or purified form, together with a suitable amount of carrier so as to provide the form for proper administration to the subject (e.g., patient). The formulation should suit the mode of administration.

In some embodiments, the present disclosure provides CD47 binding agents that can be used herein as therapeutic agents. Such agents include antibodies (e.g., monospecific or multispecific, including bispecific) that bind to CD47. Exemplary antibodies include polyclonal, monoclonal, humanized, human, bispecific, and heteroconjugate antibodies, as well as variants thereof having increased or decreased affinity or other properties.

In some embodiments, described herein are CD47 binding agents (e.g., antibodies) that bind to CD47, including a CD47 polypeptide, a CD47 polypeptide fragment, a CD47 peptide or a CD47 epitope. In some embodiments, the CD47 binding agents are human or humanized antibodies (e.g., comprising human constant regions) that bind CD47, including a CD47 polypeptide, a CD47 polypeptide fragment, a CD47 peptide or a CD27 epitope. In some embodiments, a CD47 binding agent (e.g., an antibody), such as a human CD47 binding agent, can bind to CD47 expressed on the surface of a mammalian (e.g., human) cell, including a CD47 expressing tumor cell. In some embodiments, a CD47 binding agent (e.g., an antibody) binds a CD47 extracellular epitope exposed on a cell such as a tumor cell (e.g., a CD47 epitope). In some embodiments, described herein is a CD47 binding agent (e.g., an antibody) that binds to CD47, such as human CD47 or portions thereof. In some embodiments, CD47 is a human CD47. In some embodiments, a CD47 binding agent is a human CD47 binding agent (e.g., an antibody that binds to human CD47). An exemplary amino acid sequence of human CD47 is described herein.

In some embodiments, the CD47 binding agents (e.g., antibodies) described herein compete for the binding to CD47, such as human CD47, with a CD47 binding agent (e.g., an antibody) that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-3. Accordingly, in some embodiments, a CD47 binding agent (e.g., an antibody) described herein competes for the binding to CD47, such as human CD47, with a CD47 binding agent (e.g., an antibody) that comprises one, two, and/or three VH CDRs and/or one, two, and/or three VL CDRs from: (a) the antibody designated C40; (b) the antibody designated C56; or (c) the antibody designated C59, as shown in Tables 1-3. In some embodiments, a CD47 binding agent (e.g., an antibody) described herein competes for the binding to CD47, such as human CD47, with a CD47 binding agent (e.g., an antibody) that comprises one, two, and/or three VH CDRs and one, two, and/or three VL CDRs from: (a) the antibody designated C40; (b) the antibody designated C56; or (c) the antibody designated C59, as shown in Tables 1-3. In some embodiments, a CD47 binding agent (e.g., an antibody) described herein competes for the binding to CD47, such as human CD47, with a CD47 binding agent (e.g., an antibody) that comprises a VH region and VL region from: (a) the antibody designated C40; (b) the antibody designated C56; or (c) the antibody designated C59, as shown in Tables 1-3. In some embodiments, a CD47 binding agent (e.g., an antibody) described herein competes for the binding to CD47, such as human CD47, with a CD47 binding agent (e.g., an antibody) that comprises: (a) a VH region comprising the amino acid sequence of SEQ ID NO:25 and a VL region comprising the amino acid sequence of SEQ ID NO:26; (b) a VH region comprising the amino acid sequence of SEQ ID NO:51 and a VL region comprising the amino acid sequence of SEQ ID NO:52; or (c) a VH region comprising the amino acid sequence of SEQ ID NO:77 and a VL region comprising the amino acid sequence of SEQ ID NO:78.

In some embodiments, the CD47 binding agents (e.g., antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 depicted in Tables 1-3. Accordingly, in some embodiments, a CD47 binding agent (e.g., an antibody) described herein comprises one, two, and/or three heavy chain CDRs and/or one, two, and/or three light chain CDRs from: (a) the antibody designated C40; (b) the antibody designated C56; or (c) the antibody designated C59, as shown in Tables 1-3. In some embodiments, a CD47 binding agent (e.g., an antibody) described herein comprises one, two, and/or three heavy chain CDRs and one, two, and/or three light chain CDRs from: (a) the antibody designated C40; (b) the antibody designated C56; or (c) the antibody designated C59, as shown in Tables 1-3.

In some embodiments, a CD47 binding agent (e.g., an antibody) comprises a VH region, which comprises VH CDR1, VH CDR2, and/or VH CDR3, and a VL region, which comprises VL CDR1, VL CDR2, and/or VL CDR3, of any one of the binding agents described herein (see, e.g., Table 1, Table 2, Table 3). Accordingly, in some embodiments, a CD47 binding agent (e.g., an antibody) described herein comprises one, two, and/or three heavy chain CDRs and/or one, two, and/or three light chain CDRs from Table 1. In some embodiments, a CD47 binding agent described herein comprises one, two, and/or three heavy chain CDRs and/or one, two, and/or three light chain CDRs from Table 2. In some embodiments, a CD47 binding agent (e.g., an antibody) described herein comprises one, two, and/or three heavy chain CDRs and/or one, two, and/or three light chain CDRs from Table 3. In some embodiments, a CD47 binding agent (e.g., an antibody) described herein is bispecific and comprises a first binding domain that comprises one, two, and/or three heavy chain CDRs and/or one, two, and/or three light chain CDRs from Table 1, Table 2, or Table 3 and a second binding domain that comprises one, two, and/or three heavy chain CDRs and/or one, two, and/or three light chain CDRs from a binding agent that binds to a second target antigen that is not CD47.

The antibody designated C40 comprises a VH sequence that is SEQ ID NO:25 and a VL sequence that is SEQ ID NO:26.

The antibody designated C56 comprises a VH sequence that is SEQ ID NO:51 and a VL sequence that is SEQ ID NO:52.

The antibody designated C59 comprises a VH sequence that is SEQ ID NO:77 and a VL sequence that is SEQ ID NO:78.

TABLE 1

Antibody C40

| | | Exemplary | IMGT | Kabat | Chothia | Contact | AbM |
|---|---|---|---|---|---|---|---|
| VH CDR Seq. | VH CDR1 | GFTFSYYYIH (SEQ ID NO:1) | GFTFSYYY (SEQ ID NO:7) | YYYIH (SEQ ID NO:12) | GFTFSYY (SEQ ID NO:13) | SYYYIH (SEQ ID NO: 18) | GFTFSYYYIH (SEQ ID NO:1) |
| | VH CDR2 | WIDPYGHSTTY ADSVKG (SEQ ID NO:2) | IDPYGHST (SEQ ID NO:8) | WIDPYGHSTTY ADSVKG (SEQ ID NO:2) | PYGH (SEQ ID NO:14) | WVAWIDPYGHS TT (SEQ ID NO:19) | WIDPYGHSTT (SEQ ID NO:24) |
| | VH CDR3 | GGRGAMDY (SEQ ID NO:3) | ARGGRGAMDY (SEQ ID NO:9) | GGRGAMDY (SEQ ID NO:3) | GRGAMD (SEQ ID NO:15) | ARGGRGAMD (SEQ ID NO:20) | GGRGAMDY (SEQ ID NO:3) |
| VL CDR Seq. | VL CDR1 | RASQSVSSAVA (SEQ ID NO:4) | QSVSSA (SEQ ID NO:10) | RASQSVSSAVA (SEQ ID NO:4) | SQSVSSA (SEQ ID NO:16) | SSAVAWY (SEQ ID NO:21) | RASQSVSSAVA (SEQ ID NO:4) |
| | VL CDR2 | SASSLYS (SEQ ID NO:5) | SAS (SEQ ID NO:11) | SASSLYS (SEQ ID NO:5) | SAS (SEQ ID NO:11) | LLIYSASSLY (SEQ ID NO:22) | SASSLYS (SEQ ID NO:5) |
| | VL CDR3 | QQRYSSLLT (SEQ ID NO:6) | QQRYSSLLT (SEQ ID NO:6) | QQRYSSLLT (SEQ ID NO:6) | RYSSLL (SEQ ID NO:17) | QQRYSSLL (SEQ ID NO:23) | QQRYSSLLT (SEQ ID NO:6) |

VH Sequence: EVQLVESGGGLVQPGGSLRLSCAASGFTFSYYYIHWVRQAPGKGLEVWAWIDPYGHSTTYADSVKGRFTISADTSKNTAYLQMNSLRAE DTAVYYCARGGRGAMDYWGQGTLVT (SEQ ID NO:25)
VL Sequence: DIQMTQSPSSLSASVGDRVTITCRASQSVSSAVAWYQQKPGKAPKLLIYSASSLYSGVPSRFSGSRSGTDFTLTISSLQPEDFATYYCQQR YSSLLTFGQGTKVEIK (SEQ ID NO:26)

TABLE 2

Antibody C56

| | | Exemplary | IMGT | Kabat | Chothia | Contact | AbM |
|---|---|---|---|---|---|---|---|
| VH CDR Seq. | VH CDR1 | GFTFTYYYIH (SEQ ID NO:27) | GFTFTYYY (SEQ ID NO:33) | YYYIH (SEQ ID NO:38) | GFTFTYY (SEQ ID NO:39) | TYVYIH (SEQ ID NO:44) | GFTFTYYYIH (SEQ ID NO:27) |
| | VH CDR2 | FIDPYSGSTEYA DSVKG (SEQ ID NO:28) | IDPYSGST (SEQ ID NO:34) | FIDPYSGSTEYA DSVKG (SEQ ID NO:28) | PYSG (SEQ ID NO:40) | WVAFIDPYSGS TE (SEQ ID NO:45) | FIDPYSGSTE (SEQ ID NO:50) |
| | VH CDR3 | GGLYALDY (SEQ ID NO:29) | ARGGLYALDY (SEQ ID NO:35) | GGLYALDY (SEQ ID NO:29) | GLYALD (SEQ ID NO:41) | ARGGLYALD (SEQ ID NO:46) | GGLYALDY (SEQ ID NO:29) |
| VL CDR Seq. | VL CDR1 | RASQSVSSAVA (SEQ ID NO:30) | QSVSSA (SEQ ID NO:36) | RASQSVSSAVA (SEQ ID NO:30) | SQSVSSA (SEQ ID NO:42) | SSAVAWY (SEQ ID NO:47) | RASQSVSSAVA (SEQ ID NO:30) |
| | VL CDR2 | SASSLYS (SEQ ID NO:31) | SAS (SEQ ID NO:37) | SASSLYS (SEQ ID NO:31) | SAS (SEQ ID NO:37) | LLIYSASSLY (SEQ ID NO:48) | SASSLYS (SEQ ID NO:31) |
| | VL CDR3 | QQGRSDLRT (SEQ ID NO:32) | QQGRSDLRT (SEQ ID NO:32) | QQGRSDLRT (SEQ ID NO:32) | GRSDLR (SEQ ID NO:43) | QQGRSDLR (SEQ ID NO:49) | QQGRSDLRT (SEQ ID NO:32) |

VH Sequence: EVQLVESGGGLVQPGGSLRLSCAASGFTFTYYYIHWVRQAPGKGLEWVAFIDPYSGSTEYADSVKGRFTISADTSKNTAYLQMNSLRAE DTAVYYCARGGLYALDYWGQGTLVT (SEQ ID NO:51)
VL Sequence: DIQMTQSPSSLSASVGDRVTITCRASQSVSSAVAWYQQKPGKAPKLLIYSASSLYSGVPSRFSGSRSGTDFTLTISSLQPEDFATYYCQQ GRSDLRTFGQGTKVEIK (SEQ ID NO:52)

TABLE 3

Antibody C59

| | | Exemplary | IMGT | Kabat | Chothia | Contact | AbM |
|---|---|---|---|---|---|---|---|
| VH CDR Seq. | VH CDR1 | GFTFTSYYIH (SEQ ID NO:53) | GFTFTSYY (SEQ ID NO:59) | SYYIH (SEQ ID NO:64) | GFTFTSY (SEQ ID NO:65) | TSYYIH (SEQ ID NO:70) | GFTFTSYYIH (SEQ ID NO:53) |
| | VH CDR2 | YIDSKHGTTQYA DSVKG (SEQ ID NO:54) | IDSKHGTT (SEQ ID NO:60) | YIDSKHGTTQYA DSVKG (SEQ ID NO:54) | SKHG (SEQ ID NO:66) | WVAYIDSKHGT TQ (SEQ ID NO:71) | YIDSKHGTTQ (SEQ ID NO:76) |
| | VH CDR3 | GGRSAMDY (SEQ ID NO:55) | ARGGRSAMDY (SEQ ID NO:61) | GGRSAMDY (SEQ ID NO:55) | GRSAMD (SEQ ID NO:67) | ARGGRSAMD (SEQ ID NO:72) | GGRSAMDY (SEQ ID NO:55) |
| VL CDR Seq. | VL CDR1 | RASQSVSSAVA (SEQ ID NO:56) | QSVSSA (SEQ ID NO:62) | RASQSVSSAVA (SEQ ID NO:56) | SQSVSSA (SEQ ID NO:68) | SSAVAWY (SEQ ID NO:73) | RASQSVSSAVA (SEQ ID NO:56) |
| | VL CDR2 | SASSLYS (SEQ ID NO:57) | SAS (SEQ ID NO:63) | SASSLYS (SEQ ID NO:57) | SAS (SEQ ID NO:63) | LLIYSASSLY (SEQ ID NO:74) | SASSLYS (SEQ ID NO:57) |
| | VL CDR3 | QQRTTSLLT (SEQ ID NO:58) | QQRTTSLLT (SEQ ID NO:58) | QQRTTSLLT (SEQ ID NO:58) | RTTSLL (SEQ ID NO:69) | QQRTTSLL (SEQ ID NO:75) | QQRTTSLLT (SEQ ID NO:58) |

VH Sequence: EVQLVESGGGLVQPGGSLRLSCAASGFTFTSYYIHWVRQAPGKGLEWVAYIDSKHGTTQYADSVKGRFTISADTSKNTAYLQMNSLRAED TAVYYCARGGRSAMDYWGQGTLVT (SEQ ID NO:77)
VL Sequence: DIQMTQSPSSLSASVGDRVTITCRASQSVSSAVAWYQQKPGKAPKLLIYSASSLYSGVPSRFSGSRSGTDFTLTISSLQPEDFATYYCQQR TTSLLTFGQGTKVEIK (SEQ ID NO:78)

In some embodiments, CD47 binding agents (e.g., antibodies, such as bispecific antibodies), including human CD47 binding agents, described herein comprise a VH region or VH domain. In other embodiments, CD47 binding agents (e.g., antibodies, such as bispecific antibodies), including human CD47 binding agents, described herein comprise a VL region or VL domain. In some embodiments, CD47 binding agents (e.g., antibodies, such as bispecific antibodies), including human CD47 binding agents, described herein have a combination of (i) a VH domain or VH region; and/or (ii) a VL domain or VL region.

In some embodiments, CD47 binding agents (e.g., antibodies, such as bispecific antibodies), including human CD47 binding agents, described herein comprise heavy chain having a combination of (i) a VH domain described in any one of Tables 1-3; and (ii) one or more heavy chain constant domains (e.g., CH1, Hinge, CH2, and CH3). An exemplary IgG heavy chain comprises any VH domain as described herein and the following CH1, Hinge, CH2, and CH3 amino acid sequence:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO:96).

Another exemplary IgG heavy chain comprises any VH domain as described herein and the following CH1, Hinge, CH2, and CH3 amino acid sequence:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALKAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO:98)

In some embodiments, CD47 binding agents (e.g., antibodies, such as bispecific antibodies), including human CD47 binding agents, described herein comprise a light chain having a combination of (i) a VL domain described in any one of Tables 1-3; and (ii) a light chain constant domain (CL). An exemplary light chain (e.g., for pairing with an IgG heavy chain) comprises any VL domain as described herein and the following CL amino acid sequence:

RTVAAPSVFIFPPSDSQLKSGTASVVCLLNNFYPREAKVQWKVDNALQSG
NSQESVTEQDSKDSTYSLSSTLTLSKADYEKHKVYACEVTHQGLSSPVTK
SFNRGEC (SEQ IDNO:97).

In some embodiments, CD47 binding agents (e.g., antibodies, such as bispecific antibodies), including human CD47 binding agents, described herein comprise (a) a heavy chain having a combination of (i) a VH domain described in any one of Tables 1-3, and (ii) one or more heavy chain constant domains (e.g., CH1, Hinge, CH2, and CH3); and (b) a light chain having a combination of (i) a VL domain described in any one of Tables 1-3, and (ii) a light chain constant domain (CL). An exemplary CD47 binding agent (e.g., an antibody) comprises an IgG heavy chain comprising any VH domain as described herein and the amino acid sequence of SEQ ID NO:96 or 98, and a light chain comprising any VL domain as described herein and the amino acid sequence of SEQ ID NO:97.

In some embodiments, CD47 binding agents (e.g., antibodies, such as bispecific antibodies), including a human CD47 binding agent, described herein comprises one or more CDRs, including six CDRs, for example, VH CDR1, VH CDR2, VH CDR3, VL CDR1, VL CDR2, and/or VL CDR3 identified in Table 1. In some embodiments, CD47 binding agents (e.g., antibodies, such as bispecific antibodies), including a human CD47 binding agent, described herein comprises one or more, including six CDRs, for example, VH CDR1, VH CDR2, VH CDR3, VL CDR1, VL CDR2, and/or VL CDR3 identified in Table 2. In some embodiments, CD47 binding agents (e.g., antibodies, such as bispecific antibodies), including a human CD47 binding agent, described herein comprises one or more, including six CDRs, for example, VH CDR1, VH CDR2, VH CDR3, VL CDR1, VL CDR2, and/or VL CDR3 identified in Table 3. In some embodiments, CD47 binding agents (e.g., antibodies, such as bispecific antibodies), including a human CD47 binding agent, described herein comprises one or more, including six CDRs, for example, VH CDR1, VH CDR2, VH CDR3, VL CDR1, VL CDR2, and/or VL CDR3 identified in Tables 1, 2 and/or 3.

In some embodiments, CD47 binding agents (e.g., antibodies, such as bispecific antibodies), including human CD47 binding agents, described herein comprise one or more CDRs, including three VH CDRs, for example, VH CDR1, VH CDR2, VH CDR3, listed in Table 1. In other embodiments, CD47 binding agents (e.g., antibodies, such as bispecific antibodies), including human CD47 binding agents, described herein comprise one or more CDRs, including three CDRs, for example, VL CDR1, VL CDR2, and/or VL CDR3, listed in Table 1. In yet other embodiments, CD47 binding agents (e.g., antibodies, such as bispecific antibodies), including human CD47 binding agents, described herein comprise one or more CDRs, including three VH CDRs, for example, VH CDR1, VH CDR2, VH CDR3, listed in Table 1 and one or more CDRs, including three VL CDRs, for example, VL CDR1, VL CDR2, and/or VL CDR3, listed in Table 1.

In some embodiments, a CD47 binding agent (e.g., an antibody, such as a bispecific antibody) described herein comprises one or more complementarity determining regions (CDRs) comprising an amino acid sequence selected from a group consisting of SEQ ID NOS: 1-24, 27-50, and 53-76. In some embodiments, a CD47 binding agent (e.g., an antibody, such as a bispecific antibody) described herein comprises two or more complementarity determining regions (CDRs) comprising an amino acid sequence selected from a group consisting of SEQ ID NOS: 1-24, 27-50, and 53-76. In some embodiments, a CD47 binding agent (e.g., an antibody, such as a bispecific antibody) described herein comprises three or more complementarity determining regions (CDRs) comprising an amino acid sequence selected from a group consisting of SEQ ID NOS: 1-24, 27-50, and 53-76. In some embodiments, a CD47 binding agent (e.g., an antibody, such as a bispecific antibody) described herein comprises four or more complementarity determining regions (CDRs) comprising an amino acid sequence selected from a group consisting of SEQ ID NOS: 1-24, 27-50, and 53-76. In some embodiments, a CD47 binding agent (e.g., an antibody, such as a bispecific antibody) described herein comprises five or more complementarity determining regions (CDRs) comprising an amino acid sequence selected from a group consisting of SEQ ID NOS: 1-24, 27-50, 53-76. In some embodiments, a CD47 binding agent (e.g., an antibody, such as a bispecific antibody) described herein comprises six or more complementarity determining regions (CDRs) comprising an amino acid sequence selected from a group consisting of SEQ ID NOS: 1-24, 27-50, 53-76.

In some embodiments, a CD47 binding agent (e.g., an antibody, such as a bispecific antibody) described herein comprise one or more (e.g., one, two or three) VH CDRs listed in Tables 1-3. In other embodiments, a CD47 binding agent (e.g., an antibody, such as a bispecific antibody) described herein comprises one or more (e.g., one, two or three) VL CDRs listed in Tables 1-3. In yet other embodiments, a CD47 binding agent (e.g., an antibody, such as a bispecific antibody) described herein comprises one or more (e.g., one, two or three) VH CDRs listed in Tables 1-3 and one or more VL CDRs listed in Tables 1-3. Accordingly, in some embodiments, a CD47 binding agent (e.g., an antibody, such as a bispecific antibody) described herein comprises a VH CDR1 having the amino acid sequence of any one of SEQ ID NOS: 1, 7, 12, 13, 18, 27, 33, 38, 39, 44, 53, 59, 64, 65, and 70. In some embodiments, a CD47 binding agent (e.g., an antibody, such as a bispecific antibody) described herein comprises a VH CDR2 having the amino acid sequence of any one of SEQ ID NOS: 2, 8, 14, 19, 24, 28, 34, 40, 45, 50, 54, 60, 66, 71, and 76. In some embodiments, a CD47 binding agent (e.g., an antibody, such as a bispecific antibody) described herein comprises a VH CDR3 having the amino acid sequence of any one of SEQ ID NOS: 3, 9, 15, 20, 29, 35, 41, 46, 55, 61, 67, and 72. In some embodiments, a CD47 binding agent (e.g., an antibody, such as a bispecific antibody) described herein comprises a VH CDR1 and/or a VH CDR2 and/or a VH CDR3 independently selected from a VH CDR1, VH CDR2, VH CDR3 as depicted in any one of the amino acid sequences depicted in Table 1-3. In some embodiments, a CD47 binding agent (e.g., an antibody, such as a bispecific antibody) described herein comprises a VL CDR1 having the amino acid sequence of any one of SEQ ID NOS: 4, 10, 16, 21, 30, 36, 42, 47, 56, 62, 68, and 73. In another embodiment, a CD47 binding agent (e.g., an antibody, such as a bispecific antibody) described herein comprises a VL CDR2 having the amino acid sequence of any one of SEQ ID NOS: 5, 11, 22, 31, 37, 48, 57, 63, and 74. In some embodiments, a CD47 binding agent (e.g., an antibody, such as a bispecific antibody) described herein comprises a VL CDR3 having the amino acid sequence of any one of SEQ ID NOS: 6, 17, 23, 32, 43, 49, 58, 69, and 75. In some embodiments, a CD47 binding agent (e.g., an antibody, such as a bispecific antibody) described herein comprises a VL CDR1 and/or a VL CDR2 and/or a VL CDR3 independently selected from a VL CDR1, VL CDR2, VL CDR3 as depicted in any one of the amino acid sequences depicted in Tables 1-3.

In some embodiments, a CD47 binding agent (e.g., an antibody, such as a bispecific antibody) described herein comprises a heavy chain variable (VH) region comprising: (1) a VH CDR1 having an amino acid sequence of selected from the group consisting of: (i) SEQ ID NO:1, 27, or 53, (ii) SEQ ID NO:7, 33, or 59, (iii) SEQ ID NO:12, 38, or 64, (iv) SEQ ID NO:13, 39, or 65, and (v) SEQ ID NO:18, 44, or 70; (2) a VH CDR2 having an amino acid sequence of selected from the group consisting of: (i) SEQ ID NO:2, 28, or 54, (ii) SEQ ID NO:8, 34, or 60, (iii) SEQ ID NO:14, 40, or 66, (iv) SEQ ID NO:19, 45, or 71, and (v) SEQ ID NO:24, 50, or 76; and (3) a VH CDR3 having an amino acid sequence of selected from the group consisting of: (i) SEQ ID NO:3, 29, or 55, (ii) SEQ ID NO:9, 35, or 61, (iii) SEQ ID NO:15, 41, or 67, and (iv) SEQ ID NO:20, 46, or 72; and/or a light chain variable (VL) region comprising: (1) a VL CDR1 having an amino acid sequence of selected from the group consisting of: (i) SEQ ID NO:4, 30, or 56, (ii) SEQ ID NO:10, 36, or 62, (iii) SEQ ID NO:16, 42, or 68, and (iv) SEQ ID NO:21, 47, or 73; (2) a VL CDR2 having an amino acid sequence of selected from the group consisting of: (i) SEQ ID NO:5, 31, or 57, (ii) SEQ ID NO:11, 37, or 63, and (iii) SEQ ID NO:22, 48, or 74; and (3) a VL CDR3 having an amino acid sequence of selected from the group consisting of: (i) SEQ ID NO:6, 32, or 58, (ii) SEQ ID NO:17, 43, or 69, and (iii) SEQ ID NO:23, 49, or 75.

In some embodiments, a CD47 binding agent (e.g., an antibody, such as a bispecific antibody) described herein comprises a heavy chain variable (VH) region comprising: (1) a VH CDR1 having an amino acid sequence of selected from the group consisting of: (i) SEQ ID NO:1, 27, or 53, (ii) SEQ ID NO:7, 33, or 59, (iii) SEQ ID NO:12, 38, or 64, (iv) SEQ ID NO:13, 39, or 65, and (v) SEQ ID NO:18, 44, or 70; (2) a VH CDR2 having an amino acid sequence of selected from the group consisting of: (i) SEQ ID NO:2, 28, or 54, (ii) SEQ ID NO:8, 34, or 60, (iii) SEQ ID NO:14, 40, or 66, (iv) SEQ ID NO:19, 45, or 71, and (v) SEQ ID NO:24, 50, or 76; and (3) a VH CDR3 having an amino acid sequence of selected from the group consisting of: (i) SEQ ID NO:3, 29, or 55, (ii) SEQ ID NO:9, 35, or 61, (iii) SEQ ID NO:15, 41, or 67, and (iv) SEQ ID NO:20, 46, or 72.

In some embodiments, a CD47 binding agent (e.g., an antibody, such as a bispecific antibody) described herein comprises a light chain variable (VL) region comprising: (1) a VL CDR1 having an amino acid sequence of selected from the group consisting of: (i) SEQ ID NO:4, 30, or 56, (ii) SEQ ID NO:10, 36, or 62, (iii) SEQ ID NO:16, 42, or 68, and (iv) SEQ ID NO:21, 47, or 73; (2) a VL CDR2 having an amino acid sequence of selected from the group consisting of: (i) SEQ ID NO:5, 31, or 57, (ii) SEQ ID NO:11, 37, or 63, and (iii) SEQ ID NO:22, 48, or 74; and (3) a VL CDR3 having an amino acid sequence of selected from the group consisting of: (i) SEQ ID NO:6, 32, or 58, (ii) SEQ ID NO:17, 43, or 69, and (iii) SEQ ID NO:23, 49, or 75.

Also described herein are CD47 binding agents (e.g., antibodies, such as bispecific antibodies) comprising one or more (e.g., one, two or three) VH CDRs and one or more (e.g., one, two or three) VL CDRs listed in Tables 1-3. In particular, described herein is CD47 binding agent (e.g., antibody, such as a bispecific antibody) comprising: a VH CDR1 (SEQ ID NOS: 1, 7, 12, 13, 18, 27, 33, 38, 39, 44, 53, 59, 64, 65, or 70) and a VL CDR1 (SEQ ID NOS: 4, 10, 16, 21, 30, 36, 42, 47, 56, 62, 68, or 73); a VH CDR1 (SEQ ID NOS: 1, 7, 12, 13, 18, 27, 33, 38, 39, 44, 53, 59, 64, 65, or 70) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76) and a VL CDR1 (SEQ ID NOS: 4, 10, 16, 21, 30, 36, 42, 47, 56, 62, 68, or 73); a VH CDR2 (SEQ ID NOS: 2, 8, 14, 19, 24, 28, 34, 40, 45, 50, 54, 60, 66, 71, or 76) and a VL CDR2 (SEQ ID NOS: 5, 11, 22, 31, 37, 48, 57, 63, or 74); a VH CDR2 (SEQ ID NOS: 2, 8, 14, 19, 24, 28, 34, 40, 45, 50, 54, 60, 66, 71, or 76) and a VL CDR3 (SEQ ID NOS: 6, 17, 23, 32, 43, 49, 58, 69, or 75); a VH CDR3 (SEQ ID NOS: 3, 9, 15, 20, 29, 35, 41, 46, 55, 61, 67, or 72) and a VL CDR1 (SEQ ID NOS: 4, 10, 16, 21, 30, 36, 42, 47, 56, 62, 68, or 73); a VH CDR3 (SEQ ID NOS: 3, 9, 15, 20, 29, 35, 41, 46, 55, 61, 67, or 72) and a VL CDR2 (SEQ ID NOS: 5, 11, 22, 31, 37, 48, 57, 63, or 74); a VH CDR3 (SEQ ID NOS: 3, 9, 15, 20, 29, 35, 41, 46, 55, 61, 67, or 72) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76) and a VL CDR1 (SEQ ID NOS: 4, 10, 16, 21, 30, 36, 42, 47, 56, 62, 68, or 73); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR1 (SEQ ID NOS: 4, 10, 16, 21, 30, 36, 42, 47, 56, 62, 68, or 73), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR2 (SEQ ID NOS: 5, 11, 22, 31, 37, 48, 57, 63, or 74); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR2 (SEQ ID NOS: 2, 8, 14, 19, 24, 28, 34, 40, 45, 50, 54, 60, 66, 71, or 76), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR3 (SEQ ID NOS: 3, 9, 15, 20, 29, 35, 41, 46, 55, 61, 67, or 72), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR1 (SEQ ID NOS: 4, 10, 16, 21, 30, 36, 42, 47, 56, 62, 68, or 73); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR3 (SEQ ID NOS: 3, 9, 15, 20, 29, 35, 41, 46, 55, 61, 67, or 72), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR2 (SEQ ID NOS: 5, 11, 22, 31, 37, 48, 57, 63, or 74); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR2 (SEQ ID NOS: 5, 11, 22, 31, 37, 48, 57, 63, or 74) and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR2 (SEQ ID NOS: 2, 8, 14, 19, 24, 28, 34, 40, 45, 50, 54, 60, 66, 71, or 76), a VL CDR1 (SEQ ID NOS: 4, 10, 16, 21, 30, 36, 42, 47, 56, 62, 68, or 73), a VL CDR2 (SEQ ID NOS: 5, 11, 22, 31, 37, 48, 57, 63, or 74), and a VL CDR3 (SEQ ID NOS: 6, 17, 23, 32, 43, 49, 58, 69, or 75); a VH CDR1 (SEQ ID NOS: 1, 7, 12, 13, 18, 27, 33, 38, 39, 44, 53, 59, 64, 65, or 70), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73), a VL CDR2 (SEQ ID NOS: 5, 11, 22, 31, 37, 48, 57, 63, or 74), and a VL CDR3 (SEQ ID NOS: 6, 17, 23, 32, 43, 49, 58, 69, or 75); a VH CDR2 (SEQ ID NOS: 2, 8, 14, 19, 24, 28, 34, 40, 45, 50, 54, 60, 66, 71, or 76), a VH CDR3 (SEQ ID NOS: 3, 9, 15, 20, 29, 35, 41, 46, 55, 61, 67, or 72), a VL CDR1 (SEQ ID NOS: 4, 10, 16, 21, 30, 36, 42, 47, 56, 62, 68, or 73), a VL CDR2 (SEQ ID NOS: 5, 11, 22, 31, 37, 48, 57, 63, or 74), and a VL CDR3 (SEQ ID NOS: 6, 17, 23, 32, 43, 49, 58, 69, or 75); or any combination thereof of the VH CDRs (SEQ ID NOS: 1, 7, 12, 13, 18, 27, 33, 38, 39, 44, 53, 59, 64, 65, 70, 2, 8, 14, 19, 24, 28, 34, 40, 45, 50, 54, 60, 66, 71, 76, 3, 9, 15, 20, 29, 35, 41, 46, 55, 61, 67, or 72) and VL CDRs (SEQ ID NOS: 4, 10, 16, 21, 30, 36, 42, 47, 56, 62, 68, 73, 5, 11, 22, 31, 37, 48, 57, 63, 74, 6, 17, 23, 32, 43, 49, 58, 69, or 75) listed in Tables 1-3.

In some embodiments, described herein is an antibody or fragment thereof that binds to CD47, wherein the antibody or fragment thereof comprises: (a) a heavy chain variable (VH) region comprising: (1) a VH CDR1 having an amino acid sequence selected from the group consisting of: (i) SEQ ID NO:1, 27, or 53, (ii) SEQ ID NO:7, 33, or 59, (iii) SEQ ID NO:12, 38, or 64, (iv) SEQ ID NO:13, 39, or 65, and (v) SEQ ID NO:18, 44, or 70; (2) a VH CDR2 having an amino acid sequence selected from the group consisting of: (i) SEQ ID NO:2, 28, or 54, (ii) SEQ ID NO:8, 34, or 60, (iii) SEQ ID NO:14, 40, or 66, (iv) SEQ ID NO:19, 45, or 71, and (v) SEQ ID NO:24, 50, or 76; and (3) a VH CDR3 having an amino acid sequence selected from the group consisting of: (i) SEQ ID NO:3, 29, or 55, (ii) SEQ ID NO:9, 35, or 61, (iii) SEQ ID NO:15, 41, or 67, (iv) SEQ ID NO:20, 46, or 72; and/or (b) a light chain variable (VL) region comprising: (1) a VL CDR1 having an amino acid sequence selected from the group consisting of: (i) SEQ ID NO:4, 30, or 56, (ii) SEQ ID NO:10, 36, or 62, (iii) SEQ ID NO:16, 42, or 68, an (iv) SEQ ID NO:21, 47, or 73; (2) a VL CDR2 having an amino acid sequence selected from the group consisting of: (i) SEQ ID NO:5, 31 or 57, (ii) SEQ ID NO:11, 37, or 63, an (iii) SEQ ID NO:22, 48, or 74; and (3) a VL CDR3 having an amino acid sequence selected from the group consisting of: (i) SEQ ID NO:6, 32 or 58, (ii) SEQ ID NO:17, 43, or 69, an (iii) SEQ ID NO:23, 49, or 75.

In some embodiments, described herein is an antibody or fragment thereof that binds to CD47, wherein the antibody or fragment thereof comprises a heavy chain variable (VH) region comprising: (1) a VH CDR1 having an amino acid sequence selected from the group consisting of: (i) SEQ ID NO:1, 27, or 53, (ii) SEQ ID NO:7, 33, or 59, (iii) SEQ ID NO:12, 38, or 64, (iv) SEQ ID NO:13, 39, or 65, and (v) SEQ ID NO:18, 44, or 70; (2) a VH CDR2 having an amino acid sequence selected from the group consisting of: (i) SEQ ID NO:2, 28, or 54, (ii) SEQ ID NO:8, 34, or 60, (iii) SEQ ID NO:14, 40, or 66, (iv) SEQ ID NO:19, 45, or 71, and (v) SEQ ID NO:24, 50, or 76; and (3) a VH CDR3 having an amino acid sequence selected from the group consisting of: (i) SEQ ID NO:3, 29, or 55, (ii) SEQ ID NO:9, 35, or 61, (iii) SEQ ID NO:15, 41, or 67, and (iv) SEQ ID NO:20, 46, or 72.

In some embodiments, described herein is an antibody or fragment thereof that binds to CD47, wherein the antibody or fragment thereof comprises a light chain variable (VL) region comprising: (1) a VL CDR1 having an amino acid sequence selected from the group consisting of: (i) SEQ ID NO:4, 30, or 56, (ii) SEQ ID NO:10, 36, or 62, (iii) SEQ ID NO:16, 42, or 68, and (iv) SEQ ID NO:21, 47, or 73; (2) a VL CDR2 having an amino acid sequence selected from the group consisting of: (i) SEQ ID NO:5, 31, or 57, (ii) SEQ ID NO:11, 37, or 63, and (iii) SEQ ID NO:22, 48, or 74; and (3) a VL CDR3 having an amino acid sequence selected from the group consisting of: (i) SEQ ID NO:6, 32, or 58, (ii) SEQ ID NO:17, 43, or 69, and (iii) SEQ ID NO:23, 49, or 75.

In some embodiments, described herein is an antibody or fragment thereof that binds to CD47 comprising all three heavy chain complementarity determining regions (CDRs) and/or all three light chain CDRs from: the antibody designated C40 that comprises a VH sequence that is SEQ ID NO:25 and a VL sequence that is SEQ ID NO:26; the antibody designated C56 that comprises a VH sequence that is SEQ ID NO:51 and a VL sequence that is SEQ ID NO:52; or the antibody designated C59 that comprises a VH sequence that is SEQ ID NO:77 and a VL sequence that is SEQ ID NO:78. In some embodiments, the antibody or fragment thereof comprises all three heavy chain CDRs and/or all three light chain CDRs from the antibody designated C40. In some embodiments, antibody or fragment thereof comprises all three heavy chain CDRs and/or all three light chain CDRs from the antibody designated C56. In some embodiments, the antibody or fragment thereof comprises all three heavy chain CDRs and/or all three light chain CDRs from the antibody designated C59.

In some embodiments, described herein is an antibody or fragment thereof that binds to CD47, wherein the antibody comprises: (a) a heavy chain variable (VH) region comprising a VH CDR1, a VH CDR2, and a VH CDR3 amino acid sequence depicted in Tables 1-3; and/or (b) a light chain variable (VL) region comprising a VL CDR1, a VL CDR2, and a VL CDR3 amino acid sequence depicted in Tables 1-3. In some embodiments, the antibody comprises a heavy chain variable (VH) region comprising a VH CDR1, a VH CDR2, and a VH CDR3 amino acid sequence depicted in Tables 1-3. In some embodiments, the antibody comprises a light chain variable (VL) region comprising a VL CDR1, a VL CDR2, and a VL CDR3 amino acid sequence depicted in Tables 1-3.

In some embodiments, described herein is an antibody comprising: (a) a heavy chain variable (VH) region comprising: (1) a VH CDR1 having an amino acid sequence selected from the group consisting of SEQ ID NO:1, 7, 12, 13, and 18; (2) a VH CDR2 having an amino acid sequence selected from the group consisting of SEQ ID NO:2, 8, 14, 19, and 24; and (3) a VH CDR3 having an amino acid sequence selected from the group consisting of SEQ ID NO:3, 9, 15, and 20; and (b) a light chain variable (VL) region comprising: (1) a VL CDR1 having an amino acid sequence selected from the group consisting of SEQ ID NO:4, 10, 16, and 21; (2) a VL CDR2 having an amino acid sequence selected from the group consisting of SEQ ID NO:5, 11, and 22; and (3) a VL CDR3 having an amino acid sequence selected from the group consisting of SEQ ID NO:6, 17, and 23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a light chain variable (VL) region comprising: (1) a VL CDR1 having the amino acid sequence of SEQ ID NO:10; (2) a VL CDR2 having the amino acid sequence of SEQ ID NO:11; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:12; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:13; (2) a VH CDR2 having the amino acid sequence of SEQ ID NO:14; and (3) a VH CDR3 having the amino acid sequence of SEQ ID NO:15; and (b) a light chain variable (VL) region comprising: (1) a VL CDR1 having the amino acid sequence of SEQ ID NO:16; (2) a VL CDR2 having the amino acid sequence of SEQ ID NO:11; and (3) a VL CDR3 having the amino acid sequence of SEQ ID NO:17.

In some embodiments, described herein is an antibody comprising: (a) a heavy chain variable (VH) region comprising: (1) a VH CDR1 having the amino acid sequence of SEQ ID NO:18; (2) a VH CDR2 having the amino acid sequence of SEQ ID NO:19; and (3) a VH CDR3 having the amino acid sequence of SEQ ID NO:20; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:24; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having an amino acid sequence selected from the group consisting of SEQ ID NO:27, 33, 38, 39, and 44; (2) a VH CDR2 having an amino acid sequence selected from the group consisting of SEQ ID NO:28, 34, 40, 45, and 50; and (3) a VH CDR3 having an amino acid sequence selected from the group consisting of SEQ ID NO:29, 35, 41, and 46; and (b) a light chain variable (VL) region comprising: (1) a VL CDR1 having an amino acid sequence selected from the group consisting of SEQ ID NO:30, 36, 42, and 47; (2) a VL CDR2 having an amino acid sequence selected from the group consisting of SEQ ID NO:31, 37, and 48 and (3) a VL CDR3 having an amino acid sequence selected from the group consisting of SEQ ID NO:32, 43, and 49.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32.

In some embodiments, described herein is an antibody comprising: (a) a heavy chain variable (VH) region comprising: (1) a VH CDR1 having the amino acid sequence of SEQ ID NO:33; (2) a VH CDR2 having the amino acid sequence of SEQ ID NO:34; and (3) a VH CDR3 having the amino acid sequence of SEQ ID NO:35; and (b) a light chain variable (VL) region comprising: (1) a VL CDR1 having the amino acid sequence of SEQ ID NO:36; (2) a VL CDR2 having the amino acid sequence of SEQ ID NO:37; and (3) a VL CDR3 having the amino acid sequence of SEQ ID NO:32.

In some embodiments, described herein is an antibody comprising: (a) a heavy chain variable (VH) region comprising: (1) a VH CDR1 having the amino acid sequence of SEQ ID NO:38; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32.

In some embodiments, described herein is an antibody comprising: (a) a heavy chain variable (VH) region comprising: (1) a VH CDR1 having the amino acid sequence of SEQ ID NO:39; (2) a VH CDR2 having the amino acid sequence of SEQ ID NO:40; and (3) a VH CDR3 having the amino acid sequence of SEQ ID NO:41; and (b) a light chain variable (VL) region comprising: (1) a VL CDR1 having the amino acid sequence of SEQ ID NO:42; (2) a VL CDR2 having the amino acid sequence of SEQ ID NO:37; and (3) a VL CDR3 having the amino acid sequence of SEQ ID NO:43.

In some embodiments, described herein is an antibody comprising: (a) a heavy chain variable (VH) region comprising: (1) a VH CDR1 having the amino acid sequence of SEQ ID NO:44; (2) a VH CDR2 having the amino acid sequence of SEQ ID NO:45; and (3) a VH CDR3 having the amino acid sequence of SEQ ID NO:46; and (b) a light chain variable (VL) region comprising: (1) a VL CDR1 having the amino acid sequence of SEQ ID NO:47; (2) a VL CDR2 having the amino acid sequence of SEQ ID NO:48; and (3) a VL CDR3 having the amino acid sequence of SEQ ID NO:49.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:50; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:31; and (3) a VL CDR3 having the amino acid sequence of SEQ ID NO:32. In some embodiments, described herein is an antibody comprising: (a) a heavy chain variable (VH) region comprising: (1) a VH CDR1 having an amino acid sequence selected from the group consisting of SEQ ID NO:53, 59, 64, 65, and 70; (2) a VH CDR2 having an amino acid sequence selected from the group consisting of SEQ ID NO:54, 60, 66, 71, and 76; and (3) a VH CDR3 having an amino acid sequence selected from the group consisting of SEQ ID NO:55, 61, 67, and 72; and (b) a light chain variable (VL) region comprising: (1) a VL CDR1 having an amino acid sequence selected from the group consisting of SEQ ID NO:56, 62, 68, and 73; (2) a VL CDR2 having an amino acid sequence selected from the group consisting of SEQ ID NO:57, 63 and 74; and (3) a VL CDR3 having an amino acid sequence selected from the group consisting of SEQ ID NO:58, 69, and 75.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:54; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:59; (2) a VH CDR2 having the amino acid sequence of SEQ ID NO:60; and (3) a VH CDR3 having the amino acid sequence of SEQ ID NO:61; and (b) a light chain variable (VL) region comprising: (1) a VL CDR1 having the amino acid sequence of SEQ ID NO:62; (2) a VL CDR2 having the amino acid sequence of SEQ ID NO:63; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:64; (2) a VH CDR2 having the amino acid sequence of SEQ ID NO:54; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:65; (2) a VH CDR2 having the amino acid sequence of SEQ ID NO:66; and (3) a VH CDR3 having the amino acid sequence of SEQ ID NO:67; and (b) a light chain variable (VL) region comprising: (1) a VL CDR1 having the amino acid sequence of SEQ ID NO:68; (2) a VL CDR2 having the amino acid sequence of SEQ ID NO:63; and (3) a VL CDR3 having the amino acid sequence of SEQ ID NO:69.

In some embodiments, described herein is an antibody comprising: (a) a heavy chain variable (VH) region comprising: (1) a VH CDR1 having the amino acid sequence of SEQ ID NO:70; (2) a VH CDR2 having the amino acid sequence of SEQ ID NO:71; and (3) a VH CDR3 having the amino acid sequence of SEQ ID NO:72; and (b) a light chain variable (VL) region comprising: (1) a VL CDR1 having the amino acid sequence of SEQ ID NO:73; (2) a VL CDR2 having the amino acid sequence of SEQ ID NO:74; and (3) a VL CDR3 having the amino acid sequence of SEQ ID NO:75.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:76; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising a VH region and/or VL region described herein, which further comprises human framework sequences. In some embodiment, the VH region and/or VL region further comprises a framework 1 (FR1), a framework 2 (FR2), a framework 3 (FR3) and/or a framework 4 (FR4) sequence.

In some embodiments, the antibody described herein is a monoclonal antibody. In some embodiments, the monoclonal antibody is a humanized, human or chimeric antibody. In some embodiments, the antibody described herein is a Fab, Fab′, F(ab′)2, Fv, scFv, (scFv)2, single chain antibody molecule, dual variable region antibody, single variable region antibody, linear antibody, V region, or a multispecific antibody formed from antibody fragments.

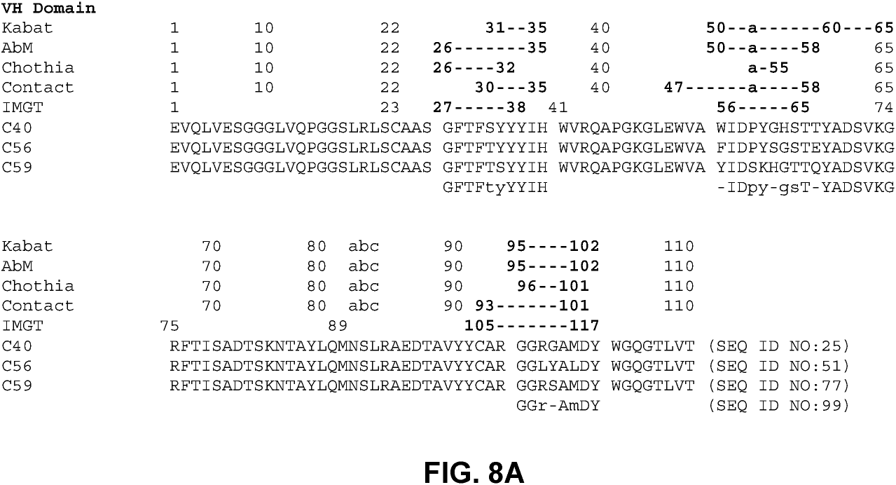

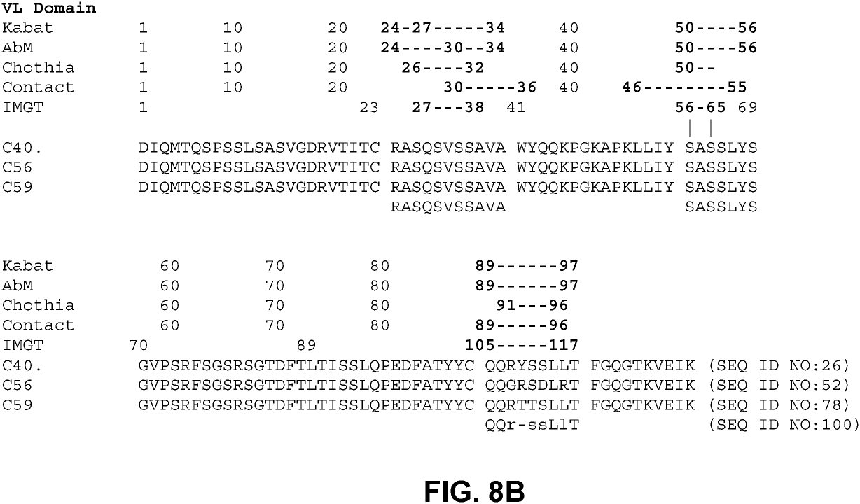

In some embodiments, the CDRs disclosed herein include consensus sequences derived from groups of related antibodies (see, e.g., Tables 1-3). As described herein, a “consensus sequence” refers to amino acid sequences having conserved amino acids common among a number of sequences and variable amino acids that vary within a given amino acid sequences. The CDR consensus sequences provided include CDRs corresponding to CDRH1, CDRH2, CDRH3, CDRL1, CDRL2 and/or CDRL3. Consensus sequences of CDRs of CD47 binding agents (e.g., antibodies, such as bispecific antibodies) are shown in FIGS. 8A and 8B. Accordingly, in some embodiments, a CD47 binding agent (e.g., an antibody, such as a bispecific antibody) described herein comprises (a) a heavy chain variable (VH) region comprising: (1) a VH CDR1 having the amino acid sequence GFTFX₁X₂YYIH (SEQ ID NO:83), wherein X₁ and X₂ are each independently a naturally occurring amino acid; (2) a VH CDR2 having the amino acid sequence of X₁IDX₂X₃X₄X₅XeTX₇YADSVKG (SEQ ID NO:84), wherein X₁, X₂, X₃ X₄, X₅, X₆, and X₇ are each independently a naturally occurring amino acid; and (3) a VH CDR3 having the amino acid of GGX₁X₂AX₃DY (SEQ ID NO:85), wherein X₁, X₂, and X₃ are each independently a naturally occurring amino acid, and/or (b) a light chain variable (VL) region comprising: (1) a VL CDR1 having the amino acid sequence RASQSVSSAVA (SEQ ID NO:86); (2) a VL CDR2 having the amino acid sequence SASSLYS (SEQ ID NO:87); and (3) a VL CDR3 having the amino acid sequence QQX₁X₂X₃X₄LX₅T (SEQ ID NO:88), wherein X₁, X₂, X₃ X₄, and X₅ are each independently a naturally occurring amino acid. In some embodiments, the VH CDR1 of a CD47 binding agent described herein has the amino acid sequence of GFTFX₁X₂YYIH (SEQ ID NO:101), wherein X₁ is a S or T, and X₂ is a Y or S. In some embodiments, the VH CDR2 of a CD47 binding agent described herein has the amino acid sequence of X₁IDX₂X₃X₄X₅X₆TX₇YADSVKG (SEQ ID NO:89), wherein X₁ is a W, F or Y, X₂ is a P or S, X₃ is a Y or K, X₄ is a G, S or H, X₅ is a H or G, X₆ is a S or T, and X₇ is a T, E or Q. In some embodiments, the VH CDR3 of a CD47 binding agent described herein has the amino acid sequence of GGX₁X₂AX₃DY (SEQ ID NO:90), wherein X₁ is a R or L, X₂ is a G, Y or S, and X₃ is a M or L. In some embodiments, the VL CDR1 of a CD47 binding agent described herein has the amino acid sequence of RASQSVSSAVA (SEQ ID NO:86). In some embodiments, the VL CDR2 of a CD47 binding agent described herein has the amino acid sequence of SASSLYS (SEQ ID NO:87). In some embodiments, the VL CDR3 of a CD47 binding agent described herein has the amino acid sequence of QQX₁X₂X₃X₄LX₅T (SEQ ID NO:91), wherein X₁ is a R or G, X₂ is Y, R or T, X₃ is a S or T, X₄ is a S or D, and Xs is a L or R.

In some embodiments, described herein is a binding agent that binds to essentially the same epitope as an antibody or fragment thereof of any one of the antibodies described herein. In some embodiments, described hereins is a binding agent that competes for binding to human CD47 with an antibody or fragment thereof of any one described herein. In some embodiments, the binding agent is an antibody or fragment thereof.

In certain aspects, the CDRs of a CD47 binding agent (e.g., an antibody), including a human CD47 binding agent, can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain aspects, the CDRs of a CD47 binding agent (e.g., an antibody), including a human CD47 binding agent, can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226).

In certain aspects, the CDRs of a CD47 binding agent (e.g., an antibody), including a human CD47 binding agent, can be determined according to the ImMunoGeneTics (IMGT) system, for example, as described in Lefranc, M.-P., 1999, The Immunologist, 7:132-136 and Lefranc, M.-P. et al., 1999, Nucleic Acids Res., 27:209-212 (“IMGT CDRs”).

In certain aspects, the CDRs of a CD47 binding agent (e.g., an antibody), including a human CD47 binding agent, can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of a CD47 binding agent (e.g., an antibody), including a human CD47 binding agent, can be determined according to the Contact system, which will be referred to herein as the “Contact CDRs” (see, e.g., MacCallum RM et al., 1996, J Mol Biol 5: 732-745). The Contact CDRs are based on an analysis of the available complex crystal structures.

In some embodiments, the position of one or more CDRs along the VH (e.g., CDR1, CDR2, or CDR3) and/or VL (e.g., CDR1, CDR2, or CDR3) region of a CD47 binding agent (e.g., an antibody), including a human CD47 binding agent, described herein may vary by one, two, three, four, five, or six amino acid positions so long as binding to CD47 (e.g., human CD47) is maintained (e.g., substantially maintained, for example, at least 50%, at least 60%, at least 70%, at least 80%, at least 90%, at least 95%). For example, in some embodiments, the position defining a CDR of any of Table 1 or 2 may vary by shifting the N-terminal and/or C-terminal boundary of the CDR by one, two, three, four, five, or six amino acids, relative to the current CDR position, so long as binding to CD47 (e.g., human CD47) is maintained (e.g., substantially maintained, for example, at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of a CD47 binding agent (e.g., an antibody), including a human CD47 binding agent, described herein may vary (e.g., be shorter or longer) by one, two, three, four, five, or more amino acids, so long as binding to CD47 (e.g., human CD47) is maintained (e.g., substantially maintained, for example, at least 50%, at least 60%, at least 70%, at least 80%, at least 90%, at least 95%). For example, in some embodiments, a VH and/or VL CDR1, CDR2, and/or CDR3 described herein may be one, two, three, four, five or more amino acids shorter than one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). In other embodiments, a VH and/or VL CDR1, CDR2, and/or CDR3 described herein may be one, two, three, four, five or more amino acids longer than one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be extended by one, two, three, four, five or more amino acids compared to one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). In other embodiments, the carboxy terminus of a VH and/or VL CDR1, CDR2, and/or CDR3 described herein may be extended by one, two, three, four, five or more amino acids compared to one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be shortened by one, two, three, four, five or more amino acids compared to one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). In some embodiments, the carboxy terminus of a VH and/or VL CDR1, CDR2, and/or CDR3 described herein may be shortened by one, two, three, four, five or more amino acids compared to one or more of the CDRs described by SEQ ID NOS: 1-24, 27-50, or 53-76, so long as binding to CD47 (e.g., human CD47) is maintained (e.g., substantially maintained, for example, at least 50%, at least 60%, at least 70%, at least 80%, at least 90%, at least 95%). Any method known in the art can be used to ascertain whether binding to CD47 (e.g., human CD47) is maintained, for example, the binding assays and conditions described in the “Examples” section described herein. For example, Example 2 described herein describes an assay for measuring binding to CD47 (e.g., human CD47).

In other embodiments, the CD47 binding agents (e.g., antibodies), including human CD47 binding agents, presented herein that bind to CD47, comprise conservative sequence modifications. With respect to polypeptides that are CD47 binding agents (e.g., antibodies), such as human CD47 binding agents, conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, a predicted nonessential amino acid residue in a CD47 is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of the CD47 binding agents (e.g., antibodies), including human CD47 binding agents, by 50%, or 55%, or 60%, or 65%, or 70%, or 75%, or 80%, or 85%, or 90%, or 95%, or 98%, or 99%. In some embodiments, the nucleotide and amino acid sequence modifications refer to at most 1, 2, 3, 4, 5, or 6 amino acid substitutions to the CDRs described in Table 1, Table 2, or Table 3. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution.

The present disclosure provides CD47 binding agents (e.g., antibodies) with a masking moiety and/or cleavable moiety in which one or more of the CD47 binding domains of the CD47 binding agent (e.g., antibody) are masked (e.g., via a masking moiety) and/or activatable (e.g., via a cleavable moiety). Technologies for masking of a CD47 binding agent (e.g., an antibody) are well known in the art, including SAFE body masking technology (see, e.g., U.S. Pat. Application Publication No. 2019/0241886) and Probody masking technology (see, e.g., U.S. Pat. Application Publication No. 2015/0079088). Such technologies can be used to generate a CD47 binding agent (e.g., an antibody) that is masked and/or activatable. Such masked and/or activatable CD47 binding agents (e.g., antibodies) are useful for the preparation of conjugates, including immunoconjugates, antibody-drug conjugates (ADCs), masked ADCs and activatable antibody-drug conjugates (AADCs), comprising any one of the CD47 binding agents (e.g., antibodies), such as human CD47 binding agents, of the present disclosure, including those directly or indirectly linked another agent such as a drug. For example, CD47 binding agents (e.g., antibodies), such as human CD47 binding agents, of the present disclosure may be covalently bound by a synthetic linker to one or more agents such as drugs.

If desired, a CD47 binding agent (e.g., an antibody), including a human CD47 binding agent, is linked or conjugated (directly or indirectly) to a moiety with effector function, such as cytotoxic activity (e.g., a chemotherapeutic moiety or a radioisotope) or immune recruitment activity. Moieties that are linked or conjugated (directly or indirectly) include drugs that are cytotoxic (e.g., toxins such as aurostatins) or non-cytotoxic (e.g., signal transduction modulators such as kinases or masking moieties that mask one or more binding domains of a CD47 binding agent (e.g., antibody), or cleavable moieties that allow for activating a CD47 binding agent by cleaving of a cleavable moiety to unmask one or more binding domains of a CD47 binding agent (e.g., antibody) in the tumor microenvironment) in the form of masked conjugates. Moieties that promote immune recruitment can include other antigen-binding agents, such as viral proteins that bind selectively to cells of the innate immune system. Alternatively or in addition, a CD47 binding agent (e.g., an antibody), including a human CD47 binding agent, is optionally linked or conjugated (directly or indirectly) to a moiety that facilitates isolation from a mixture (e.g., a tag) or a moiety with reporter activity (e.g., a detection label or reporter protein). It will be appreciated that the features of a CD47 binding agent (e.g., an antibody), including a human CD47 binding agent, described herein extend also to a polypeptide comprising a CD47 binding agent fragment.

In some embodiments, CD47 binding agents (e.g., antibodies), including CD47 binding agents, described herein which bind to human CD47, may be linked or conjugated (directly or indirectly) to a polypeptide, which can result in the generation of an activatable antibody. In some embodiments, a CD47 binding agent (e.g., an antibody), including a human CD47 binding agent, is linked or conjugated (directly or indirectly) to an agent. In some embodiments, the agent is a drug, resulting in an ADC or an AADC when the antibody of the ADC comprises a masking moiety and a cleavable moiety.

In some embodiments, CD47 binding agents (e.g., antibodies), including human CD47 binding agents, described herein are conjugated or recombinantly linked (directly or indirectly) to a therapeutic agent (e.g., a cytotoxic agent) or to a diagnostic or detectable agent. The conjugated or recombinantly linked antibodies, including masked or activatable conjugates, can be useful, for example, for treating or preventing a disease or disorder such as a phagocytic cell dysfunctional disease, disorder or condition. The conjugated or recombinantly linked CD47 binding agents (e.g., antibodies), including masked or activatable conjugates, can be useful, for example, for monitoring or prognosing the onset, development, progression, and/or severity of a phagocytic cell dysfunctional disease, including a CD47-expressing tumor cell.

Such diagnosis and detection can be accomplished, for example, by coupling a CD47 binding agent (e.g., an antibody) to detectable substances including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (1311, 125I, 1231, and 1211), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Also described herein are CD47 binding agents (e.g., antibodies) that are recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In particular, described herein are fusion proteins comprising an antigen-binding fragment of a CD47 binding agent (e.g., an antibody), including a human CD47 binding agent, described herein (e.g., comprising CDR1, CDR2, and/or CDR3 of VH and/or VL) and a heterologous protein, polypeptide, or peptide. In some embodiments, the heterologous protein, polypeptide, or peptide that a CD47 binding agent (e.g., an antibody) is linked to is useful for targeting the CD47 binding agent to a particular cell (e.g., a CD47-expressing cell, including a tumor cell).

Moreover, CD47 binding agents (e.g., antibodies), including human CD47 binding agents, described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, a CD47 binding agent (e.g., an antibody) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody (e.g., a non-CD47 binding antibody)). In certain embodiments, the fusion protein retains the biological activity of a CD47 binding agent. In certain embodiments, the fusion protein comprises a CD47 antibody VH region, VL region, VH CDR (one, two or three VH CDRs), and/or VL CDR (one, two or three VL CDRs), wherein the fusion protein binds to a CD47 epitope, a CD47 fragment and/or a CD47 polypeptide.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of CD47 binding agents (e.g., antibodies), including human CD47 binding agents, as described herein, including, for example, CD47 binding agents with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). In some embodiments, CD47 binding agents, including human CD47 binding agents, may be altered by being subjected to random mutagenesis by error-prone PCR, random nucleotide insertion, or other methods prior to recombination. A polynucleotide encoding a CD47 binding agent described herein may be recombined with one or more components, motifs, sections, parts, domains, fragments, etc. of one or more heterologous molecules.

CD47 binding agents (e.g., antibodies), including human CD47 binding agents, described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

CD47 binding agents (e.g., antibodies), including human CD47 binding agents, described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., antibody-drug conjugates) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of ADC or an AADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents, including wherein the agent is a drug for the preparation of ADC or AADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC or AADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

In some embodiments, a CD47 binding agent (e.g., an antibody), including a human CD47 binding agent, described herein is conjugated to a cytotoxic agent. In some embodiments, a CD47 binding agent (e.g., an antibody), including a human CD47 binding agent, disclosed herein can be optionally conjugated with one or more cytotoxic agent(s) disclosed herein or known in the art in order to generate an ADC or AADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, 90Y, 125I, 1311, 123I, 111In, 131In, 105Rh, 153Sm, 67Cu, 67Ga, 166Ho, 177Lu, 186Re, 188Re, and 212Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, a CD47 binding agent (e.g., an antibody), including a human CD47 binding agent, described herein is conjugated to a drug such as a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In some embodiments, the mitotic inhibitor is a dolastatin, an auristatin, a maytansinoid, and a plant alkaloid. In some embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. In some embodiments, the actinomycine is a pyrrolobenzodiazepine (PBD).

CD47 binding agents (e.g., antibodies), including human CD47 binding agents, described herein may be monospecific, bispecific, trispecific or of greater multispecificity. Such agents may include antibodies. Multispecific antibodies, such as bispecific antibodies, are monoclonal antibodies that have binding specificities for at least two different targets (e.g., antigens) or two different epitopes on the same target (e.g., a bispecific antibody directed to CD47 with a first binding domain for a first epitope of a CD47, and a second binding domain for a second epitope of CD47). In some embodiments, the multispecific (e.g., bispecific) antibodies can be constructed based on the sequences of the antibodies described herein, e.g., the CDR sequences listed in Table 1, Table 2, and Table 3. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for CD47 and the other is for any other target (e.g., antigen). In some embodiments, a multispecific (e.g., bispecific) antibody can comprise more than one target (e.g., antigen) binding domain, in which different binding domains are specific for different targets (e.g., a first binding domain that binds to CD47 and a second binding domain that binds another target (e.g., antigen), such as an immune checkpoint regulator (e.g., a negative checkpoint regulator). In some embodiments, multispecific (e.g., bispecific) antibody molecules can bind than one (e.g., two or more) epitopes on the same target (e.g., antigen). In some embodiments, one of the binding specificities is CD47 and the other is for one or more of Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, and CGEN-15092.

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10: 1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C- terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four- in-one). See Spiess et al. Mol. Immunol. 67(2015):95-106. In some embodiments, an exemplary antibody format is a B-Body format for monospecific or multispecific (e.g., bispecific antibodies) as described in e.g. International Patent Application Publication No. WO 2018/075692 and U.S. Pat. Application Publication No. 2018/0118811.

Bispecific antibody fragments (BsAb) are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody- HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be extend the serum half-life of antibody fragments. In embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, CD47 binding agents (e.g., antibodies), including human CD47 binding agents, disclosed herein can be provided in any antibody format disclosed herein or known in the art. As a non-limiting example, in some embodiments, the CD47 binding agents (e.g., antibodies), including human CD47 binding agents, can be selected from Fabs-in-tandem-Ig (FIT-Ig); DVD-Ig; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody = Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-Ig = dual variable domain immunoglobulin (Abbott); kappa-lambda bodies; TBTI = tetravalent bispecific tandem Ig; and CrossMab (Roche).

In some embodiments, a multispecific (e.g., bispecific) antibody disclosed herein comprises a CD47 binding domain and one or more additional binding domains that bind to one or more targets that are not CD47. In some embodiments, a multispecific (e.g., bispecific) antibody disclosed herein comprises a CD47 binding domain thate.g. comprises the VH and/or VL amino acid sequences of Table 1. In some embodiments, a multispecific (e.g., bispecific) antibody disclosed herein comprises a CD47 binding domain that comprises the VH and/or VL amino acid sequences of Table 2. In some embodiments, a multispecific (e.g., bispecific) antibody disclosed herein comprises a CD47 binding domain that comprises the VH and/or VL amino acid sequences of Table 3.

In some embodiments, described herein is a multispecific (e.g., bispecific) antibody comprising a binding domain which binds to CD47 that comprises VH and VL CDRs as set forth in Table 1. In some embodiments, described herein is a multispecific (e.g., bispecific) antibody comprising a binding domain which binds to CD47 that comprises VH and VL CDRs as set for in Table 2. In some embodiments, described herein is a multispecific (e.g., bispecific) antibody comprising a binding domain which binds to CD47 that comprises VH and VL CDRs as set for in Table 3.

Antibodies that bind CD47 may be obtained by any suitable method, such as (but not limited to) immunization with whole tumor cells comprising CD47 and collection of antibodies, recombinant techniques, or screening libraries of antibodies or antibody fragments using CD47 extracellular domain epitopes. Monoclonal antibodies may be generated using a variety of known techniques (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E.coli,” in DNA Cloning 2: Expression Systems, 2nd Edition, Glover et al. (eds.), page 93 (Oxford University Press 1995)). One exemplary technique for generating monoclonal antibodies comprises immunizing an animal with a human CD47 antigen and generating a hybridoma from spleen cells taken from the animal. A hybridoma may produce a monoclonal antibody or antibody fragment that binds CD47.

In a further embodiment, monoclonal antibodies or antibody fragments can be isolated from antibody phage libraries generated using the techniques described in, for example, Antibody Phage Display: Methods and Protocols, P.M. O’Brien and R. Aitken, eds, Humana Press, Totawa N.J., 2002. In principle, synthetic antibody clones are selected by screening phage libraries containing phage that display various fragments of antibody variable region (Fv) fused to phage coat protein. Such phage libraries are screened for against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227: 381-388 (1992).

Screening of the libraries can be accomplished by various techniques known in the art. For example, CD47 (e.g., a CD47 polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

CD47 binding agents (e.g., antibodies) can be obtained by designing a suitable antigen screening procedure to select for the phage clone of interest followed by construction of a full length CD47 binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind CD47 may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455 58, 1997; Jakobovits et al., Ann. N. Y. Acad. Sci., 764: 525 35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies that bind CD47 may be obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. International Patent Publication No. WO 96/30498 discloses the use of the Cre/Lox system to modify the immunoglobulin locus in a mammal, such as to replace all or a portion of the constant or variable region to form a modified antibody molecule. International Patent Publication No. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described in International Patent Publication No. WO 96/33735. The monoclonal antibodies can be tested for the ability to inhibit or neutralize the biological activity or physiological effect of the corresponding protein.

The present disclosure provides humanized antibodies that bind CD47, including human CD47. Humanized antibodies of the present disclosure may comprise one or more CDRs as shown in Tables 1-3. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind CD47 may be produced using techniques known to those skilled in the art (Zhang et al., Molecular Immunology, 42(12): 1445-1451, 2005; Hwang et al., Methods, 36(1): 35-42, 2005; Dall’Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567, all of which are all hereby expressly incorporated herein by reference).

In some cases, the humanized antibodies are constructed by CDR grafting, in which the amino acid sequences of the six complementarity determining regions (CDRs) of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36: 25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623. In some cases, the framework is derived from the consensus sequences of the most abundant human subclasses, V_{L}6 subgroup I (VL61) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169: 1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (Lazar et al., Mol. Immunol. 44: 1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (see, e.g., Foote and Winter, J. Mol. Biol. 224: 487-499, 1992), or from the more limited set of target residues identified by Baca et al. (J. Biol. Chem. 272: 10678-10684, 1997).

In FR shuffling, whole FRs are combined with the non-human CDRs instead of creating combinatorial libraries of selected residue variants (see, e.g., Dall’Acqua et al., Methods 36: 43-60, 2005). The libraries may be screened for binding in a two-step selection process, first humanizing VL, followed by VH. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44: 3049-60, 2007).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple sub-classes with distinct human V-segment CDRs. Humaneering allows for isolation of antibodies that are 91-96 % homologous to human germline gene antibodies. (see, e.g., Alfenito, Cambridge Healthtech Institute’s Third Annual PEGS, The Protein Engineering Summit, 2007).

The “human engineering” method involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody’s folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody’s variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7: 805-814 (1994), U.S. Pats. 5,766,886, 5,770,196, 5,821,123, and 5,869,619, and PCT Application Publication WO 93/11794.

In some embodiments, a CD47 binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate a CD47 binding agent comprising a non-antibody protein scaffold (see, e.g., Simeon and Chen, Protein Cell, 9(1):3-14 (2018); Yang et al., Annu Rev Anal Chem (Palo Alto Calif). 10(1):293-320 (2017)).

Further provided are the materials for generating CD47 binding agents, e.g., human CD47 binding agents, and fragments thereof. For example, an isolated cell (e.g., a hybridoma) may produce a CD47 binding agent (e.g., antibody or antibody fragment). In this regard, a cell (e.g., an isolated cell) may produce an antibody or fragment thereof comprising a VH and a VL as shown in Table 1, 2, or 3 for C40, C56, or C59, respectively. In some embodiments, polynucleotides described herein may comprise one or more nucleic acid sequences encoding a CD47 binding agent (e.g., antibody or antibody fragment). In some embodiments, the polynucleotide is an isolated and/or recombinant polynucleotide. In various aspects, the isolated polynucleotide comprises a nucleotide sequence that encodes an antibody heavy chain variable region (VH) and/or an antibody light chain variable region (V_{L}), wherein the V_{H} and the V_{L} comprise complementarity determining regions (CDRs) identical to CDRs as shown in Table 1, CDRs as shown in Table 2, or CDRs as shown in Table 3.

In some embodiments, one or more vectors (e.g., expression vectors) may comprise one or more polynucleotides for expression of the one or more polynucleotides in a suitable host cell. Such vectors are useful, e.g., for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind CD47 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91: 2687-2692, 1995; and Isner et al., Human Gene Therapy, 7: 989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72(1): 811-816, 1998; Kingsman & Johnson, Scrip Magazine, October, 1998, pp. 43-46); parvoviral vectors, such as adeno-associated viral (AAV) vectors (U.S. Pat. Nos. 5,474,935I; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985, DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Natl. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

An expression vector (or the antibody or fragment thereof discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, 1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243: 375-378, 1989). In some embodiments, the liposome is complexed or employed in conjunction with nuclear nonhistone chromosomal proteins (HMG-1) (Kato et al., J. Biol. Chem., 266: 3361-3364, 1991). In some embodiments, the liposome are complexed or employed in conjunction with both HVJ and HMG-1. Such expression constructs have been successfully employed in transfer and expression of nucleic acid in vitro and in vivo. In some embodiments, a CD47 binding agent (e.g., an antibody), including a human CD47 binding agent, is included in the liposome to target the liposome to cells (such as tumor cells) expressing CD47 on their surface.

A cell may comprise one or more polynucleotides or one or more vectors, e.g., the cell is transformed or transfected with one or more polynucleotides encoding a CD47 binding agent (e.g., an antibody), including a human CD47 binding agent, or the one or more vectors comprising the one or more polynucleotides. In some embodiments, cells express a CD47 binding agent (e.g., an antibody), including a human CD47 binding agent, containing one or more, including six CDRs having at least 75% identity to the CDRs of C40 (see, e.g., Table 1). In some embodiments, the cell expresses a CD47 binding agent (e.g., an antibody), including a human CD47 binding agent, containing the V_{H} and the V_{L} comprising CDRs identical to those of C56 (see, e.g., Table 2). In some embodiments, the cell expresses a CD47 binding agent (e.g., an antibody), including a human CD47 binding agent, containing the V_{H} and the V_{L} comprising CDRs identical to those of C59 (see, e.g., Table 3). The cells may be prokaryotic cells, such as Escherichia coli (see, e.g., Pluckthun et al., Methods Enzymol., 178: 497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., CD47 binding agents (e.g., antibodies), including human CD47 binding agents) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a CD47 binding agent (e.g., an antibody) may comprise culturing a host cell and isolating the CD47 binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327: 70-73, 1987). Several devices for accelerating small particles have been developed. One such device relies on a high voltage discharge to generate an electrical current, which in turn provides the motive force (Yang et al., Proc. Natl. Acad. Sci USA, 87: 9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992); and Current Protocols in Molecular Biology, Ausubel ed., Wiley Interscience, New York, 1999).

A CD47 binding agent (e.g., an antibody), including a human CD47 binding agent, is produced using any suitable method, e.g., isolated from an immunized animal, recombinantly or synthetically generated, or genetically-engineered, including as described above. Antibody fragments derived from an antibody are obtained by, e.g., proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244, 1960; Porter, Biochem. J., 73: 119-127, 1959; U.S. Pat. No. 4,331,647; and by Andrews, S.M. and Titus, J.A. in Current Protocols in Immunology (Coligan et al., eds), John Wiley & Sons, New York (2003), pages 2.8.1 2.8.10 and 2.10A.1 2.10A.5.

A CD47 binding agent (e.g., an antibody), including a human CD47 binding agent, can be genetically engineered. For example, a CD47 binding agent (e.g., an antibody), including a human CD47 binding agent, comprises, for example, a variable region domain generated by recombinant DNA engineering techniques. In this regard, a variable region is optionally modified by insertions, deletions, or changes in the amino acid sequence of the antibody to produce an antibody of interest, including as described above. Polynucleotides encoding complementarity determining regions (CDRs) of interest are prepared, for example, by using polymerase chain reaction to synthesize variable regions using mRNA of antibody producing cells as a template (see, for example, Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2: 106-110, 1991). Current antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

“Humanized antibodies” are antibodies in which CDRs of heavy and light variable chains of non-human immunoglobulins are transferred into a human variable domain. Constant regions need not be present, but if they are, they optionally are substantially identical to human immunoglobulin constant regions, e.g., at least about 85-90%, about 95%, 96%, 97%, 98%, 99% or more identical, in some embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

In some embodiments, CD47 binding agents (e.g., antibodies) described herein are useful in compositions and in methods of treating, preventing, or alleviating a phagocytic cell dysfunctional disease, disorder, or condition, including one or more symptoms of the disease, disorder, or condition. Phagocytic cell dysfunctional diseases, disorders, and conditions tumor immunity and associated cancers, including, but not limited to, any cancer wherein the tumor cells overexpress CD47. Such CD47 over-expressing tumor cells may help tumor cells escape immune surveillance and removal. In addition, CD47 binding agents described herein, such as CD47 binding antibodies (e.g., monospecific or multispecific antibodies, including bispecific antibodies), are useful to inhibit SIRPα signaling and/or enhance phagocytic cell function and thus enhance immune surveillance and removal of tumor cells.

In some embodiments, described herein is a method for treating tumor immunity in a subject comprising administering to the subject a CD47 binding agent (e.g., an antibody) described herein or fragment thereof or a pharmaceutical composition comprising the binding agent (e.g., antibody) described herein.

In some embodiments, described herein is a method for treating a cancer or a tumor in a subject comprising administering to the subject a CD47 binding agent (e.g., an antibody) described herein or fragment thereof or a pharmaceutical composition comprising the binding agent (e.g., antibody) described herein.

In some embodiments, described herein is a method for alleviating one or more symptoms associated with a cancer or a tumor in a subject comprising administering to the subject a CD47 binding agent (e.g., an antibody) described herein or fragment thereof or a pharmaceutical composition comprising the binding agent (e.g., antibody) described herein.

In some embodiments, described herein is a method for decreasing tumor size in a subject with a tumor comprising administering to the subject a CD47 binding agent (e.g., an antibody) described herein or fragment thereof or a pharmaceutical composition comprising the binding agent (e.g., antibody) described herein.

In some embodiments, described herein is a method for treating a phagocytic cell dysfunctional disease, disorder or condition in a subject comprising administering to the subject a CD47 binding agent (e.g., an antibody) described herein or fragment thereof or a pharmaceutical composition comprising the binding agent (e.g., antibody) described herein.

In some embodiments, described herein is a method for increasing immune cell phagocytosis in a subject comprising administering to the subject a CD47 binding agent (e.g., an antibody) described herein or fragment thereof or a pharmaceutical composition comprising the binding agent (e.g., antibody) described herein. In some embodimetns, the immune cell is a macrophage, a neutrophil, a dendritic cell, or a B lymphocyte. In some embodiments, the subject is diagnosed with a cancer or a tumor.

The subject of a method described above can be administered one or more therapeutic agents described herein in combination with a CD47 binding agent (e.g., an antibody) described herein or fragment thereof or a pharmaceutical composition comprising the binding agent (e.g., antibody) described herein.

In some embodiments, the antibody is a human antibody, including, but not limited to, an antibody having variable regions in which both the framework and CDR regions are derived from human germline immunoglobulin sequences as described, for example, in Kabat et al. (1991) Sequences of proteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences, for example, to enhance the activity of the antibody, but do not comprise CDRs derived from other species (e.g., a mouse CDR placed within a human variable framework region).

In some embodiments, a CD47 binding agent (e.g., an antibody) increases phagocytosis and/or enhances phagocytic activity of cells in cell culture. Such cell culture may include tumor cells expressing or overexpressing CD47. Tumor cells include, but are not limited to, breast cancer cells, bladder cancer cells, melanoma cells, prostate cancer cells, mesothelioma cells, lung cancer cells, testicular cancer cells, thyroid cancer cells, squamous cell carcinoma cells, glioblastoma cells, neuroblastoma cells, uterine cancer cells, colorectal cancer cells, and pancreatic cancer cells.

In some embodiments, described herein is a method of enhancing the removal of tumor cells in a subject. For example, the method comprises administering an amount of a CD47 binding agent (e.g., an antibody), such as a human CD47 binding agent described herein, effective to enhance the removal of the tumor cells. In some embodiments, the method includes administering a CD47 binding agent (e.g., an antibody), including a CD47 binding agent, that competes for binding with antibody C40, antibody C56, and/or antibody C59 (see, e.g., CDRs and VH/VL of Tables 1, 2 and/or 3), to human CD47 and/or binds the region of a CD47 recognized by antibody C40, antibody C56, and/or antibody C59 (see, e.g., CDRs and VH/VL of Tables 1, 2 and/or 3), resulting in enhancement of the removal of tumor cells. In some embodiments, one or more binding agents (e.g. antibodies), polynucleotides, vectors, and/or cells as described above can be used in methods of enhancing the removal of tumor cells in vivo (e.g., in a method of treating cancer in a subject).

A method of modulating (e.g., inhibiting, reducing, preventing) tumor growth in a subject also is provided. For example, the method comprises administering to the subject a composition comprising a CD47 binding agent (e.g., an antibody) in an amount effective to modulate tumor growth in the subject. “Tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth. Examples of cancers include, but are not limited to: breast cancer, colon cancer, renal cancer, lung cancer, squamous cell myeloid leukemia, hemangiomas, melanomas, astrocytomas, and glioblastomas as well as other cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinoma, lymphoma, carcinoid tumors, Karposi’s sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm’s tumor (nephroblastoma), lymphoma, leukemia, renal cell carcinoma), bladder and urethra (squamous cell carcinoma, transitional cell carcinoma, adenocarcinoma), prostate (adenocarcinoma, sarcoma, small cell carcinoma of the prostate), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing’s sarcoma, malignant lymphoma (reticulum cell sarcoma), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defornians), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma) serous cystadenocarcinoma, mucinous cystadenocarcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin’s disease, non-Hodgkin’s lymphoma (malignant lymphoma); Skin: malignant melanoma, basal cell carcinoma, squamous cell carcinoma, Karposi’s sarcoma, moles dysplastic nevi, lipoma, angioma, dermatofibroma, keloids, psoriasis; and Adrenal glands: neuroblastoma; as well as cancers of the thyroid including medullary thyroid cancer. Also provided is a method of treating cancer by administering a CD47 binding agent (e.g., an antibody) such as a human CD47 binding agent, to a subject in need thereof, alone or in combination with another agent.

“Enhancing” tumor cell removal does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a CD47 binding agent (e.g., an antibody) of the method.

Additionally, CD47 binding agents (e.g., antibodies) may be used to alleviate or reduce side effects associated with cancer such as, for example, bone deterioration, vertebral collapse, and paralysis. In one aspect, the subject suffers from or is at risk of suffering from bone metastases and a CD47 binding agent (e.g., an antibody) is administered in an amount to reduce deterioration of surrounding bone. Accordingly, in some aspects, a CD47 binding agent prevents bone deterioration due to bone metastases, wherein tumor cell proliferation is or is not reduced. In some aspects, a CD47 binding agent (e.g., an antibody) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

A particular administration regimen of a CD47 binding agent (e.g., an antibody) for a particular subject will depend, in part, upon the agent used, the amount of agent administered, the route of administration, and the cause and extent of any side effects. The amount of agent (e.g., an antibody) administered to a subject (e.g., a mammal, such as a human) should be sufficient to effect the desired response over a reasonable time frame. According, in some embodiments, the amount of a CD47 binding agent (e.g., an antibody) or pharmaceutical composition described herein administered to a subject is an effective amount. In some embodiments, the amount of a CD47 binding agent (e.g., an antibody) or pharmaceutical composition described herein administered to a subject is a therapeutically effective amount. In some aspects, the method comprises administering, e.g., from about 0.1 µg/kg to up to about 100 mg/kg or more. In some embodiments, the dosage ranges from about 1 µg/kg up to about 100 mg/kg; or about 5 µg/kg up to about 100 mg/kg; or about 10 µg/kg up to about 100 mg/kg; or about 1 mg/kg up to about 50 mg/kg; or about 2 mg/kg up to about 30 mg/kg; or about 3 mg/kg up to about 25 mg/kg; or about 3 mg/kg up to about 25 mg/kg; or about 5 mg/kg up to about 10 mg/kg; or about 10 mg/kg up to about 20 mg/kg; or about 10 mg/kg up to about 30 mg/kg. Some conditions or disease states require prolonged treatment, which may or may not entail administering doses of CD47 binding agents (e.g., antibodies), including human CD47 binding agents (e.g., antibodies), over multiple administrations (e.g., every day, three times a week, once a week, once every two weeks, or once every month for a treatment period of three days, seven days, two weeks, three weeks, one month, three months, six months, nine months, 12 months, 15 months, 18 months, 21 months, two years, or more).

Suitable routes of administering a composition comprising a CD47 binding agent (e.g., an antibody), such as a human CD47 binding agent (e.g., an antibody), are well known in the art. Although more than one route can be used to administer an agent (e.g., an antibody), a particular route can provide a more immediate and more effective reaction than another route. Depending on the circumstances, a composition comprising a CD47 binding agent (e.g., an antibody) such as a human CD47 binding agent is applied or instilled into body cavities, absorbed through the skin or mucous membranes, ingested, inhaled, and/or introduced into circulation. For example, it may be desirable to deliver a composition comprising a CD47 binding agent (e.g., an antibody), such as a human CD47 binding agent, through injection by intravenous, subcutaneous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, a CD47 binding agent (e.g., an antibody), such as a human CD47 binding agent, is administered regionally via intraarterial or intravenous administration feeding the region of interest, e.g., via the hepatic artery for delivery to the liver. Alternatively, a CD47 binding agent (e.g., an antibody), such as a human CD47 binding agent, is administered locally via implantation of a membrane, sponge, or another appropriate material on to which the binding agent has been absorbed or encapsulated. Where an implantation device is used, the device is, one aspect, implanted into any suitable tissue or organ, and delivery of a CD47 binding agent (e.g., an antibody), such as a human CD47 binding agent, is, for example, via diffusion, timed-release bolus, or continuous administration. In other aspects, a CD47 binding agent (e.g., an antibody) is administered directly to exposed tissue during tumor resection or other surgical procedures.

The present disclosure provides a composition, such as pharmaceutical composition, comprising a CD47 binding agent (e.g., an antibody) such as a human CD47 binding agent and a carrier (e.g., a pharmaceutically acceptable carrier). The particular carrier employed may depend on chemico-physical considerations, such as solubility and lack of reactivity with the binding agent or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). A pharmaceutical composition comprising a CD47 binding agent (e.g., an antibody) such as a human CD47 binding agent is, in one aspect, placed within containers, along with packaging material that provides instructions regarding the use of such pharmaceutical compositions. Generally, such instructions include a tangible expression describing the reagent concentration, as well as, in some embodiments, relative amounts of excipient ingredients or diluents (e.g., water, saline or PBS) that may be necessary to reconstitute the pharmaceutical composition.

In some aspects, a method described herein further comprises administering one or more additional agents, including therapeutic agents, which may be present in a composition or may be administered with a CD47 binding agent (e.g., an antibody), such as a human CD47 binding agent, or provided in a separate composition using the same or a different route of administration. The one or more additional agents, including therapeutic agents, may be administered (e.g., for combination therapy) together or separately (e.g., simultaneously, alternatively, sequencially) with a CD47 binding agent (e.g., antibody). Such additional therapeutic agents include, but are not limited to, therapeutic antibodies, immunotherapies and immunotherapeutic agents, cytotoxic agents, chemotherapeutic agents, and inhibitors.

Therapeutic antibodies that can be used with a CD47 binding agent (e.g., an antibody) as described herein (e.g., for combination therapy) include, but are not limited to, trastuzumab; abciximab; daclizumab; BEC2; IMC-C22; vitaxin; Campath 1 H/LDP-03; Smart M195; epratuzumab; bectumomab; visilizumab; CM3, a humanized anti-ICAM3 antibody; IDEC-I 14; ibritumomab tiuxetan; IDEC-131; IDEC-151; IDEC-152; SMART anti-CD3; eculizumab; adalimumab; certolizumab; IDEC-I 51; MDX-CD4; CD20-sreptdavidin; CDP571; LDP-02; OrthoClone OKT4A; ruplizumab; natalizumab; and lerdelimumab.

Immunotherapies and immunotherapeutic agents that can be used with a CD47 binding agent (e.g., an antibody) as described herein (e.g., for combination therapy) include, but are not limited to, cytokines, such as granulocyte- macrophage colony-stimulating factor (GM-CSF), granulocyte-colony stimulating factor (G-CSF), macrophage inflammatory protein (MIP)-l -alpha, interleukins (including IL-I, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund’s adjuvant (IF A); QS-21; DETOX; Levamisole; and Dinitrophenyl (DNP), and combinations thereof, such as, for example, combinations of, interleukins, for example, IL-2 with other cytokines, such as IFN-alpha. In some embodiments, the immunotherapy includes an immunotherapeutic agent that modulates immune responses, for example, a checkpoint inhibitor or a checkpoint agonist. In some embodiments, the immunotherapeutic agent is an antibody modulator that targets PD-1, PD-L1, PD-L2, CEACAM (e g., CEACAM-1, -3 and/or -5), CTLA-4, TIM-3, LAG-3, VISTA, BTLA, TIGIT, LAIR1, CD160, 2B4, TGF beta, OX40, 41 BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors.

In some embodiments, the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-I, LFA-I (CD1 Ia/CDI8), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

Cytotoxic agents that can be used with a CD47 binding agent (e.g., an antibody) as described herein (e.g., for combination therapy) include a substance that inhibits or prevents a cellular function and/or causes cell death or destruction. Exemplary cytotoxic agents include, but are not limited to, radioactive isotopes (e.g., At211, 1131, 1125, Y90, Re186, Re188, Sm153, Bi212, P32, Pb212 and radioactive isotopes of Lu); growth inhibitory agents; enzymes and fragments thereof such as nucleolytic enzymes; and toxins such as small molecule toxins or enzymatically active toxins of bacterial, fungal, plant or animal origin, including fragments and/or variants thereof. Other exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents that can be used with a CD47 binding agent (e.g., an antibody) as described herein (e.g., for combination therapy) include chemical compounds useful in the treatment of cancer. Examples of chemotherapeutic agents include, but are not limited to, erlotinib, bortezomib, disulfiram, epigallocatechin gallate, salinosporamide A, carfilzomib, 17-AAG (geldanamycin), radicicol, lactate dehydrogenase A (LDH-A), fulvestrant, sunitib, letrozole, imatinib mesylate, fmasunate, oxaliplatin, 5-FET (5-fluorouracil), leucovorin, Rapamycin, Lapatinib, Lonafamib (SCH 66336), sorafenib, Bayer Labs), gefitinib, AG1478; alkylating agents such as thiotepa and CYTOXAN®; cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; cally statin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5 alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancrati statin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma II and calicheamicin omega I (Angew Chem. Inti. Ed. Engl. 1994 33: 183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L- norleucine, doxorubicin, morpholino-doxorubicin, cyanomorpholino- doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Ore.); razoxane; rhizoxin; sizofuran; spirogermanium; tenuazonic acid; triaziquone; 2,2’,2″-trichlorotriethylamine; trichothecenes especially T-2 toxin, verracurin A, roridin A and anguidine); urethan; vindesine; dacarbazine; mannomustine; mitobronitol; mitolactol; pipobroman; gacytosine; arabinoside “Ara-C″); cyclophosphamide; thiotepa; taxoids, e.g., paclitaxel, ABRAXANE® (Cremophor-free), albumin-engineered nanoparticle formulations of paclitaxel (American Pharmaceutical Partners, Schaumberg, III.), and docetaxel/doxetaxel; chloranmbucil; gemcitabine; 6-thioguanine; mercaptopurine; methotrexate; platinum analogs such as cisplatin and carboplatin; vinblastine; etoposide (VP-16); ifosfamide; mitoxantrone; vincristine; vinorelbine; novantrone; teniposide; edatrexate; daunomycin; aminopterin; capecitabine; ibandronate; CPT-I I; topoisomerase inhibitor RFS 2000; difluorom ethyl ornithine (DMFO); retinoids such as retinoic acid; and pharmaceutically acceptable salts, acids and derivatives of any of the above. Chemotherapeutic agent also includes (i) anti-hormonal agents that act to regulate or inhibit hormone action on tumors such as anti-estrogens and selective estrogen receptor modulators (SERMs), including, for example, tamoxifen (including tamoxifen citrate), raloxifene, droloxifene, iodoxyfene, 4-hydroxytamoxifen, trioxifene, keoxifene, LY117018, onapristone, and toremifme citrate; (ii) aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; (iii) antiandrogens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LEIRTOTECAN®; ABARELIX®; and (ix) pharmaceutically acceptable salts, acids and derivatives of any of the above.

Chemotherapeutic agents also include antibodies, as described above, including alemtuzumab, bevacizumab; cetuximab; panitumumab, rituximab, pertuzumab, tositumomab, and the antibody drug conjugate, gemtuzumab ozogamicin. Additional humanized monoclonal antibodies with therapeutic potential as agents in combination with the CD47 binding agents (e.g., antibodies) described herein include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nivolumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-l2 (ABT-8744695, Wyeth Research and Abbott Laboratories) which is a recombinant exclusively human-sequence, full-length IgGl λ antibody genetically modified to recognize interleukin- 12 p40 protein. Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-l7-butyrate, hydrocortisone- 17-valerate, aclometasone dipropionate, betamethasone valerate, betamethasone dipropionate, prednicarbate, clobetasone-l 7-butyrate, clobetasol-I 7-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNF alpha) blockers such as etanercept, infliximab, adalimumab, certolizumab pegol, golimumab (Simponi), Interleukin 1 (IL-1) blockers such as anakinra, T cell costimulation blockers such as abatacept, Interleukin 6 (IL-6) blockers such as tocilizumab; Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-Ml prime; Secreted homotrimeric LTa3 and membrane bound heterotrimer LTal/l32 blockers such as Anti-lymphotoxin alpha (LTa); miscellaneous investigational agents such as thioplatin, PS-341, phenylbutyrate, ET- I8-OCH3, or famesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol); beta-lapachone; lapachol; colchicines; betulinic acid; acetylcamptothecin, scopolectin, and 9- aminocamptothecin); podophyllotoxin; tegafur; bexarotene; bisphosphonates such as clodronate, etidronate, NE-58095, zoledronic acid/zoledronate, alendronate, pamidronate, tiludronate, or risedronate; and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifamib (R11577); orafenib, ABT510; Bcl-2 inhibitor such as oblimersen sodium pixantrone; farnesyltransferase inhibitors such as lonafamib (SCH 6636); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin combined with 5-FU and leucovorin. Chemotherapeutic agents also include Poly ADP ribose polymerase (PARP) inhibitors: olaparib, rucaprib niraparib, talzoparib.

Inhibitors that can be used with a CD47 binding agent (e.g., an antibody) as described herein (e.g., for combination therapy) include, but are not limited to, kinase inhibitors such as imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilter, larotrectinib, lorlatinib, netarsudil, osimertinib, pemigatinib, pexidartinib, ribociclib, temsirolimus, XL-092, XL-147, XL-765, XL-499, and XL-880. In some embodiments, a compound as described herein can be used in combination with a HSP90 inhibitor (e.g., XL888), liver X receptor (LXR) modulators, retinoid-related orphan receptor gamma (RORy) modulators, checkpoint inhibitors such as a CK1 inhibitor or aCK1α inhibitor, a Wnt pathway inhibitor (e.g., SST-215), or a mineralocorticoid receptor inhibitor, (e.g., esaxerenone) or XL-888 for the treatment of a disease disclosed herein such as cancer. In some embodiments, a CD47 binding agent (e.g., an antibody) as disclosed herein can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, 1NS-R, IGF-1R, IR-R, PDGFαR, PDGFβ/R, CSFIR, KIT, FLK-II, KDR/FLK-1, FLK-4, flt-1, FGFR1, FGFR2, FGFR3, FGFR4, Ron, Sea, TRKA, TRKB, TRKC, FLT3, VEGFR/Flt2, Flt4, EphAl, EphA2, EphA3, EphB2, EphB4, Tie2, Src, Fyn, Lck, Fgr, Btk, Fak, SYR, FRK, JAK (JAK1 and or JAK2), ABL, ALK, CDK7, CDK12, KRAS, and B-Raf. Additional non-limiting examples of inhibitors include an FGFR inhibitor (FGFR1, FGFR2, FGFR3 or FGFR4, e.g., pemigatinib, an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g., bevacizumab, pazopanib, sunitinib, sorafenib, axitinib, regorafenib, ponatinib, vandetanib, ramucirumab, lenvatinib, ziv-aflibercept), a PARP inhibitor (e.g. olaparib, rucaparib, veliparib or niraparib), a JAK inhibitor (e.g., ruxolitinib, baricitinib, itacitinib), an IDO inhibitor (e.g., epacadostat, NLG919, or BMS-986205, MK7162), an LSD1 inhibitor, a TDO inhibitor, a Pl3K-delta inhibitor (e.g., parsaclisib), a Pl3K-gamma inhibitor such as Pl3K-gamma selective inhibitor, a Pim inhibitor, a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP½ phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors, or combinations thereof.

In some embodiments, a CD47 binding agent (e.g., an antibody) as disclosed herein can be used in combination with inhibitors of PD-1 or inhibitors of PD-L1, e.g., an anti-PD-1 monoclonal antibody or an anti-PD-L1 monoclonal antibody, for example, nivolumab (Opdivo), pembrolizumab (Keytruda, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZM009, AK105, HLX10, MGA012, PDR001, TSR-042, AMP-224, AMP- 514, PDR001, durvalumab, pidilizumab (Imfinzi®, CT-011), CK-301, BMS 936559, and MPDL3280A.

In some embodiments, a PD-L1 binding agent (e.g., an antibody) as disclosed herein can be used in combination with inhibitors of PD-1 or inhibitors of PD-L1, e.g., an anti-PD-1 monoclonal antibody or an anti-PD-L1 monoclonal antibody, for example, nivolumab (Opdivo), pembrolizumab (Keytruda, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZM009, AK105, HLX10, and TSR-042, AMP-224, AMP- 514, PDR001, durvalumab, pidilizumab (Imfinzi®, CT-011), CK-301, BMS 936559, MPDL3280A, tislelizumab, BMS-935559, MEDI4736, FAZ053, KN035, CS1001, CBT-502, A167, STI-A101, BGB-A333, MSB-2311, HLX20, AUNP12, CA-170, BMS-986189, LY3300054, and MSB0010718C.

In some embodiments, a CD47 binding agent (e.g., an antibody) as disclosed herein can be used in combination with CTLA-4 inhibitors, e.g., an anti-CTLA-4 antibody, for example, ipilimumab (Yervoy), tremelimumab and AGEN1884, or with phosphatidylserine inhibitors, for example, bavituximab (PGN401), or with antibodies to cytokines (IL-10, TGF-b, and the like), or with bispecific antibodies that bind to PD-L1 and CTLA-4 (e.g., AK104) or PD-1 and CTLA-4, or with other anti-cancer agents such as cemiplimab.

The additional agent may be a pharmaceutically acceptable salt, ester, amide, hydrate, and/or prodrug of any of these therapeutic agents described above or other agents.

The additional therapeutic agent may be a pharmaceutically acceptable salt, ester, amide, hydrate, and/or prodrug of any of these therapeutic agents described above or other agents.

It is understood that modifications which do not substantially affect the activity of the various embodiments described herein are also provided within the definition of the subject matter described herein. Accordingly, the following examples are intended to illustrate but not limit the present disclosure.

### EXAMPLES

### Example 1: Antibody Generation

To obtain binders for human CD47, antibody discovery was conducted by phage display of human Fab libraries and was carried out using standard protocols. The extracellular domain of human CD47 was purchased from Acro Biosystems (biotinylated human CD47 His-Avitag Acro Cat No. CD7-H82E9 and human CD47 His-tag Acro Cat No. CD7-5227). The non-biotinylated extracellular domain of CD47 was biotinylated using EZ-Link NHS-PEG₁₂-Biotin (ThermoScientific Cat. No. 21312) using standard protocol as needed. Phage clones were screened for the ability to bind to biotinylated human CD47. Briefly, Fab-formatted phage libraries were constructed using expression vectors capable of replication and expression in phage (also referred to as a phagemid). Both the heavy chain and the light chain were encoded in the same expression vector, where the heavy chain was fused to a truncated variant of the phage coat protein plll. The light chain and heavy chain-pill fusion were expressed as separate polypeptides and assembled in the bacterial periplasm, where the redox potential enables disulfide bond formation, to form the antigen binding domain (Fab) of the candidate antibody.

The library was created using sequences derived from a specific human heavy chain variable domain (VH3-23) and a specific human light chain variable domain (Vk-1). Light chain variable domains within the screened library were generated with diversity was introduced into the VL CDR3 (L3) and where the light chain VL CDR1 (L1) and CDR2 (L2) remained the human germline sequence. For the screened library, all three CDRs of the VH domain were diversified to match the positional amino acid frequency by CDR length found in the human antibody repertoire. The phage display heavy chain (SEQ ID NO:92) and light chain (SEQ ID NO:93) scaffolds used in the library are listed below, where a lower case “x” represents CDR amino acids that were varied to create the library, and bold italic represents the CDR sequences that were constant.

The sequence for SEQ ID NO:92 was

EVQLVESGGGLVQPGGSLRLSCAASGFTFSXXXXXWVRQAPGKGLEWVAX XXXXXXXXXXXXXXXXRFTISADTSKNTAYLQMNSLRAEDTAVYYCARXX XXXXXXXXXXXXWGQGTLVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSC. The

sequence for SEQ ID NO:93 was

DIQMTQSPSSLSASVGDRVTITCRASQSVSSAVA WYQQKPGKAPKLLIYS ASSLYS GVPSRFSGSRSGTDFTLTISSLQPEDFATYYCXXXXXXXXXFGQ
GTKVEIKRTVAAPSVFIFPPSDSQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Diversity was created through mutagenesis using degenerate DNA oligonucleotide primers to introduce diversity into VL CDR3 and VH CDR1 (H1), CDR2 (H2) and CDR3 (H3) to mimic the diversity found in the natural antibody repertoire, as described in more detail in Kunkel, TA (PNAS Jan. 1, 1985. 82 (2) 488-492), herein incorporated by reference in its entirety. Briefly, uracil-incorporated single-stranded circular DNA were prepared from isolated phage using standard procedures and Kunkel mutagenesis carried out to introduce diversity to the four CDRs. Chemically-synthesized DNA was then electroporated into TG1 cells, followed by recovery. Recovered cells were sub-cultured and infected with M13K07 helper phage to produce the phage library.

Phage panning was performed using standard procedures. Briefly, the first round of phage panning was performed with target immobilized on streptavidin magnetic beads which were subjected to approximately 1×10¹² phages from the prepared library in a volume of 1 mL in PBST-2% BSA. After a one-hour incubation, the bead-bound phage were separated from the supernatant using a magnetic stand. Beads were washed three times to remove non-specifically bound phage and were then added to ER2738 cells (5 mL) at OD₆₀₀ of approximately 0.6. After 20 minutes incubation at room temperature, infected cells were sub-cultured in 25 mL 2xYT + Ampicillin and M13K07 helper phage (final concentration of approximately 1×10¹⁰ pfu/ml) and allowed to grow overnight at 37° C. with vigorous shaking. The next day, phage were prepared using standard procedures by PEG precipitation. Pre-clearance of phage specific to SAV-coated beads was performed prior to panning. The second round of panning was performed using the KingFisher magnetic bead handler with 50 or 100 nM bead-immobilized CD47 antigen using standard procedures (100 nM CD47 for round 3, 50 nM CD47 for round 4). In total, 3-4 rounds of phage panning were performed to enrich in phage displaying Fabs specific for the target antigen. Target-specific enrichment was confirmed using polyclonal ELISA and individual clones were isolated and further verified by performing monoclonal phage ELISA. DNA sequencing was used to determine the sequence of the CDRs of isolated Fab clones containing a candidate antibodies.

The genes encoding heavy chain and light chain variable domains of the candidate antibodies were cloned separately into mammalian expression vectors for expression as full length IgGs in mammalian cells.

For the full length IgGs, the heavy chain constant regions (e.g., CH1 = regular text; Hinge = italicized text; CH2 = bold text; and CH3 = underline text) included the following amino acid sequence:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS HEDPEVKFNWYVDGVEVHNAKTKPREEQYNSTYRVVSVLTVLHQDWLNGK EYKCKVSNKALPAPIEKTISKAKGQPREPQVYTLPPSRDELTKNQVSLTC LVKGFYPSDIAVEWESNGQPENNYKTTPPVLDSDGSFFLYSKLTVDKSRW QQGNVFSCSVMHEALHNHYTQKSLSLSPGK (SEQ ID NO:94).

For the full length IgGs, the light chain constant region (e.g., CL) included the following amino acid sequence:

RTVAAPSVFIFPPSDSQLKSGTASVVCLLNNFYPREAKVQWKVDNALQSG
NSQESVTEQDSKDSTYSLSSTLTLSKADYEKHKVYACEVTHQGLSSPVTK
SFNRGEC (SEQ IDNO:95).

The IgG antibodies were purified from culture supernatant using Protein A resin.

### Example 2: Screening and Selection

Antibodies to CD47 were generated by phage display, for example, such as described in Example 1. For example, to determine qualitative binding, bio-layer interferometry (BLI) was used to confirm the specific interaction of the antigens to the candidate antibodies obtained in Example 1.

The bivalent interaction of binders to biotinylated human CD47 (see Example 1) immobilized on a streptavidin biosensor was monitored using an Octet (Pall ForteBio) instrument. For identification of weak binders, to increase affinity due to avidity, Fc tagged version (Acro Cat. No. CD7-H5256 (Fc-tag)) was used for Octet assays. Soluble antigens were then added to the system and binding was measured.

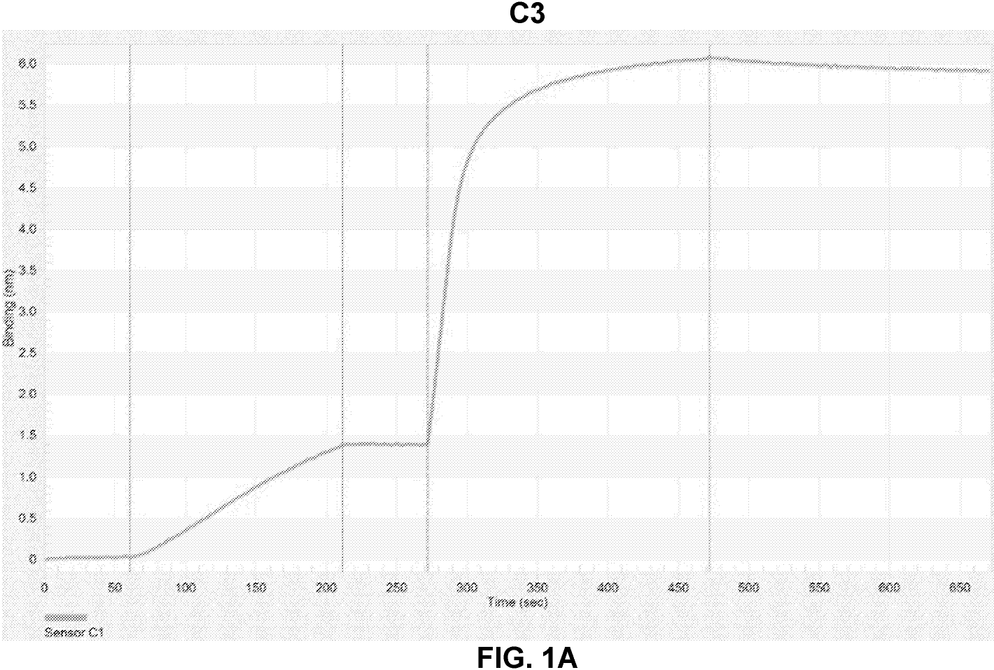

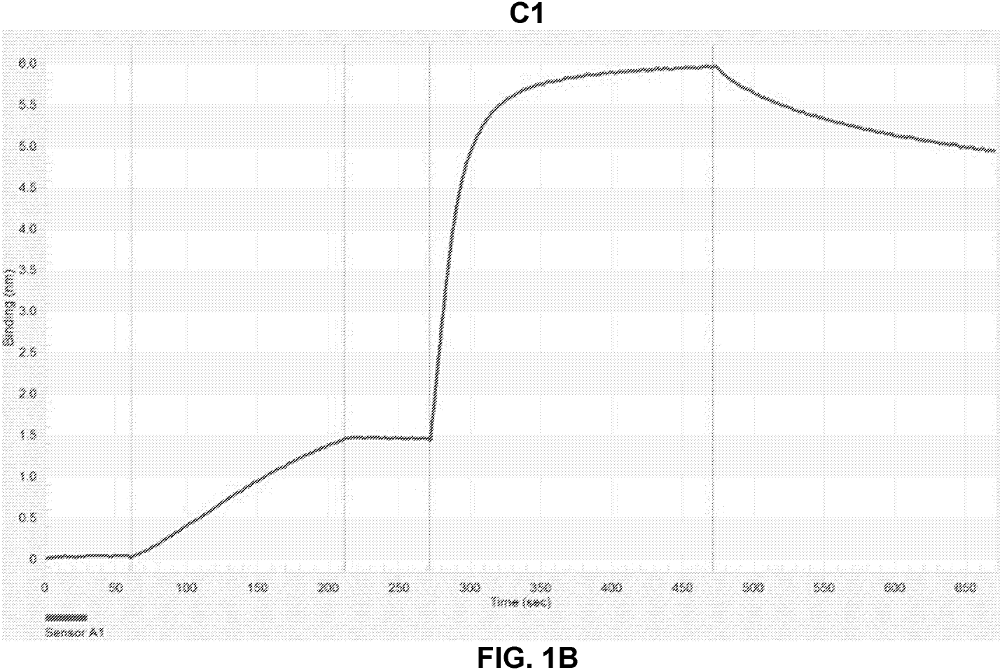

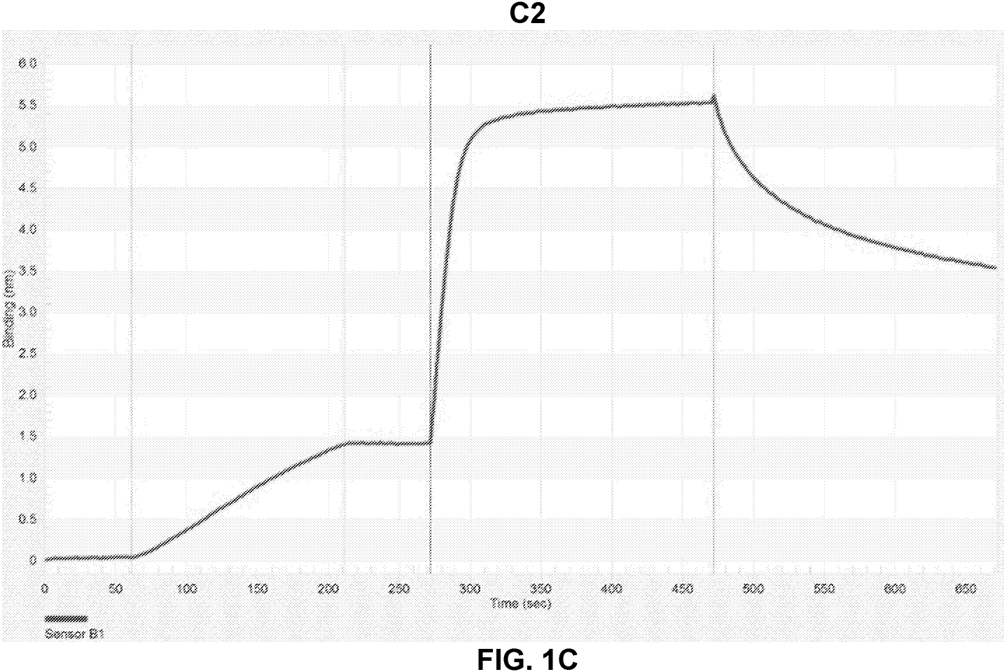

Exemplary sensograms for antibody binding are shown in FIGS. 1A-1C. Qualitative binding affinity results are shown in Table 4. For qualitative binding affinity using Octet, strong binding is indicated by the symbol “+++”, moderate binding is indicated by “++”, and weak binding is indicated by “+”. “ND” indicates that antibody binding was not determined. Results show that 15 antibodies showed strong binding affinity (C3, C4, C5, C8, C12, C20, C23, C25, C27, C29, C33, C36, C49, C54, and C57), 14 antibodies showed moderate cell binding affinities (C1, C11, C21, C22, C35, C38, C40, C43, C46, C51, C52, C53, C55, and C56), and 24 antibodies showed weak cell binding affinities (C2, C6, C9, C15, C18, C19, C24, C26, C28, C30, C31, C34, C37, C39, C41, C42, C45, C47, C48, C58, C59, C60, C61, and C64).

TABLE 4

Qualitative Binding Affinity by Octet

| | |
|---|---|
| Binder | Qualitative Binding Affinity (Octet) |
| C1 | ++ |
| C2 | + |
| C3 | +++ |
| C4 | +++ |
| C5 | +++ |
| C6 | + |
| C7 | ND |
| C8 | +++ |
| C9 | + |
| C10 | ND |
| C11 | ++ |
| C12 | +++ |
| C13 | ND |
| C14 | ++ |
| C15 | + |
| C16 | ++ |
| C17 | ND |
| C18 | + |
| C19 | + |
| C20 | +++ |
| C21 | ++ |
| C22 | ++ |
| C23 | +++ |
| C24 | + |
| C25 | +++ |
| C26 | + |
| C27 | +++ |
| C28 | + |
| C29 | +++ |
| C30 | + |
| C31 | + |
| C32 | ND |
| C33 | +++ |
| C34 | + |
| C35 | ++ |
| C36 | +++ |
| C37 | + |
| C38 | ++ |
| C39 | + |
| C40 | ++ |
| C41 | + |
| C42 | + |
| C43 | ++ |
| C44 | ++ |
| C45 | + |
| C46 | ++ |
| C47 | + |
| C48 | + |
| C49 | +++ |
| C50 | ND |
| C51 | ++ |
| C52 | ++ |
| C53 | ++ |
| C54 | +++ |
| C55 | ++ |
| C56 | ++ |
| C57 | +++ |
| C58 | + |
| C59 | + |
| C60 | + |
| C61 | + |
| C62 | ND |
| C63 | ND |
| C64 | + |

Binding to mouse CD47 (mCD47) was characterized by assessing cell binding to mouse cells. Specifically, binding of the antibodies to mouse cells was determined by flow cytometry. Antibodies were incubated with the indicated cell lines at the indicated concentrations followed by labeling with a fluorescently labeled secondary antibody. Mouse CD47 was obtained from Acro Biosystems (Acro Cat. No. CD7-M82E4, Acro Cat. No. CD7-M522b) and biotinylated using standard protocols. The free biotin was removed by extensive dialysis against PBS. Biotinylated antigen (CD47) was immobilized on a streptavidin sensor. The cross-reactive antibodies were identified by the association to mouse CD47 using Octet.

Mouse cross-reactivity results are shown in Table 5. Mouse cross-reactivity is indicated with a “Y,” and no mouse cross-reactivity is indicated with a “X.” Antibody cross-reactivity that was not determined is indicated with a “ND.”

TABLE 5

Mouse CD47 Cross-Reactivity

| | |
|---|---|
| Binder | mCD47 Cross-Reactive |
| C1 | X |
| C2 | X |
| C3 | X |
| C4 | Y |
| C5 | Y |
| C6 | X |
| C7 | X |
| C8 | X |
| C9 | X |
| C10 | X |
| C11 | X |
| C12 | Y |
| C13 | ND |
| C14 | X |
| C15 | X |
| C16 | X |
| C17 | ND |
| C18 | X |
| C19 | X |
| C20 | Y |
| C21 | X |
| C22 | X |
| C23 | Y |
| C24 | X |
| C25 | X |
| C26 | X |
| C27 | Y |
| C28 | X |
| C29 | Y |
| C30 | X |
| C31 | X |
| C32 | X |
| C33 | Y |
| C34 | X |
| C35 | Y |
| C36 | X |
| C37 | X |
| C38 | Y |
| C39 | X |
| C40 | Y |
| C41 | X |
| C42 | ND |
| C43 | X |
| C44 | X |
| C45 | X |
| C46 | X |
| C47 | X |
| C48 | X |
| C49 | X |
| C50 | ND |
| C51 | ND |
| C52 | X |
| C53 | Y |
| C54 | Y |
| C55 | X |
| C56 | ND |
| C57 | Y |
| C58 | X |
| C59 | X |
| C60 | X |
| C61 | X |
| C62 | ND |
| C63 | ND |
| C64 | X |

### Example 3: Additional Screening and Selection

Antibodies that were selected for binding to CD47, for example, such as those described in Example 2, were evaluated for binding to cells that express CD47. For example, antibodies were tested using flow cytometry for binding to MDA-MB-231 cells (American Type Culture Collection “ATCC” (ATCC-HTB-26))), which have a surface CD47 copy number of approximately 500,000.

Cells were harvested at 70-90% confluence on the day of the assay. Cells were collected by centrifugation at 200xg for 5 minutes and media was removed. Cells were resuspended at 2×10⁶ cells per mL in cold PBS. An 8-point antibody dilution series (2x concentration) was prepared in PBS to cover the expected binding affinities of the antibodies being tested. 50 µL per well of the antibody dilution was plated in a 96 well V-bottom plate (Costar 3897). 50 µL per well of cell suspension was added. Plates were placed at 4° C. for 45-60 minutes.

Cells were collected by centrifugation at 400xg for 7 minutes and primary antibody was removed. 50 µL per well of AF488 goat anti human IgG Fab (Jackson Immuno Research 109-547-003) at 1:100 dilution was added. Plates were placed at 4° C. for 30 minutes.

Cells were collected by centrifugation at 400xg for 7 minutes and secondary antibody was removed. Cells were resuspended in 50 µL per well of PBS and analyzed by flow cytometry. Binding curves were calculated using the mean fluorescence intensity (MFI) of the FITC fluorescence signal on the cells.

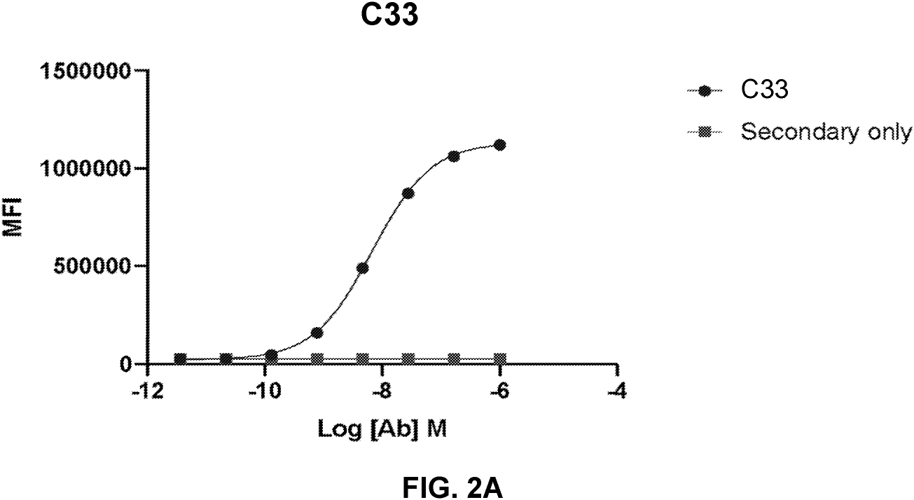

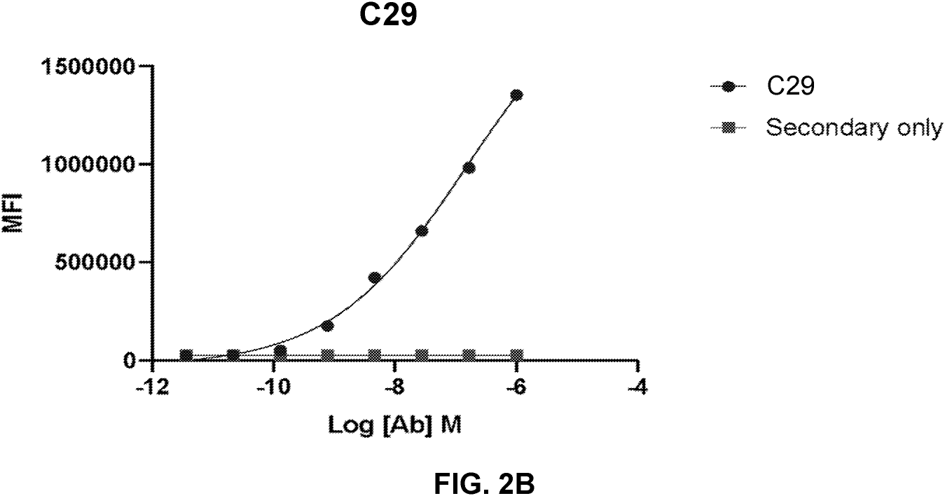

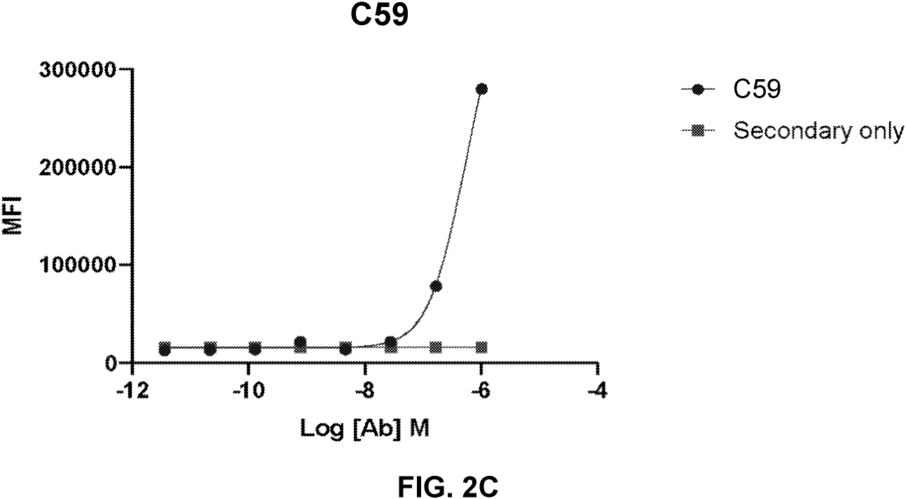

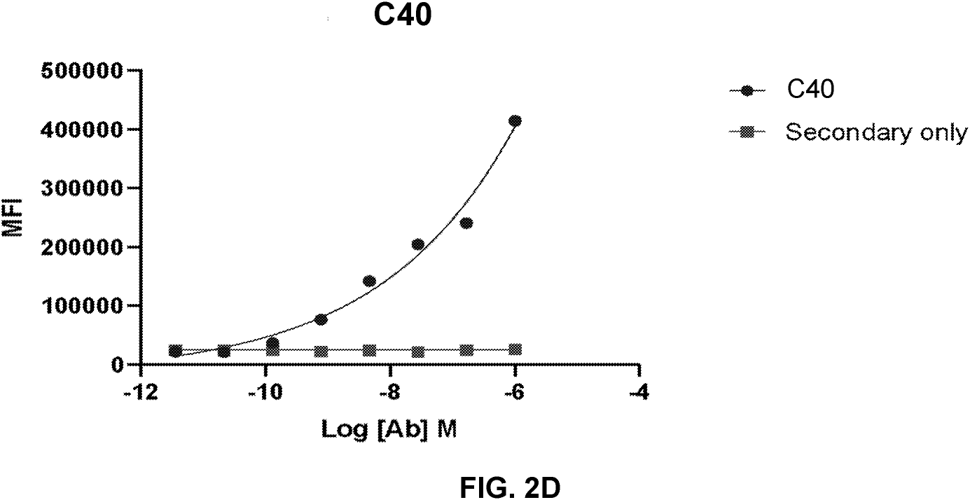

Exemplary binding curves are depicted in FIGS. 2A-2D. Qualitative binding affinity to cells and half maximal effective concentration (EC₅₀) of cell binding for the assayed antibodies are summarized in Table 6. For qualitative binding affinity, strong binding, < 2E-08 M (< 20 nM), is indicated by the symbol “++++”, moderate binding, 2E-08 - 2E-07 M (20 - 200 nM), is indicated by “+++”, moderate/weak binding, 2E-07 M -1E-06 M (200 - 1000 nM), is indicated by “++”, and weak binding, > 1E-06 M (> 1000 nM), is indicated by “+”. “ND” indicates that antibody binding was not determined. Results show that 10 antibodies showed strong cell binding affinity (C3, C5, C8, C9, C20, C21, C25, C27, C33, and C36), 12 antibodies showed moderate cell binding affinity (C2, C6, C12, C18, C22, C28, C29, C30, C31, C32, C48, and C54), 15 antibodies showed moderate/weak binding C15, C19,C34, C37, C42, C44, C45, C46, C47, C49, C55, C56, C58, C59, and C64), and 21 antibodies showed weak binding (C1, C4, C10, C11, C14, C16, C23, C24, C26, C35, C38, C39, C40, C41, C43, C51, C52, C53, C57, C60 and C61).

TABLE 6

Qualitative Binding Affinity and EC₅₀ of Antibody Binding to MDA-MB-231 Cells

| | | |
|---|---|---|
| Binder | Qualitative Binding Affinity (Cells) | EC50 (M) |
| C1 | + | - 0.006233 |
| C2 | +++ | 6.78E-08 |
| C3 | ++++ | 1.38E-09 |
| C4 | + | ~ 4.032e-006 |
| C5 | ++++ | 2.56E-10 |
| C6 | +++ | 5.27E-08 |
| C7 | ND | ND |
| C8 | ++++ | 3.12E-10 |
| C9 | ++++ | ~ 9.230e-009 |
| C10 | + | ~ 4.753e-006 |
| C11 | + | 5.54E-06 |
| C12 | +++ | 7.13E-08 |
| C13 | ND | ND |
| C14 | + | 3.54E-06 |
| C15 | ++ | 3.95E-07 |
| C16 | + | ~ 0.0001529 |
| C17 | + | 4.52E-03* |
| C18 | ++ | 2.93E-08 |
| C19 | ++ | 9.42E-07 |
| C20 | ++++ | 8.46E-10 |
| C21 | ++++ | 7.63E-09 |
| C22 | +++ | 2.73E-08 |
| C23 | + | 1.53E-06 |
| C24 | + | 1.09E-06 |
| C25 | ++++ | 5.77E-10 |
| C26 | + | ~ 1.143 |
| C27 | ++++ | 5.82E-10 |
| C28 | +++ | 1.47E-07 |
| C29 | +++ | 1.57E-07 |
| C30 | +++ | 1.11E-07 |
| C31 | +++ | 1.28E-07 |
| C32 | +++ | 1.08E-07 |
| C33 | ++++ | 6.76E-09 |
| C34 | ++ | 2.60E-07 |
| C35 | + | ~ 4.667 |
| C36 | ++++ | 2.13E-09 |
| C37 | ++ | 3.64E-07 |
| C38 | + | 1.08E-05 |
| C39 | + | 1.89E-06 |
| C40 | + | ~ 919343207 |
| C41 | + | 4.60E-06 |
| C42 | ++ | 8.56E-07 |
| C43 | + | 3.99E-06 |
| C44 | ++ | 2.46E-07 |
| C45 | ++ | 6.16E-07 |
| C46 | ++ | 2.25E-07 |
| C47 | ++ | 5.32E-07 |
| C48 | +++ | 1.78E-07 |
| C49 | ++ | 5.32E-07 |
| C50 | ND | ND |
| C51 | + | 1.01E-01 |
| C52 | + | 2.31E-04 |
| C53 | + | ~ 0.0006719 |
| C54 | +++ | 1.83E-07 |
| C55 | ++ | 8.97E-07 |
| C56 | ++ | 2.76E-07 |
| C57 | + | 1.14E-04 |
| C58 | ++ | 7.52E-07 |
| C59 | ++ | 5.50E-07 |
| C60 | + | 7.42E-03 |
| C61 | + | ~ 19.63 |
| C62 | ND | ND |
| C63 | ND | ND |
| C64 | ++ | 3.64E-07 |

*this is blank in data table but should be weak

### Example 4: Functional Assays

Antibodies that were selected for binding to CD47, for example, such as those described in Examples 2 and 3, were evaluated for inhibition of a CD47/SIRPα signaling in cell types expressing CD47, including JeKo-1 cells (ATCC (ATCC CRL-3006)), which have a surface copy number of CD47 of approximately 50,000.

To test the ability of antibodies to block CD47/SIRPα signaling, antibodies were assayed using a CD47/SIRPα Signaling Bioassay Kit (93-1135C19, Eurofins DiscoverX) using the manufacturer protocol.

The signaling assays are engineered to co-express a ProLink™ (PK) tagged immune checkpoint receptor and an Enzyme Acceptor (EA) tagged SH2 domain. Ligand engagement leads to receptor activation and phosphorylation, resulting in SH2-EA recruitment to the receptor, and forcing complementation of the two β-galactosidase enzyme fragments (EA and PK). The resulting functional enzyme hydrolyzes substrate to generate a chemiluminescent signal. Blocking of the ligand engagement leads to a drop in chemiluminescent signal.

Briefly, 30,000 target cells were added to wells in 40 µL Cell Plating Reagent in White Bottom 96-well Plates (Corning 3917). Antibody dilutions were prepared in cell plating reagent and 20 µL of antibody dilution were added to cells. Next, 10,000 freshly thawed SIRPα Jurkat cells in 40 µL Cell Plating Reagent were added to each well. Plates were incubated for 24 hours at 37° C. Following incubation, 10 µL BioAssay Reagent 1 was added to wells and plates were incubated for 15 minutes at room temp in the dark. Then, 40 µL BioAssay Reagent 2 was added to wells and plates were incubated for 1 hour at room temperature in the dark. Plates were read on a ClarioStar Plate Reader (BMG Labtech).

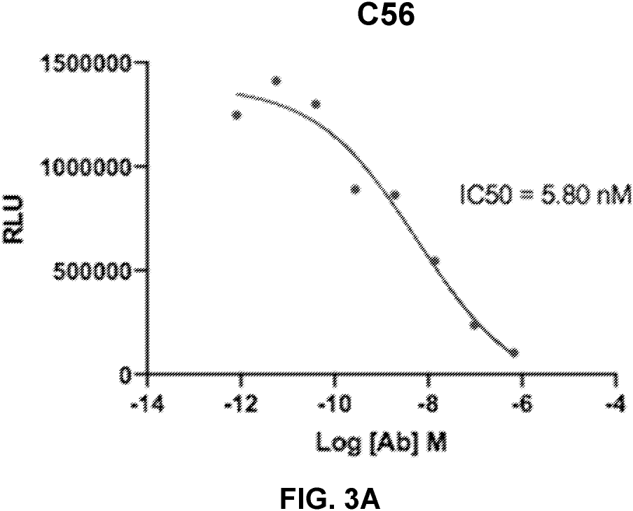

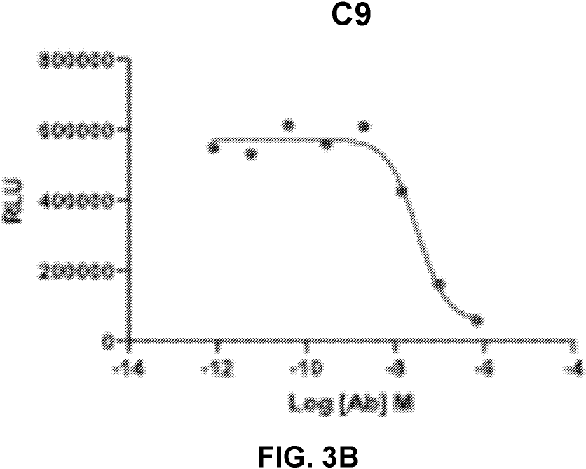

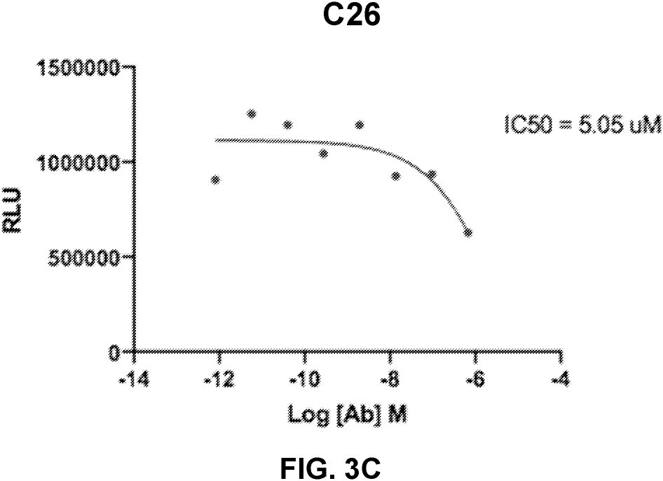

Exemplary results of the CD47/SIRPα checkpoint signaling assay are shown in FIGS. 3A-3C. IC₅₀ values for assays in JeKo cells are shown in Table 7. Strong blocking, < 2E-08 M (< 20 nM), is indicated by the symbol “+++”, moderate blocking, 2E-08 - 2E-07 M (20 - 200 nM), is indicated by “++”, and weak blocking, > 2E-07 M (> 200 nM), is indicated by “+”. “ND” indicates that interaction blocking was not determined. Results show that 17 antibodies had strong blocking of the CD47/SIRPα interaction (C5, C12 C20, C21, C22, C23, C25, C27, C29, C33, C38, C40, C52, C54, C55, C56, and C57), 15 antibodies had moderate blocking (C1, C2, C9, C15, C19, C31, C39, C41, C43, C46, C51, C53, C58, C59, and C64), and 7 antibodies had weak or no blocking (C18, C24, C26, C34, C37, C42, and C60).

TABLE 7

CD47/SIRPα Interaction Blocking Reporter Assay

| | | |
|---|---|---|
| Binder | CD47/SIRPα Interaction Blocking Reporter Assay | IC50 (M) |
| C1 | ++ | 4.38E-08 |
| C2 | ++ | 9.30E-08 |
| C3 | ND | ND |
| C4 | ND | ND |
| C5 | +++ | 3.94E-10 |
| C6 | ND | ND |
| C7 | ND | ND |
| C8 | ND | ND |
| C9 | ++ | 3.10 E-08 |
| C10 | ND | ND |
| C11 | ND | ND |
| C12 | +++ | 4.80E-10 |
| C13 | ND | ND |
| C14 | ND | ND |
| C15 | ++ | 9.20E-08 |
| C16 | ND | ND |
| C17 | ND | ND |
| C18 | + | No Blocking |
| C19 | ++ | 1.50E-07 |
| C20 | +++ | 9.24E-11 |
| C21 | +++ | 2.89E-09 |
| C22 | +++ | 5.90E-09 |
| C23 | +++ | 5.60E-09 |
| C24 | + | 3.50E-06 |
| C25 | +++ | 4.20E-11 |
| C26 | + | 5.10E-06 |
| C27 | +++ | 4.80E-10 |
| C28 | ND | ND |
| C29 | +++ | 7.86E-10 |
| C30 | ND | ND |
| C31 | ++ | 1.41E-07 |
| C32 | ND | ND |
| C33 | +++ | 5.50E-10 |
| C34 | + | No Blocking |
| C35 | ++ | 6.20E-08 |
| C36 | ND | ND |
| C37 | + | No Blocking |
| C38 | +++ | 2.35E-09 |
| C39 | ++ | 1.23E-07 |
| C40 | +++ | 5.38E-09 |
| C41 | ++ | 2.10E-08 |
| C42 | + | No Blocking |
| C43 | ++ | 1.60E-07 |
| C44 | ND | ND |
| C45 | ND | ND |
| C46 | ++ | 1.28E-07 |
| C47 | ND | ND |
| C48 | ND | ND |
| C49 | ND | ND |
| C50 | ND | ND |
| C51 | ++ | 2.30E-08 |
| C52 | +++ | 7.17E-09 |
| C53 | ++ | 9.50E-08 |
| C54 | +++ | 1.68E-10 |
| C55 | +++ | 4.10E-09 |
| C56 | +++ | 5.80E-09 |
| C57 | +++ | 2.20E-10 |
| C58 | ++ | 8.40E-08 |
| C59 | ++ | 1.40E-07 |
| C60 | + | 3.90E-07 |
| C61 | ND | ND |
| C62 | ND | ND |
| C63 | ND | ND |
| C64 | ++ | 1.70E-07 |

### Example 5: Additional Functional Assays

Antibodies that were selected for binding to CD47, for example, such as those described in Examples 2 and 3, were evaluated for their effect in blocking interaction of CD47 ligand with SIRPα receptor and in promoting phagocytosis. For example, the effect of antibodies on target cell phagocytosis by phagocytic THP-1 effector cells (M0 macrophage-like) was tested. JeKo target cells were labeled with pHrodo Red Cell Labeling Kit for Flow Cytometry (A10026, ThermoFisher Scientific). Labeled target cells were then incubated with antibodies to allow binding to cell surface CD47. Antibody-bound labeled cells were co-cultured with phagocytic THP-1 effector cells (M0 macrophage-like) in the Incucyte live cell imaging system (Satrotius) and phase and fluorescent images taken every 30 minutes for 24 hours.

To prepare phagocytic THP-1 effector cells for the assay, THP-1 cells were grown to 0.2-0.4 million cells/ml density in complete RPMI media (10% FBS, 1X Penn-Strep) supplemented with 0.05 mM beta-ME. Cells were passed when cell density reached approximately 1 million cells/ml (every 4-5 days). 50,000 THP-1 cells were plated per well with RPMI Complete media, 0.05 mM beta-ME and 100 nM PMA in a half-area clear bottom black plate. Plates were incubated for 72 hours at 37° C., 5% CO2.

10 million Jeko target cells were suspended in Incucyte pHrodo labeling buffer (D) at a density of 1 million cells/ml and 100 ng/ml Incucyte pHrodo red cell labeling dye (component A) was added, and cells were incubated for 1 hour at 37° C. Cells were centrifuged at 200xg for 5 minutes and supernatant discarded. Cells were resuspended in excess of complete JeKo-1 media to quench excess pHrodo-red dye and incubated at room temperature for 30-60 minutes. Cells were again centrifuged, the cell pellet resuspended in 20 mL of complete RPMI medium and passed through a cell strainer to remove any clumps.

150 µL cells/well (75,000 cells per well) was added to wells of a 96 well round bottom plate. 150 µL 2x antibody dilution series was added to each well and plates incubated for 30 min at room temperature in the dark.

Media was removed from the phagocytic THP-1 effector cells, and the pHrodo-red dye labeled, CD47 bound JeKo cells added to the effector THP-1 cells (100 µL per well).

As a signal appropriation control, wells with only pHrodo-red dye labelled target cells were also analyzed. As an experiment negative control, wells with only pHrodo-red dye labelled target cells and THP-1 cells without anti-CD47 antibody was tested.

IncuCyte analysis was conducted using red channel and, optionally, phase channel, and 4 images taken per well every 30 minutes for 24 hours. The red scale was manually adjusted such that pHrodo-red labeled target cells alone wells showed no signal. The 18 µm cell size threshold was used and the minimum mean fluorescence intensity cut-off was adjusted based on the negative and positive controls at 0 hour and 5 or 6 hour time points.

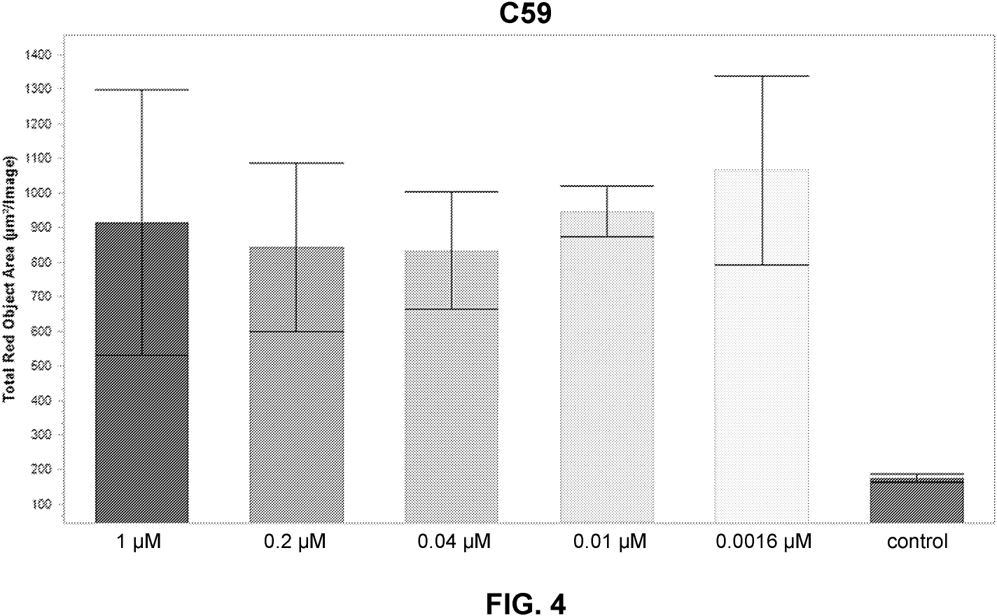

Exemplary results of the phagocytosis assay are shown in FIG. 4. Qualitative results for phagocytosis assay are shown in Table 8. For qualitative results, phagocytosis resulting from CD47/SIRPα interaction blocking in the presence of tested was compared to phagocytosis with no antibody present. Strong phagocytosis is indicated by the symbol “+++”, moderate phagocytosis is indicated by “++”, and weak phagocytosis is indicated by “+”. “ND” indicates that antibody binding was not determined. Results show that 22 antibodies showed strong CD47/SIRPα interaction blocking in the phagocytosis assay (C1, C2, C5, C9, C18, C20, C23, C29, C31, C33, C34, C35, C37, C38, C39, C41, C51, C53, C57, C58, C59, and C64), 18 antibodies showed moderate blocking (C12, C15, C19, C21, C22, C24, C25, C26, C27, C40, C42, C43, C46, C52, C54, C55, C56, and C60), and 18 antibodies showed weak blocking (C3, C4, C6, C7, C8, C10, C11, C14, C16, C17, C30, C32, C36, C45, C47, C48, C49, and C61). Thus, a number of assayed antibodies efficiently blocked the engagement of CD47 ligand on the SIRPα receptor, promoting phagocytosis.

TABLE 8

Phagocytosis Assay

| | |
|---|---|
| Binder | Phagocytosis Assay |
| C1 | +++ |
| C2 | +++ |
| C3 | + |
| C4 | + |
| C5 | +++ |
| C6 | + |
| C7 | + |
| C8 | + |
| C9 | +++ |
| C10 | + |
| C11 | + |
| C12 | ++ |
| C13 | ND |
| C14 | + |
| C15 | ++ |
| C16 | + |
| C17 | + |
| C18 | +++ |
| C19 | ++ |
| C20 | +++ |
| C21 | ++ |
| C22 | ++ |
| C23 | +++ |
| C24 | ++ |
| C25 | ++ |
| C26 | ++ |
| C27 | ++ |
| C28 | ND |
| C29 | +++ |
| C30 | + |
| C31 | +++ |
| C32 | + |
| C33 | +++ |
| C34 | +++ |
| C35 | +++ |
| C36 | + |
| C37 | +++ |
| C38 | +++ |
| C39 | +++ |
| C40 | ++ |
| C41 | +++ |
| C42 | ++ |
| C43 | ++ |
| C44 | ND |
| C45 | + |
| C46 | ++ |
| C47 | + |
| C48 | + |
| C49 | + |
| C50 | +++ |
| C51 | +++ |
| C52 | ++ |
| C53 | X |
| C54 | ++ |
| C55 | ++ |
| C56 | ++ |
| C57 | +++ |
| C58 | +++ |
| C59 | +++ |
| C60 | ++ |
| C61 | + |
| C62 | +++ |
| C63 | ND |
| C64 | +++ |

### Example 6: Red Blood Cell Binding Assay

Antibodies that were selected for binding to CD47, for example, such as those described in Examples 2 and 3, were evaluated for their binding to red blood cells. Anti-CD47 antibodies may cause red blood cell agglutination, which limits their therapeutic applications. Antibodies disclosed herein were tested to determine their effect on red blood cell agglutination using a red blood cell binding assay.

100,000 fresh human erythrocyte cells (SER-10MLRBC-SDS, Zen-Bio) were transferred to each well of a V-bottom plate. Plates were centrifuged at 500xg for 5 minutes at room temperature and supernatant was discarded. Antibodies were diluted in BD Stain Buffer (BD Biosciences) and transferred 100 µL to each well of the plate. Plates were incubated for 45 minutes at 4° C. Following incubation, 100 µL PBS was added to each well, plates were centrifuged at 500xg for 5 minutes at room temperature, and supernatant was discarded.

Next, 100 µL anti-human IgG Fab Alexa Fluor 488 (Jackson Immuno Research 109-547-003) diluted at 1:100 in BD Stain Buffer was added to each well. Plates were incubated for 45 minutes at 4° C., centrifuged at 500xg for 5 minutes at room temperature, and supernatant was discarded. Cells were washed once with 200 µL/well PBS and resuspended in 50 µL/well PBS. Samples were analyzed by flow cytometry on a Sartorius iQue Screener Plus instrument (Sartorius).

All tested antibodies resulted in no red blood cell binding compared to a positive control (data not shown).

### Example 7: Developability Assays

Antibodies that were selected for binding to CD47, for example, such as those described in Examples 2 and 3, were tested in various developability methods. For example, various chromatographic methods, including size exclusion chromatography (SEC), hydrophobic interaction chromatography (HIC), and standup monolayer adsorption chromatography (SMAC) were employed to assess developability factors, such as monomer percentage, solubility, and antibody aggregation or precipitation.

Size exclusion chromatography (SEC) analysis was performed using a 7.8 mm ID x 30 cm TSKgel G3000SWXL column (Tosoh Bioscience LLC, PN 08541) on an Agilent 1100 HPLC. Antibodies were normalized to 1 mg/mL concentration in Dulbecco’s PBS (pH 7.4, without Ca2+/Mg2+) and clarified via centrifugation to pellet particulates while still retaining soluble aggregates. The mobile phase buffer was Dulbecco’s PBS (pH 7.4, without Ca2+/Mg2+). For each sample, 10 µL was loaded and isocratically eluted at 1.0 mL/min over 20 minutes. Absorbance was monitored at 280 nm. Chromatographic peaks were integrated to determine % homogeneity and retention time. The column stationary phase along with choice of mobile phase supports hydrophobic interaction in addition to molecular sizing (hydrophobic interaction much milder compared to SMAC). Data analysis was performed using Agilent ChemStation B.04.03.

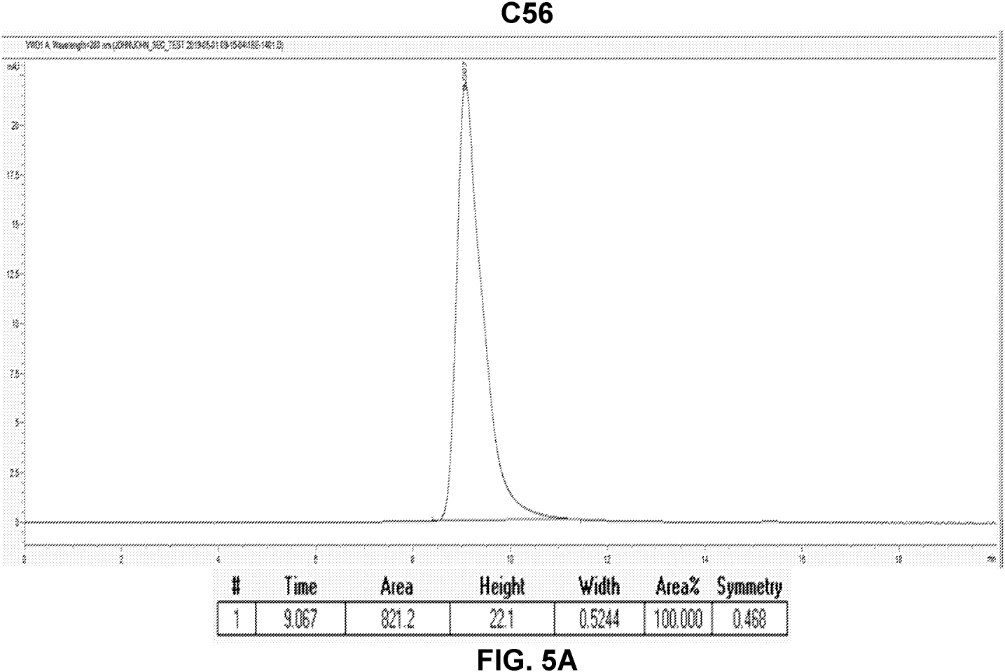

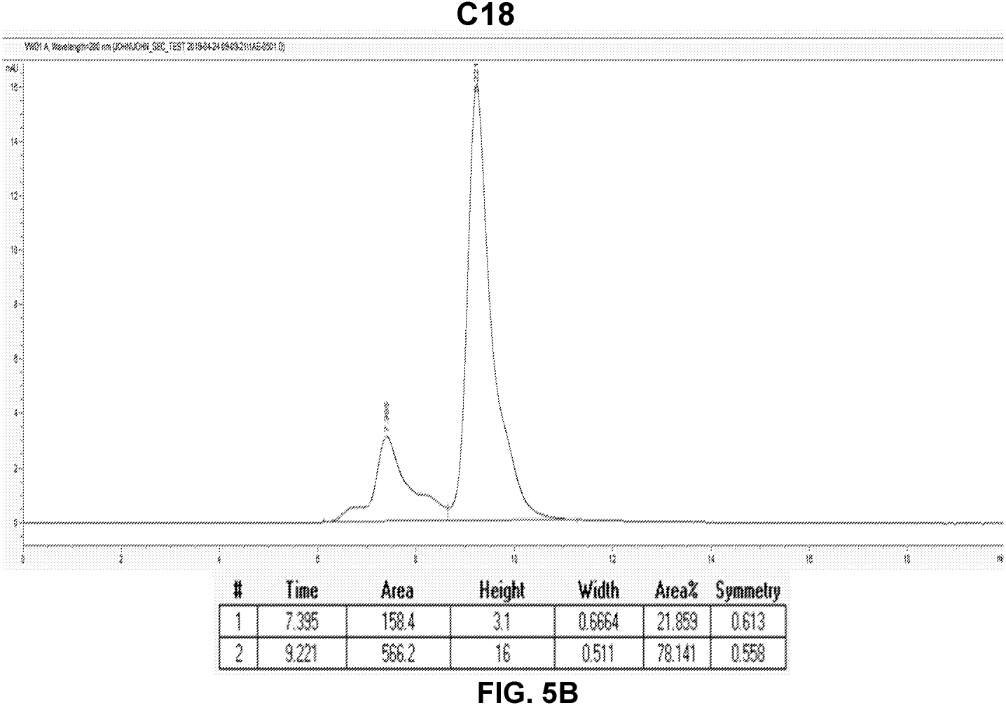

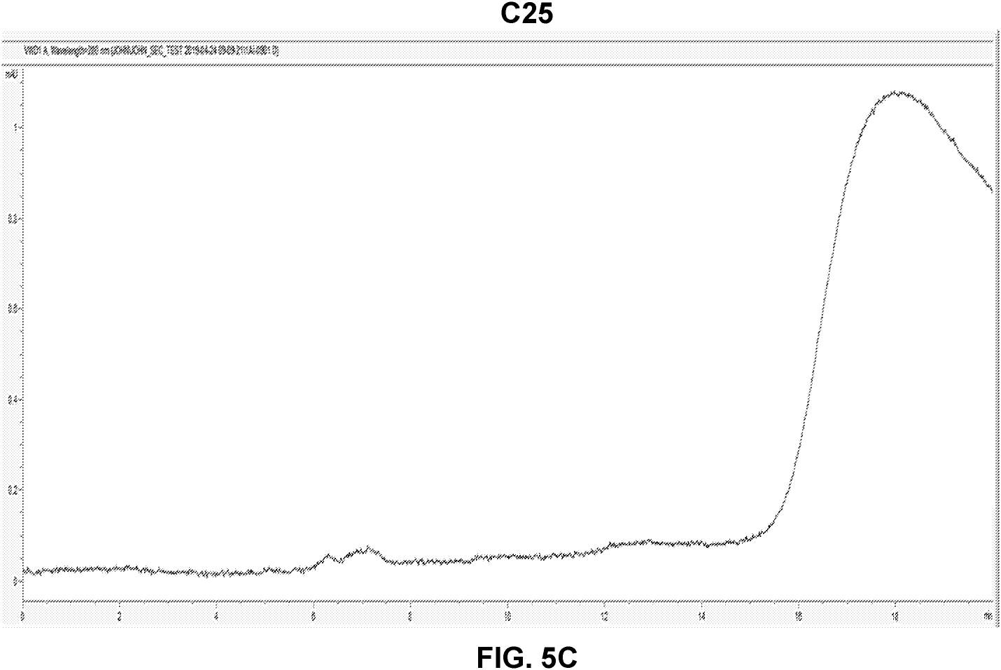

Exemplary SEC results are shown in FIGS. 5A-5C and are summarized in Table 9. Strong developability is indicated by the symbol “+++”, moderate developability is indicated by the symbol “++”, and weak developability is indicated by the symbol “+”. “ND” indicates that developability using SEC was not determined. Results show that 34 antibodies had strong developability based on SEC analysis (C1, C2, C4, C9, C11, C12, C15, C19, C20, C21, C22, C24, C27, C29, C31, C32, C33, C34, C35, C37, C38, C39, C40, C41, C42, C43, C53, C54, C55, C56, C58, C59, C60, and C64), 10 antibodies had moderate developability (C3, C5, C18, C23, C26, C44, C46, C51, C52, and C57), and 1 antibody had weak developability (C25). Results indicate low antibody aggregation and strong or moderate developability based on SEC results for most tested antibodies.

TABLE 9

Developability - Size-Exclusion Chromatography

| | |
|---|---|
| Binder | SEC |
| C1 | +++ |
| C2 | +++ |
| C3 | ++ |
| C4 | +++ |
| C5 | ++ |
| C6 | ND |
| C7 | ND |
| C8 | ND |
| C9 | +++ |
| C10 | ND |
| C11 | +++ |
| C12 | +++ |
| C13 | ND |
| C14 | ND |
| C15 | +++ |
| C16 | ND |
| C17 | ND |
| C18 | ++ |
| C19 | +++ |
| C20 | +++ |
| C21 | +++ |
| C22 | +++ |
| C23 | ++ |
| C24 | +++ |
| C25 | + |
| C26 | ++ |
| C27 | +++ |
| C28 | ND |
| C29 | +++ |
| C30 | ND |
| C31 | +++ |
| C32 | +++ |
| C33 | +++ |
| C34 | +++ |
| C35 | +++ |
| C36 | ND |
| C37 | +++ |
| C38 | +++ |
| C39 | +++ |
| C40 | +++ |
| C41 | +++ |
| C42 | +++ |
| C43 | +++ |
| C44 | ++ |
| C45 | ND |
| C46 | ++ |
| C47 | ND |
| C48 | ND |
| C49 | ND |
| C50 | ND |
| C51 | ++ |
| C52 | ++ |
| C53 | +++ |
| C54 | +++ |
| C55 | +++ |
| C56 | +++ |
| C57 | ++ |
| C58 | +++ |
| C59 | +++ |
| C60 | +++ |
| C61 | ND |
| C62 | ND |
| C63 | ND |
| C64 | +++ |

Hydrophobic interaction chromatography (HIC) analysis was performed using a 4.6 mm ID x 3.5 cm TSKgel Butyl-NPR column (Tosoh Bioscience LLC, PN 14947) on an Agilent 1100 HPLC. Antibodies were normalized to 2 mg/mL concentration in dPBS (pH 7.4) and then diluted with an equal volume of mobile phase buffer B to a final protein concentration of 1 mg/mL. The column was equilibrated with 100% mobile phase Buffer B (2 M ammonium sulfate/20 mM sodium phosphate, pH 7.0) at a flow rate of 1 mL/min. For each sample, 10 µL was loaded and eluted using a gradient from 100% mobile phase buffer B to 100% mobile phase buffer A (20 mM sodium phosphate, pH 7.0) at 1.0 mL/min over 15 min, held at 100% A for 3 min to wash the column, and returned 100% B for 2 min for equilibration. Absorbance was monitored at 280 nm. Sample retention time was calculated and compared to a set of standard controls to identify antibodies with increased retention time (increased hydrophobicity).

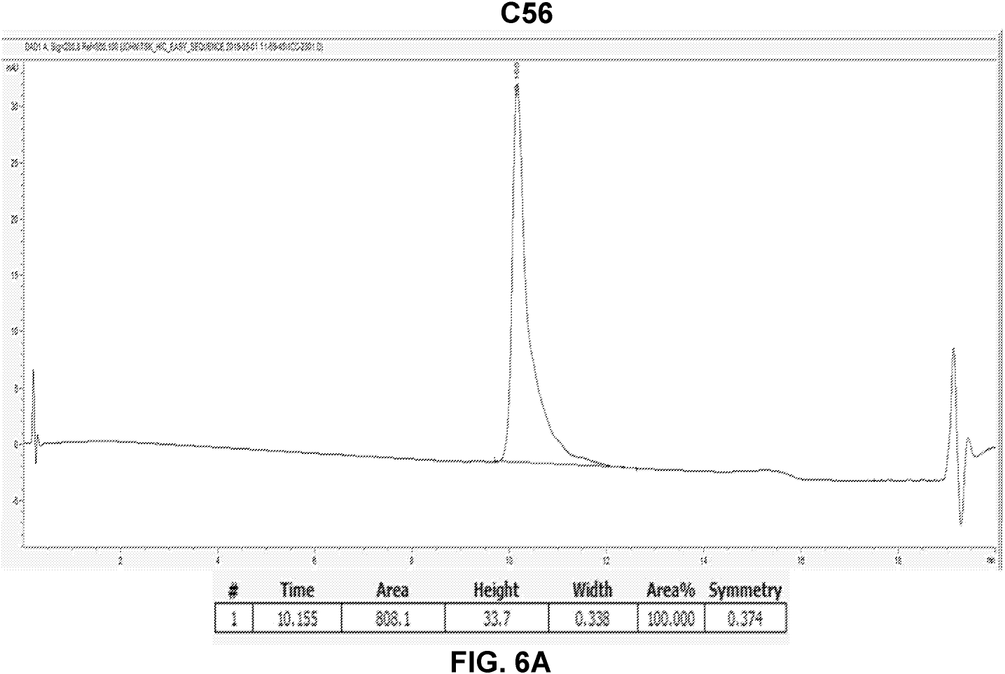

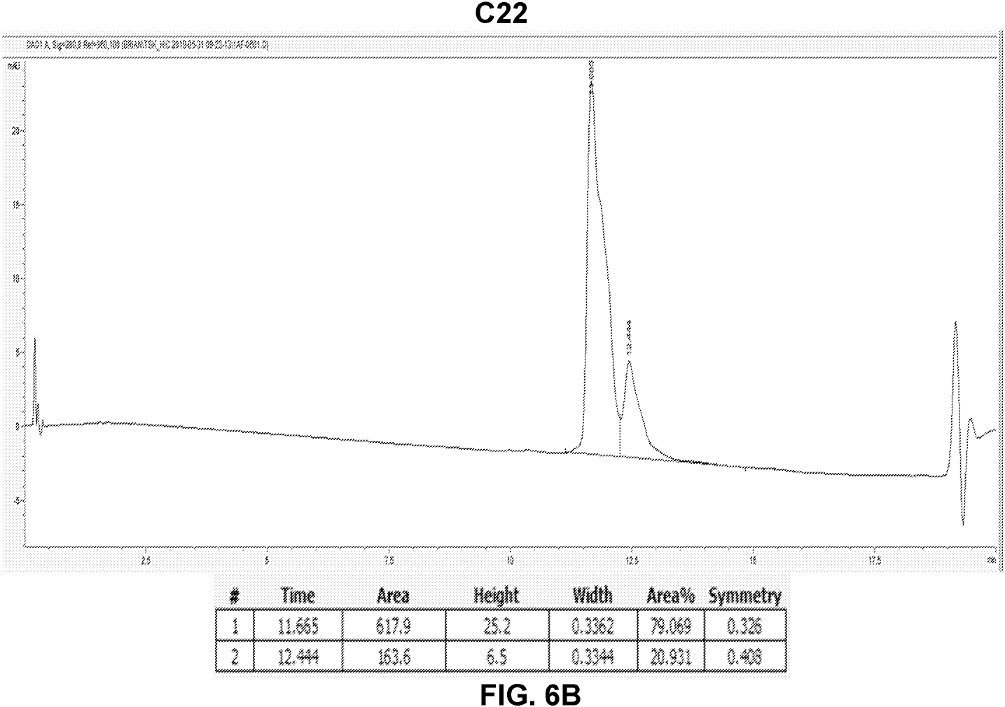

Exemplary HIC results are shown in FIGS. 6A-6B and summarized in Table 10. Strong developability is indicated by the symbol “+++”, moderate developability is indicated by the symbol “++”, and weak developability is indicated by the symbol “+”. “ND” indicates that developability was not determined using HIC. Results show that all tested antibodies had strong or moderate developability based on HIC results. Antibody hydrophobicity can impact antibody aggregation, solubility and viscosity. Results indicate a low propensity for aggregation and precipitation of these antibodies.

TABLE 10

Developability - Hydrophobic Interaction Chromatography

| | |
|---|---|
| Binder | HIC |
| C1 | +++ |
| C2 | +++ |
| C3 | ++ |
| C4 | ++ |
| C5 | ++ |
| C6 | ND |
| C7 | ND |
| C8 | ND |
| C9 | +++ |
| C10 | ND |
| C11 | ++ |
| C12 | ++ |
| C13 | ND |
| C14 | ND |
| C15 | +++ |
| C16 | ND |
| C17 | ND |
| C18 | ++ |
| C19 | +++ |
| C20 | ++ |
| C21 | ++ |
| C22 | ++ |
| C23 | ++ |
| C24 | +++ |
| C25 | +++ |
| C26 | +++ |
| C27 | +++ |
| C28 | ND |
| C29 | ++ |
| C30 | ND |
| C31 | ++ |
| C32 | ++ |
| C33 | ++ |
| C34 | +++ |
| C35 | ++ |
| C36 | ND |
| C37 | +++ |
| C38 | +++ |
| C39 | ++ |
| C40 | +++ |
| C41 | ++ |
| C42 | ++ |
| C43 | +++ |
| C44 | ND |
| C45 | ND |
| C46 | +++ |
| C47 | ND |
| C48 | ND |
| C49 | ND |
| C50 | ND |
| C51 | ++ |
| C52 | ++ |
| C53 | +++ |
| C54 | +++ |
| C55 | ++ |
| C56 | +++ |
| C57 | ++ |
| C58 | +++ |
| C59 | +++ |
| C60 | ++ |
| C61 | ND |
| C62 | ND |
| C63 | ND |
| C64 | +++ |

Standup monolayer adsorption chromatography (SMAC) analysis was performed using a 4.6 mm ID × 300 mm Zenix SEC 300 column (Sepax Technologies, PN 213300P-4630) on an Agilent 1100 HPLC. Antibodies were normalized to 1 mg/mL concentration in dPBS (pH 7.4) and clarified via centrifugation to pellet particulates. The mobile phase buffer was dPBS (pH 7.4, without calcium and magnesium). For each sample, 10 µL was loaded and isocratically eluted at 0.25 mL/min over 32 min. Absorbance was monitored at 280 nm. Sample retention time was calculated and compared to a set of standard controls to identify antibodies with increased retention time (increased propensity to form aggregates).

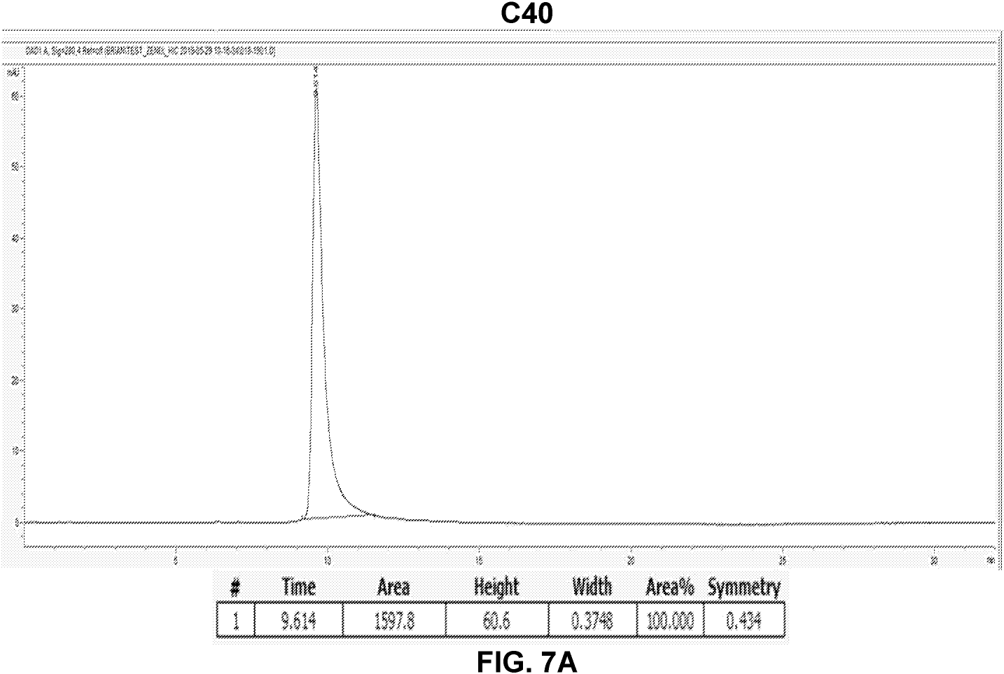

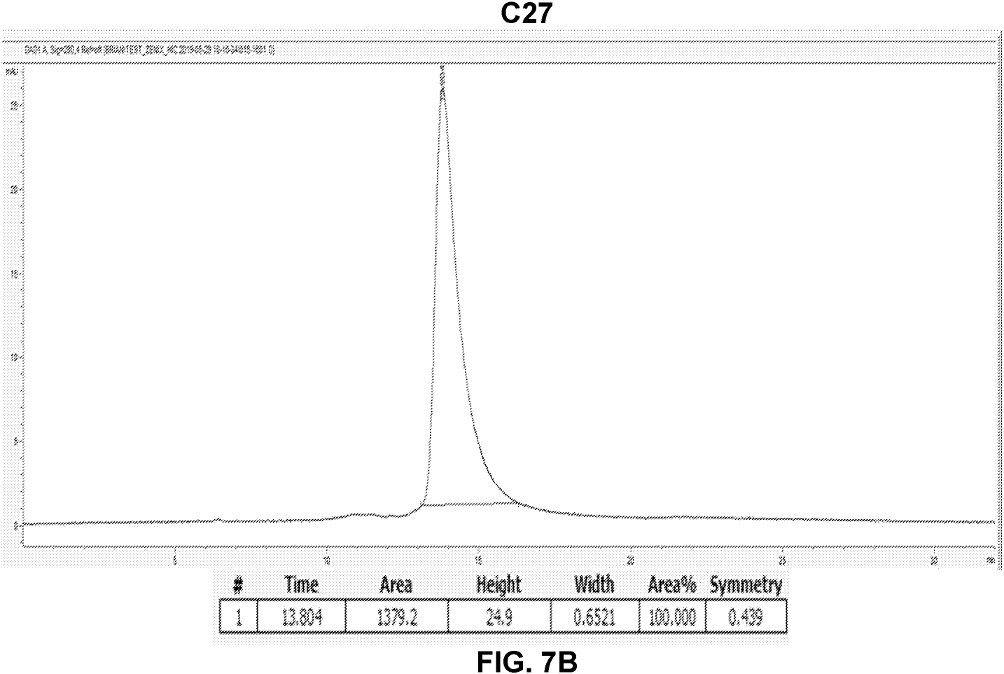

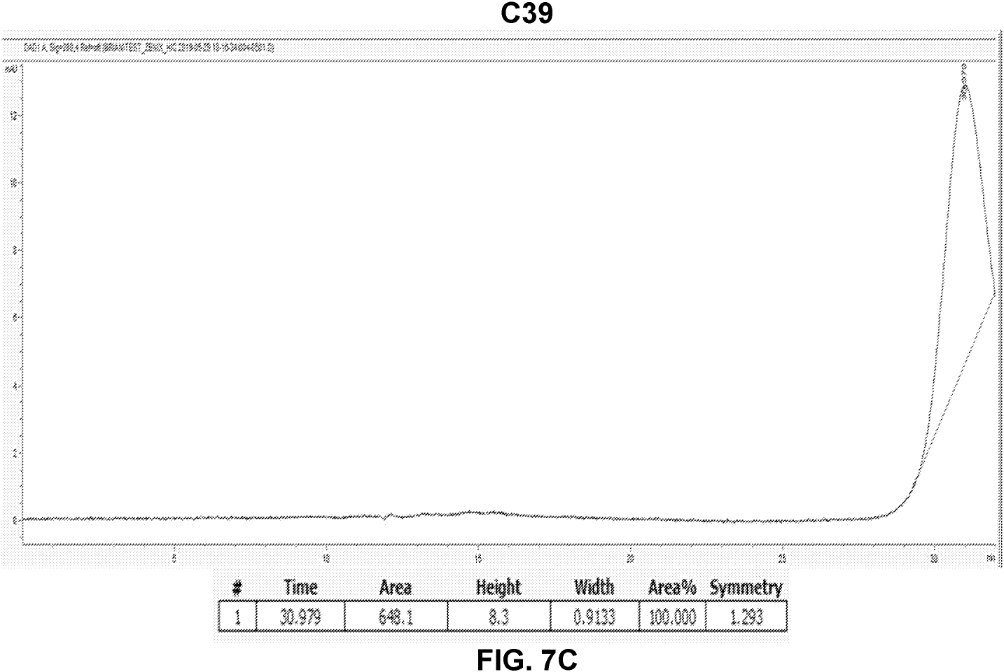

Exemplary SMAC results are shown in FIGS. 7A-7C and summarized in Table 11. Strong developability based on SMAC is indicated by the symbol “+++”, moderate developability is indicated by the symbol “++”, and weak developability is indicated by the symbol “+”. “ND” indicates that developability was not determined using SMAC. Results show that 17 tested antibodies had strong developability based on SMAC (C2, C9, C15, C19, C21, C24, C27, C34, C40, C41, C42, C43, C46, C53, C56, C59, and C60). The determination was based on good retention times, which indicate colloidal stability and low propensity to aggregate.

TABLE 11

Developability - Standup Monolayer Adsorption Chromatography

| | |
|---|---|
| Binder | SMAC |
| C1 | ++ |
| C2 | +++ |
| C3 | + |
| C4 | + |
| C5 | + |
| C6 | ND |
| C7 | ND |
| C8 | ND |
| C9 | +++ |
| C10 | ND |
| C11 | + |
| C12 | + |
| C13 | ND |
| C14 | ND |
| C15 | +++ |
| C16 | ND |
| C17 | ND |
| C18 | ++ |
| C19 | +++ |
| C20 | ++ |
| C21 | +++ |
| C22 | ++ |
| C23 | + |
| C24 | +++ |
| C25 | + |
| C26 | ++ |
| C27 | +++ |
| C28 | ND |
| C29 | + |
| C30 | ND |
| C31 | ++ |
| C32 | ++ |
| C33 | ++ |
| C34 | +++ |
| C35 | ++ |
| C36 | ND |
| C37 | + |
| C38 | ++ |
| C39 | + |
| C40 | +++ |
| C41 | +++ |
| C42 | +++ |
| C43 | +++ |
| C44 | ND |
| C45 | ND |
| C46 | +++ |
| C47 | ND |
| C48 | ND |
| C49 | ND |
| C50 | ND |
| C51 | ++ |
| C52 | ++ |
| C53 | +++ |
| C54 | ++ |
| C55 | ++ |
| C56 | +++ |
| C57 | ++ |
| C58 | +++ |
| C59 | ++ |
| C60 | +++ |
| C61 | ND |
| C62 | ND |
| C63 | ND |
| C64 | ++ |

Based on the combined results of the cell binding assays of Example 3, the cell blocking assays of Example 4, the functional assays of Example 5, and the developability assays of Example 715 antibodies were selected for further analysis: C2, C9, C12, C20, C21, C22, C29, C31, C33, C38, C40, C54, C55, C56, and C59.

The UNcle analytical instrument (Unchained Labs) was employed to assess the biostability of antibodies using multiple assays. Stability analysis of engineered antibody variants was evaluated by measuring the Polydispersity Index (PDI), Hydrodynamic Diameter (Z-ave D), Melting Temperature (Tm), and Aggregation Temperature (Tagg).

Antibodies were assayed at concentrations ranging from to 1-20 mg/mL formulated in Dulbecco’s PBS (pH 7.4, without Ca²⁺/Mg²⁺) and clarified via centrifugation to pellet large particulates while still retaining soluble aggregates. Samples were aliquoted into UNcle’s 9 µL quartz capillary cuvette device (Uni) and sealed. PDI and hydrodynamic diameter were measured by DLS at 15° C. The temperature was ramped from 15° C. to 95° C. at 0.5° C./min during which Tm and Tagg were measured by fluorescence and SLS (266 nm, filter 4; 473 nm, filter 3), respectively. Data were analyzed using UNcle Analysis Software v 3.1 or v 3.2.

Exemplary biostability results are shown in Table 12.

TABLE 12

Biostability Assessment

| Binder | PDI | Z-ave D (nm) | Tm | Tagg 266 | Tagg 473 |
|---|---|---|---|---|---|
| C1 | 0.65 | 33 | 69 | 72 | 66 |
| C2 | 0.093 | 33 | 69 | 78 | 78 |
| C3 | 0.266 | 16 | 70 | 74 | 74 |
| C4 | 0.25 | 17 | 70 | 69 | 68 |
| C5 | 0.451 | 18 | 72 | 77 | 76 |
| C6 | ND | ND | ND | ND | ND |
| C7 | ND | ND | ND | ND | ND |
| C8 | ND | ND | ND | ND | ND |
| C9 | 2.172 | 38 | 73 | 75 | 75 |
| C10 | ND | ND | ND | ND | ND |
| C11 | 0.387 | 41 | 72 | 76 | 74 |
| C12 | 0.388 | 21 | 73 | 77 | 69 |
| C13 | ND | ND | ND | ND | ND |
| C14 | ND | ND | ND | ND | ND |
| C15 | 0.725 | 27 | 72 | 80 | 80 |
| C16 | ND | ND | ND | ND | ND |
| C17 | ND | ND | ND | ND | ND |
| C18 | 0.795 | 42.78 | 71 | 75 | 76 |
| C19 | 0.616 | 28.3 | 73 | 78.71 | 79.12 |
| C20 | 0.618 | 29.97 | 70 | 72 | 73 |
| C21 | 1.431 | 50.71 | 71 | 76 | 77 |
| C22 | 1.061 | 49.76 | 76 | 76 | 75 |
| C23 | 0.325 | 17.9 | 72 | 76.24 | 76.76 |
| C24 | 0.433 | 33.88 | 71 | 78 | 79 |
| C25 | 0.399 | 28.23 | 76 | 74 | 73 |
| C26 | 0.41 | 20.21 | 70 | 72 | 71 |
| C27 | 0.532 | 27.25 | 70.29 | 74.4 | 75.65 |
| C28 | ND | ND | ND | ND | ND |
| C29 | 0.337 | 15.38 | 72 | 74 | 72 |
| C30 | ND | ND | ND | ND | ND |
| C31 | 1.013 | 39.49 | 73 | 78 | 79 |
| C32 | ND | ND | ND | ND | ND |
| C33 | 2.327 | 60.37 | 71 | 73 | 74 |
| C34 | 0.134 | 22.65 | 72.3 | 79.68 | 79.84 |
| C35 | 0.898 | 33.4 | 71.48 | 74.9 | 75.4 |
| C36 | ND | ND | ND | ND | ND |
| C37 | 1.416 | 61.15 | 76 | 75 | 74 |
| C38 | 2.148 | 47.28 | 74 | 79 | 78 |
| C39 | 0.051 | 21.95 | 70 | 75 | 75 |
| C40 | 0.819 | 26.75 | 73 | 79 | 79 |
| C41 | 1.224 | 49.26 | 73 | 80 | 81 |
| C42 | 0.295 | 23.6 | 71 | 79 | 79 |
| C43 | 0.218 | 10.43 | 70 | 75 | 75 |
| C44 | ND | ND | ND | ND | ND |
| C45 | ND | ND | ND | ND | ND |
| C46 | 0.155 | 17.69 | 68 | 72 | 72 |
| C47 | ND | ND | ND | ND | ND |
| C48 | ND | ND | ND | ND | ND |
| C49 | ND | ND | ND | ND | ND |
| C50 | ND | ND | ND | ND | ND |
| C51 | 2.097 | 30.05 | 78 | 76 | 77 |
| C52 | 0.247 | 18.07 | 77 | 76 | 77 |
| C53 | 0.352 | 14.09 | 76 | 78 | 79 |
| C54 | 0.69 | 33.85 | 76 | 76 | 76 |
| C55 | 0.952 | 40.09 | 73 | 79 | 80 |
| C56 | 0.849 | 30.23 | 79 | 77 | 78 |
| C57 | 0.402 | 21.29 | 72 | 79 | 79 |
| C58 | 0.098 | 25.18 | 71 | 78 | 79 |
| C59 | 0.162 | 16.97 | 77 | 79 | 79 |
| C60 | 0.412 | 26.56 | 73 | 79 | 79 |
| C61 | ND | ND | ND | ND | ND |
| C62 | ND | ND | ND | ND | ND |
| C63 | ND | ND | ND | ND | ND |
| C64 | 0.073 | 18.23 | 71 | 77 | 78 |

### Example 8: Competitive Binding Assays and Epitope Binning

Competitive binding assays were employed to determine if antibodies compete for the same or similar binding region of human CD47. Using a competitive immunoassay, if antigen binding of one antibody prevents the binding of the other, then these two antibodies are considered to bind to the same or similar, (e.g., overlapping) epitopes, and are considered to be in the same epitope bin. If binding of an antibody does not interfere with the binding of another antibody, then they are considered to bind to distinct epitopes of CD47, and are in different epitope bins.

An octet-based “in tandem” assay format was used for the cross-competition assays to establish competitive binding data and epitope binning. For these assays, 100 nM biotinylated antigen was immobilized on a streptavidin sensor in 10X kinetic buffer (ForteBio). The association of antibodies to CD47 were monitored by dipping the sensor in consecutive steps into wells containing saturating concentrations of two competing (or non-competing) antibodies. If the saturation with the first antibody did not block the binding (indicated by further increment in the BLI signal) then the antibodies were considered to be binding to distinct or non-overlapping epitopes and belong to different bins.

Results indicate that C40, C56 and C59 bin together.

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. An antibody or fragment thereof that competes for binding to human CD47 with an antibody comprising: (i) a heavy chain variable region having an amino acid sequence of SEQ ID NO:25 and a light chain variable region having an amino acid sequence of SEQ ID NO:26; (ii) a heavy chain variable region having an amino acid sequence of SEQ ID NO:51 and a light chain variable region having an amino acid sequence of SEQ ID NO:52; or (iii) a heavy chain variable region having an amino acid sequence of SEQ ID NO:77 and a light chain variable region having an amino acid sequence of SEQ ID NO:78.

2. An antibody or fragment thereof that binds to CD47, wherein the antibody or fragment thereof comprises:
(a) a heavy chain variable (V_{H}) region comprising:
(1) a V_{H} CDR1 having an amino acid sequence selected from the group consisting of:
(i) SEQ ID NO:1, 27, or 53,
(ii) SEQ ID NO:7, 33, or 59,
(iii) SEQ ID NO:12, 38, or 64,
(iv) SEQ ID NO:13, 39, or 65, and
(v) SEQ ID NO:18, 44, or 70;
(2) a V_{H} CDR2 having an amino acid sequence selected from the group consisting of:
(i) SEQ ID NO:2, 28, or 54,
(ii) SEQ ID NO:8, 34, or 60,
(iii) SEQ ID NO:14, 40, or 66,
(iv) SEQ ID NO:19, 45, or 71, and
(v) SEQ ID NO:24, 50, or 76; and
(3) a V_{H} CDR3 having an amino acid sequence selected from the group consisting of:
(i) SEQ ID NO:3, 29, or 55,
(ii) SEQ ID NO:9, 35, or 61,
(iii) SEQ ID NO: 15, 41, or 67, and
(iv) SEQ ID NO:20, 46, or 72; and
(b) a light chain variable (V_{L}) region comprising:
(1) a V_{L} CDR1 having an amino acid sequence selected from the group consisting of:
(i) SEQ ID NO:4, 30, or 56,
(ii) SEQ ID NO:10, 36, or 62,
(iii) SEQ ID NO:16, 42, or 68, and
(iv) SEQ ID NO:21, 47, or 73;
(2) a V_{L} CDR2 having an amino acid sequence selected from the group consisting of:
(i) SEQ ID NO:5, 31, or 57,
(ii) SEQ ID NO:11, 37, or 63, and
(iii) SEQ ID NO:22, 48, or 74; and
(3) a V_{L} CDR3 having an amino acid sequence selected from the group consisting of:
(i) SEQ ID NO:6, 32, or 58,
(ii) SEQ ID NO:17, 43, or 69, and
(iii) SEQ ID NO:23, 49, or 75.

3. An antibody or fragment thereof that binds to CD47, wherein the antibody or fragment thereof comprises a heavy chain variable (V_{H}) region comprising:
(1) a V_{H} CDR1 having an amino acid sequence selected from the group consisting of:
(i) SEQ ID NO:1, 27, or 53,
(ii) SEQ ID NO:7, 33, or 59,
(iii) SEQ ID NO:12, 38, or 64,
(iv) SEQ ID NO:13, 39, or 65, and
(v) SEQ ID NO:18, 44, or 70;
(2) a V_{H} CDR2 having an amino acid sequence selected from the group consisting of:
(i) SEQ ID NO:2, 28, or 54,
(ii) SEQ ID NO:8, 34, or 60,
(iii) SEQ ID NO:14, 40, or 66,
(iv) SEQ ID NO:19, 45, or 71, and
(v) SEQ ID NO:24, 50, or 76; and
(3) a V_{H} CDR3 having an amino acid sequence selected from the group consisting of:
(i) SEQ ID NO:3, 29, or 55,
(ii) SEQ ID NO:9, 35, or 61,
(iii) SEQ ID NO:15, 41, or 67, and
(iv) SEQ ID NO:20, 46, or 72.

4. An antibody or fragment thereof that binds to CD47, wherein the antibody or fragment thereof comprises a light chain variable (V_{L}) region comprising:
(1) a V_{L} CDR1 having an amino acid sequence selected from the group consisting of:
(i) SEQ ID NO:4, 30, or 56,
(ii) SEQ ID NO:10, 36, or 62,
(iii) SEQ ID NO:16, 42, or 68, and
(iv) SEQ ID NO:21, 47, or 73;
(2) a V_{L} CDR2 having an amino acid sequence selected from the group consisting of:
(i) SEQ ID NO:5, 31, or 57,
(ii) SEQ ID NO:11, 37, or 63, and
(iii) SEQ ID NO:22, 48, or 74; and
(3) a V_{L} CDR3 having an amino acid sequence selected from the group consisting of:
(i) SEQ ID NO:6, 32, or 58,
(ii) SEQ ID NO:17, 43, or 69, and
(iii) SEQ ID NO:23, 49, or 75.

5. An antibody or fragment thereof that binds to CD47 comprising all three heavy chain complementarity determining regions (CDRs) or all three light chain CDRs from:
the antibody designated C40 that comprises a VH sequence that is SEQ ID NO:25 and a VL sequence that is SEQ ID NO:26;
the antibody designated C56 that comprises a VH sequence that is SEQ ID NO:51 and a VL sequence that is SEQ ID NO:52; or
the antibody designated C59 that comprises a VH sequence that is SEQ ID NO:77 and a VL sequence that is SEQ ID NO:78.

6. The antibody or fragment thereof of claim 5, wherein the antibody or fragment thereof comprises all three heavy chain CDRs and all three light chain CDRs from the antibody designated C40.

7. The antibody or fragment thereof of claim 5, wherein the antibody or fragment thereof comprises all three heavy chain CDRs and all three light chain CDRs from the antibody designated C56.

8. The antibody or fragment thereof of claim 5, wherein the antibody or fragment thereof comprises all three heavy chain CDRs and all three light chain CDRs from the antibody designated C59.

9. An antibody or fragment thereof that binds to CD47, wherein the antibody comprises:
(a) a heavy chain variable (V_{H}) region comprising a V_{H} CDR1, a V_{H} CDR2, and a V_{H} CDR3 amino acid sequence depicted in Tables 1-3; or
(b) a light chain variable (V_{L}) region comprising a V_{L} CDR1, a V_{L} CDR2, and a V_{L} CDR3 amino acid sequence depicted in Tables 1-3.

10. The antibody or fragment thereof of claim 9, wherein the antibody comprises:
(a) a heavy chain variable (V_{H}) region comprising a V_{H} CDR1, a V_{H} CDR2, and a V_{H} CDR3 amino acid sequence depicted in Tables 1-3; and
(b) a light chain variable (V_{L}) region comprising a V_{L} CDR1, a V_{L} CDR2, and a V_{L} CDR3 amino acid sequence depicted in Tables 1-3.

11. The antibody or fragment thereof of claim 9, wherein the antibody comprises a heavy chain variable (V_{H}) region comprising a V_{H} CDR1, a V_{H} CDR2, and a V_{H} CDR3 amino acid sequence depicted in Tables 1-3.

12. The antibody or fragment thereof of claim 9, wherein the antibody comprises a light chain variable (V_{L}) region comprising a V_{L} CDR1, a V_{L} CDR2, and a V_{L} CDR3 amino acid sequence depicted in Tables 1-3.

13. The antibody or fragment thereof of claim 9, wherein the antibody comprises:
(a) a heavy chain variable (V_{H}) region comprising:
(1) a V_{H} CDR1 having an amino acid sequence selected from the group consisting of SEQ ID NO: 1, 7, 12, 13, and 18;
(2) a V_{H} CDR2 having an amino acid sequence selected from the group consisting of SEQ ID NO:2, 8, 14, 19 and 24; and
(3) a V_{H} CDR3 having an amino acid sequence selected from the group consisting of SEQ ID NO:3, 9, 15 and 20; and
(b) a light chain variable (V_{L}) region comprising:
(1) a V_{L} CDR1 having an amino acid sequence selected from the group consisting of SEQ ID NO:4, 10, 16 and 21;
(2) a V_{L} CDR2 having an amino acid sequence selected from the group consisting of SEQ ID NO:5, 11, and 22; and
(3) a V_{L} CDR3 having an amino acid sequence selected from the group consisting of SEQ ID NO:6, 17, and 23.

14. The antibody or fragment thereof of claim 13, wherein the antibody comprises:
(a) a heavy chain variable (V_{H}) region comprising:
(1) a V_{H} CDR1 having the amino acid sequence of SEQ ID NO:1;
(2) a V_{H} CDR2 having the amino acid sequence of SEQ ID NO:2; and
(3) a V_{H} CDR3 having the amino acid sequence of SEQ ID NO:3; and
(b) a light chain variable (V_{L}) region comprising:
(1) a V_{L} CDR1 having the amino acid sequence of SEQ ID NO:4;
(2) a V_{L} CDR2 having the amino acid sequence of SEQ ID NO:5; and
(3) a V_{L} CDR3 having the amino acid sequence of SEQ ID NO:6.

15. The antibody or fragment thereof of claim 13, wherein the antibody comprises:
(a) a heavy chain variable (V_{H}) region comprising:
(1) a V_{H} CDR1 having the amino acid sequence of SEQ ID NO:7;
(2) a V_{H} CDR2 having the amino acid sequence of SEQ ID NO:8; and
(3) a V_{H} CDR3 having the amino acid sequence of SEQ ID NO:9; and
(b) a light chain variable (V_{L}) region comprising:
(1) a V_{L} CDR1 having the amino acid sequence of SEQ ID NO:10;
(2) a V_{L} CDR2 having the amino acid sequence of SEQ ID NO:11; and
(3) a V_{L} CDR3 having the amino acid sequence of SEQ ID NO:6.

16. The antibody or fragment thereof of claim 13, wherein the antibody comprises:
(a) a heavy chain variable (V_{H}) region comprising:
(1) a V_{H} CDR1 having the amino acid sequence of SEQ ID NO:12;
(2) a V_{H} CDR2 having the amino acid sequence of SEQ ID NO:2; and
(3) a V_{H} CDR3 having the amino acid sequence of SEQ ID NO:3; and
(b) a light chain variable (V_{L}) region comprising:
(1) a V_{L} CDR1 having the amino acid sequence of SEQ ID NO:4;
(2) a V_{L} CDR2 having the amino acid sequence of SEQ ID NO:5; and
(3) a V_{L} CDR3 having the amino acid sequence of SEQ ID NO:6.

17. The antibody or fragment thereof of claim 13, wherein the antibody comprises:
(a) a heavy chain variable (V_{H}) region comprising:
(1) a V_{H} CDR1 having the amino acid sequence of SEQ ID NO:13;
(2) a V_{H} CDR2 having the amino acid sequence of SEQ ID NO:14; and
(3) a V_{H} CDR3 having the amino acid sequence of SEQ ID NO:15; and
(b) a light chain variable (V_{L}) region comprising:
(1) a V_{L} CDR1 having the amino acid sequence of SEQ ID NO:16;
(2) a V_{L} CDR2 having the amino acid sequence of SEQ ID NO:11; and
(3) a V_{L} CDR3 having the amino acid sequence of SEQ ID NO:17.

18. The antibody or fragment thereof of claim 13, wherein the antibody comprises:
(a) a heavy chain variable (V_{H}) region comprising:
(1) a V_{H} CDR1 having the amino acid sequence of SEQ ID NO:18;
(2) a V_{H} CDR2 having the amino acid sequence of SEQ ID NO:19; and
(3) a V_{H} CDR3 having the amino acid sequence of SEQ ID NO:20; and
(b) a light chain variable (V_{L}) region comprising:
(1) a V_{L} CDR1 having the amino acid sequence of SEQ ID NO:21;
(2) a V_{L} CDR2 having the amino acid sequence of SEQ ID NO:22; and
(3) a V_{L} CDR3 having the amino acid sequence of SEQ ID NO:23.

19. The antibody or fragment thereof of claim 13, wherein the antibody comprises:
(a) a heavy chain variable (V_{H}) region comprising:
(1) a V_{H} CDR1 having the amino acid sequence of SEQ ID NO:1;
(2) a V_{H} CDR2 having the amino acid sequence of SEQ ID NO:24; and
(3) a V_{H} CDR3 having the amino acid sequence of SEQ ID NO:3; and
(b) a light chain variable (V_{L}) region comprising:
(1) a V_{L} CDR1 having the amino acid sequence of SEQ ID NO:4;
(2) a V_{L} CDR2 having the amino acid sequence of SEQ ID NO:5; and
(3) a V_{L} CDR3 having the amino acid sequence of SEQ ID NO:6.

20. The antibody or fragment thereof of claim 9, wherein the antibody comprises:
(a) a heavy chain variable (V_{H}) region comprising:
(1) a V_{H} CDR1 having an amino acid sequence selected from the group consisting of SEQ ID NO:27, 33, 38, 39 and 44;
(2) a V_{H} CDR2 having an amino acid sequence selected from the group consisting of SEQ ID NO:28, 34, 40, 45 and 50; and
(3) a V_{H} CDR3 having an amino acid sequence selected from the group consisting of SEQ ID NO:29, 35, 41 and 46; and
(b) a light chain variable (V_{L}) region comprising:
(1) a V_{L} CDR1 having an amino acid sequence selected from the group consisting of SEQ ID NO:30, 36, 42, and 47;
(2) a V_{L} CDR2 having an amino acid sequence selected from the group consisting of SEQ ID NO:31, 37 and 48 and
(3) a V_{L} CDR3 having an amino acid sequence selected from the group consisting of SEQ ID NO:32, 43 and 49.

21. The antibody or fragment thereof of claim 20, wherein the antibody comprises:
(a) a heavy chain variable (V_{H}) region comprising:
(1) a V_{H} CDR1 having the amino acid sequence of SEQ ID NO:27;
(2) a V_{H} CDR2 having the amino acid sequence of SEQ ID NO:28; and
(3) a V_{H} CDR3 having the amino acid sequence of SEQ ID NO:29; and
(b) a light chain variable (V_{L}) region comprising:
(1) a V_{L} CDR1 having the amino acid sequence of SEQ ID NO:30;
(2) a V_{L} CDR2 having the amino acid sequence of SEQ ID NO:31; and
(3) a V_{L} CDR3 having the amino acid sequence of SEQ ID NO:32.

22. The antibody or fragment thereof of claim 20, wherein the antibody comprises:
(a) a heavy chain variable (V_{H}) region comprising:
(1) a V_{H} CDR1 having the amino acid sequence of SEQ ID NO:33;
(2) a V_{H} CDR2 having the amino acid sequence of SEQ ID NO:34; and
(3) a V_{H} CDR3 having the amino acid sequence of SEQ ID NO:35; and
(b) a light chain variable (V_{L}) region comprising:
(1) a V_{L} CDR1 having the amino acid sequence of SEQ ID NO:36;
(2) a V_{L} CDR2 having the amino acid sequence of SEQ ID NO:37; and
(3) a V_{L} CDR3 having the amino acid sequence of SEQ ID NO:32.

23. The antibody or fragment thereof of claim 20, wherein the antibody comprises:
(a) a heavy chain variable (V_{H}) region comprising:
(1) a V_{H} CDR1 having the amino acid sequence of SEQ ID NO:38;
(2) a V_{H} CDR2 having the amino acid sequence of SEQ ID NO:28; and
(3) a V_{H} CDR3 having the amino acid sequence of SEQ ID NO:29; and
(b) a light chain variable (V_{L}) region comprising:
(1) a V_{L} CDR1 having the amino acid sequence of SEQ ID NO:30;
(2) a V_{L} CDR2 having the amino acid sequence of SEQ ID NO:31; and
(3) a V_{L} CDR3 having the amino acid sequence of SEQ ID NO:32.

24. The antibody or fragment thereof of claim 20, wherein the antibody comprises:
(a) a heavy chain variable (V_{H}) region comprising:
(1) a V_{H} CDR1 having the amino acid sequence of SEQ ID NO:39;
(2) a V_{H} CDR2 having the amino acid sequence of SEQ ID NO:40; and
(3) a V_{H} CDR3 having the amino acid sequence of SEQ ID NO:41; and
(b) a light chain variable (V_{L}) region comprising:
(1) a V_{L} CDR1 having the amino acid sequence of SEQ ID NO:42;
(2) a V_{L} CDR2 having the amino acid sequence of SEQ ID NO:37; and
(3) a V_{L} CDR3 having the amino acid sequence of SEQ ID NO:43.

25. The antibody or fragment thereof of claim 20, wherein the antibody comprises:
(a) a heavy chain variable (V_{H}) region comprising:
(1) a V_{H} CDR1 having the amino acid sequence of SEQ ID NO:44;
(2) a V_{H} CDR2 having the amino acid sequence of SEQ ID NO:45; and
(3) a V_{H} CDR3 having the amino acid sequence of SEQ ID NO:46; and
(b) a light chain variable (V_{L}) region comprising:
(1) a V_{L} CDR1 having the amino acid sequence of SEQ ID NO:47;
(2) a V_{L} CDR2 having the amino acid sequence of SEQ ID NO:48; and
(3) a V_{L} CDR3 having the amino acid sequence of SEQ ID NO:49.

26. The antibody or fragment thereof of claim 20, wherein the antibody comprises:
(a) a heavy chain variable (V_{H}) region comprising:
(1) a V_{H} CDR1 having the amino acid sequence of SEQ ID NO:27;
(2) a V_{H} CDR2 having the amino acid sequence of SEQ ID NO:50; and
(3) a V_{H} CDR3 having the amino acid sequence of SEQ ID NO:29; and
(b) a light chain variable (V_{L}) region comprising:
(1) a V_{L} CDR1 having the amino acid sequence of SEQ ID NO:30;
(2) a V_{L} CDR2 having the amino acid sequence of SEQ ID NO:31; and
(3) a V_{L} CDR3 having the amino acid sequence of SEQ ID NO:32.

27. The antibody or fragment thereof of claim 9, wherein the antibody comprises:
(a) a heavy chain variable (V_{H}) region comprising:
(1) a V_{H} CDR1 having an amino acid sequence selected from the group consisting of SEQ ID NO:53, 59, 64, 65, and 70;
(2) a V_{H} CDR2 having an amino acid sequence selected from the group consisting of SEQ ID NO:54, 60, 66, 71, and 76; and
(3) a V_{H} CDR3 having an amino acid sequence selected from the group consisting of SEQ ID NO:55, 61, 67, and 72; and
(b) a light chain variable (V_{L}) region comprising:
(1) a V_{L} CDR1 having an amino acid sequence selected from the group consisting of SEQ ID NO:56, 62, 68, and 73;
(2) a V_{L} CDR2 having an amino acid sequence selected from the group consisting of SEQ ID NO:57, 63 and 74; and
(3) a V_{L} CDR3 having an amino acid sequence selected from the group consisting of SEQ ID NO:58, 69, and 75.

28. The antibody or fragment thereof of claim 27, wherein the antibody comprises:
(a) a heavy chain variable (V_{H}) region comprising:
(1) a V_{H} CDR1 having the amino acid sequence of SEQ ID NO:53;
(2) a V_{H} CDR2 having the amino acid sequence of SEQ ID NO:54; and
(3) a V_{H} CDR3 having the amino acid sequence of SEQ ID NO:55; and
(b) a light chain variable (V_{L}) region comprising:
(1) a V_{L} CDR1 having the amino acid sequence of SEQ ID NO:56;
(2) a V_{L} CDR2 having the amino acid sequence of SEQ ID NO:57; and
(3) a V_{L} CDR3 having the amino acid sequence of SEQ ID NO:58.

29. The antibody or fragment thereof of claim 27, wherein the antibody comprises:
(a) a heavy chain variable (V_{H}) region comprising:
(1) a V_{H} CDR1 having the amino acid sequence of SEQ ID NO:59;
(2) a V_{H} CDR2 having the amino acid sequence of SEQ ID NO:60; and
(3) a V_{H} CDR3 having the amino acid sequence of SEQ ID NO:61; and
(b) a light chain variable (V_{L}) region comprising:
(1) a V_{L} CDR1 having the amino acid sequence of SEQ ID NO:62;
(2) a V_{L} CDR2 having the amino acid sequence of SEQ ID NO:63; and
(3) a V_{L} CDR3 having the amino acid sequence of SEQ ID NO:58.

30. The antibody or fragment thereof of claim 27, wherein the antibody comprises:
(a) a heavy chain variable (V_{H}) region comprising:
(1) a V_{H} CDR1 having the amino acid sequence of SEQ ID NO:64;
(2) a V_{H} CDR2 having the amino acid sequence of SEQ ID NO:54; and
(3) a V_{H} CDR3 having the amino acid sequence of SEQ ID NO:55; and
(b) a light chain variable (V_{L}) region comprising:
(1) a V_{L} CDR1 having the amino acid sequence of SEQ ID NO:56;
(2) a V_{L} CDR2 having the amino acid sequence of SEQ ID NO:57; and
(3) a V_{L} CDR3 having the amino acid sequence of SEQ ID NO:58.

31. The antibody or fragment thereof of claim 27, wherein the antibody comprises:
(a) a heavy chain variable (V_{H}) region comprising:
(1) a V_{H} CDR1 having the amino acid sequence of SEQ ID NO:65;
(2) a V_{H} CDR2 having the amino acid sequence of SEQ ID NO:66; and
(3) a V_{H} CDR3 having the amino acid sequence of SEQ ID NO:67; and
(b) a light chain variable (V_{L}) region comprising:
(1) a V_{L} CDR1 having the amino acid sequence of SEQ ID NO:68;
(2) a V_{L} CDR2 having the amino acid sequence of SEQ ID NO:63; and
(3) a V_{L} CDR3 having the amino acid sequence of SEQ ID NO:69.

32. The antibody or fragment thereof of claim 27, wherein the antibody comprises:
(a) a heavy chain variable (V_{H}) region comprising:
(1) a V_{H} CDR1 having the amino acid sequence of SEQ ID NO:70;
(2) a V_{H} CDR2 having the amino acid sequence of SEQ ID NO:71; and
(3) a V_{H} CDR3 having the amino acid sequence of SEQ ID NO:72; and
(b) a light chain variable (V_{L}) region comprising:
(1) a V_{L} CDR1 having the amino acid sequence of SEQ ID NO:73;
(2) a V_{L} CDR2 having the amino acid sequence of SEQ ID NO:74; and
(3) a V_{L} CDR3 having the amino acid sequence of SEQ ID NO:75.

33. The antibody or fragment thereof of claim 27, wherein the antibody comprises:
(a) a heavy chain variable (V_{H}) region comprising:
(1) a V_{H} CDR1 having the amino acid sequence of SEQ ID NO:53;
(2) a V_{H} CDR2 having the amino acid sequence of SEQ ID NO:76; and
(3) a V_{H} CDR3 having the amino acid sequence of SEQ ID NO:55; and
(b) a light chain variable (V_{L}) region comprising:
(1) a V_{L} CDR1 having the amino acid sequence of SEQ ID NO:56;
(2) a V_{L} CDR2 having the amino acid sequence of SEQ ID NO:57; and
(3) a V_{L} CDR3 having the amino acid sequence of SEQ ID NO:58.

34. The antibody or fragment thereof of any one of claims 9-33, wherein the V_{H} region or V_{L} region further comprises human framework sequences.

35. The antibody or fragment thereof of claim 37, wherein the V_{H} region and V_{L} region further comprises human framework sequences.

36. The antibody or fragment thereof of any one of claims 9-33, wherein the V_{H} region or V_{L} region further comprises a framework 1 (FR1), a framework 2 (FR2), a framework 3 (FR3) and/or a framework 4 (FR4) sequence.

37. The antibody or fragment thereof of claim 36, wherein the V_{H} region and V_{L} region further comprises a framework 1 (FR1), a framework 2 (FR2), a framework 3 (FR3) and a framework 4 (FR4) sequence.

38. The antibody or fragment thereof of any one of claims 1-37, wherein the antibody is a monoclonal antibody.

39. The antibody or fragment thereof of claim 38, wherein the monoclonal antibody is a humanized, human or chimeric antibody.

40. The antibody or fragment thereof of any one of claims 1-39, which is a Fab, Fab′, F(ab′)₂, Fv, scFv, (scFv)₂, single chain antibody molecule, dual variable region antibody, single variable region antibody, linear antibody, V region, or a multispecific antibody formed from antibody fragments.

41. The antibody or fragment thereof of any one of claims 1-40, which is conjugated or recombinantly fused to a diagnostic agent, detectable agent or therapeutic agent.

42. The antibody or fragment thereof of claim 41, wherein the therapeutic agent is a chemotherapeutic agent, cytotoxin, or drug.

43. A binding agent that binds to essentially the same epitope as an antibody or fragment thereof of any one of claims 1-42.

44. The binding agent of claim 43, which is an antibody or fragment thereof.

45. The binding agent of claim 43, which comprises a non-antibody protein scaffold.

46. The binding agent of claim 45, wherein the non-antibody protein scaffold comprises a fibronectin scaffold, an anticalin, an adnectin, an affibody, a DARPin, a fynomer, an affitin, an affilin, an avimer, a cysteine-rich knottin peptide, or an engineered Kunitz-type inhibitor.

47. A binding agent that competes for binding to human CD47 with an antibody or fragment thereof of any one of claims 1-42.

48. The binding agent of claim 47, wherein the binding agent is an antibody or fragment thereof.

49. One or more vectors comprising one or more polynucleotides encoding the antibody or fragment thereof of any one of claims 1-42.

50. A pharmaceutical composition that comprises the antibody or fragment thereof of any one of claims 1-39, and a pharmaceutically acceptable carrier.

51. A method for treating a cancer or a tumor in a subject comprising administering to the subject the antibody or fragment thereof of any one of claims 1-42 or the pharmaceutical composition of claim 50.

52. A method for alleviating one or more symptoms associated with a cancer or a tumor in a subject comprising administering to the subject the antibody or fragment thereof of any one of claims 1-42 or the pharmaceutical composition of claim 50.

53. A method for decreasing tumor size in a subject with a tumor comprising administering to the subject the antibody or fragment thereof of any one of claims 1-42 or the pharmaceutical composition of claim 50.

54. A method for enhancing tumor cell removal in a subject with a tumor comprising administering to the subject the antibody or fragment thereof of any one of claims 1-42 or the pharmaceutical composition of claim 50.

55. A method for treating a phagocytic cell dysfunctional disease, disorder or condition in a subject comprising administering to the subject the antibody or fragment thereof of any one of claims 1-42 or the pharmaceutical composition of claim 50.

56. A method for increasing immune cell phagocytosis in a subject comprising administering to the subject the antibody or fragment thereof of any one of claims 1-42 or the pharmaceutical composition of claim 50.

57. The method of claim 56, wherein the immune cell is a macrophage, a neutrophil, a dendritic cell, or a B lymphocyte.

58. The method of claim 56 or 57, wherein the subject is diagnosed with a cancer or a tumor.

59. The method of any one of claims 51-58, wherein the subject is administered one or more therapeutic agents in combination with the antibody or fragment thereof or the pharmaceutical composition.


SEQUENCE LISTING (101 sequences)

SEQ ID NO: 1 - protein, 10 aa, Artificial Sequence
  Antibody C40 VH CDR1 (Exemplary, AbM)
GFTFSYYYIH

SEQ ID NO: 2 - protein, 17 aa, Artificial Sequence
  Antibody C40 VH CDR2 (Exemplary, Kabat)
WIDPYGHSTTYADSVKG

SEQ ID NO: 3 - protein, 8 aa, Artificial Sequence
  Antibody C40 VH CDR3 (Exemplary, Kabat, AbM)
GGRGAMDY

SEQ ID NO: 4 - protein, 11 aa, Artificial Sequence
  Antibody C40 VL CDR1 (Exemplary, Kabat, AbM)
RASQSVSSAVA

SEQ ID NO: 5 - protein, 7 aa, Artificial Sequence
  Antibody C40 VL CDR2 (Exemplary, Kabat, AbM)
SASSLYS

SEQ ID NO: 6 - protein, 9 aa, Artificial Sequence
  Antibody C40 VL CDR3 (Exemplary, IMGT, Kabat, AbM)
QQRYSSLLT

SEQ ID NO: 7 - protein, 8 aa, Artificial Sequence
  Antibody C40 VH CDR1 (IMGT)
GFTFSYYY

SEQ ID NO: 8 - protein, 8 aa, Artificial Sequence
  Antibody C40 VH CDR2 (IMGT)
IDPYGHST

SEQ ID NO: 9 - protein, 10 aa, Artificial Sequence
  Antibody C40 VH CDR3 (IMGT)
ARGGRGAMDY

SEQ ID NO: 10 - protein, 6 aa, Artificial Sequence
  Antibody C40 VL CDR1 (IMGT)
QSVSSA

SEQ ID NO: 11 - protein, 3 aa, Artificial Sequence
  Antibody C40 VL CDR2 (IMGT, Chothia)
SAS

SEQ ID NO: 12 - protein, 5 aa, Artificial Sequence
  Antibody C40 VH CDR1 (Kabat)
YYYIH

SEQ ID NO: 13 - protein, 7 aa, Artificial Sequence
  Antibody C40 VH CDR1 (Chothia)
GFTFSYY

SEQ ID NO: 14 - protein, 4 aa, Artificial Sequence
  Antibody C40 VH CDR2 (Chothia)
PYGH

SEQ ID NO: 15 - protein, 6 aa, Artificial Sequence
  Antibody C40 VH CDR3 (Chothia)
GRGAMD

SEQ ID NO: 16 - protein, 7 aa, Artificial Sequence
  Antibody C40 VL CDR1 (Chothia)
SQSVSSA

SEQ ID NO: 17 - protein, 6 aa, Artificial Sequence
  Antibody C40 VL CDR3 (Chothia)
RYSSLL

SEQ ID NO: 18 - protein, 6 aa, Artificial Sequence
  Antibody C40 VH CDR1 (Contact)
SYYYIH

SEQ ID NO: 19 - protein, 13 aa, Artificial Sequence
  Antibody C40 VH CDR2 (Contact)
WVAWIDPYGHSTT

SEQ ID NO: 20 - protein, 9 aa, Artificial Sequence
  Antibody C40 VH CDR3 (Contact)
ARGGRGAMD

SEQ ID NO: 21 - protein, 7 aa, Artificial Sequence
  Antibody C40 VL CDR1 (Contact)
SSAVAWY

SEQ ID NO: 22 - protein, 10 aa, Artificial Sequence
  Antibody C40 VL CDR2 (Contact)
LLIYSASSLY

SEQ ID NO: 23 - protein, 8 aa, Artificial Sequence
  Antibody C40 VL CDR3 (Contact)
QQRYSSLL

SEQ ID NO: 24 - protein, 10 aa, Artificial Sequence
  Antibody C40 VH CDR2 (AbM)
WIDPYGHSTT

SEQ ID NO: 25 - protein, 114 aa, Artificial Sequence
  Antibody C40 VH Sequence
EVQLVESGGGLVQPGGSLRLSCAASGFTFSYYYIHWVRQAPGKGLEWVAWIDPYGHSTTYADSVKGRFTISADTSKNTAYLQMNSLRAEDTAVYYCARGGRGAMDYWGQGTLVT

SEQ ID NO: 26 - protein, 107 aa, Artificial Sequence
  Antibody C40 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQRYSSLLTFGQGTKVEIK

SEQ ID NO: 27 - protein, 10 aa, Artificial Sequence
  Antibody C56 VH CDR1 (Exemplary, AbM)
GFTFTYYYIH

SEQ ID NO: 28 - protein, 17 aa, Artificial Sequence
  Antibody C56 VH CDR2 (Exemplary, Kabat)
FIDPYSGSTEYADSVKG

SEQ ID NO: 29 - protein, 8 aa, Artificial Sequence
  Antibody C56 VH CDR3 (Exemplary, Kabat, AbM)
GGLYALDY

SEQ ID NO: 30 - protein, 11 aa, Artificial Sequence
  Antibody C56 VL CDR1 (Exemplary, Kabat, AbM)
RASQSVSSAVA

SEQ ID NO: 31 - protein, 7 aa, Artificial Sequence
  Antibody C56 VL CDR2 (Exemplary, Kabat, AbM)
SASSLYS

SEQ ID NO: 32 - protein, 9 aa, Artificial Sequence
  Antibody C56 VL CDR3 (Exemplary, IMGT, Kabat, AbM)
QQGRSDLRT

SEQ ID NO: 33 - protein, 8 aa, Artificial Sequence
  Antibody C56 VH CDR1 (IMGT)
GFTFTYYY

SEQ ID NO: 34 - protein, 8 aa, Artificial Sequence
  Antibody C56 VH CDR2 (IMGT)
IDPYSGST

SEQ ID NO: 35 - protein, 10 aa, Artificial Sequence
  Antibody C56 VH CDR3 (IMGT)
ARGGLYALDY

SEQ ID NO: 36 - protein, 6 aa, Artificial Sequence
  Antibody C56 VL CDR1 (IMGT)
QSVSSA

SEQ ID NO: 37 - protein, 3 aa, Artificial Sequence
  Antibody C56 VL CDR2 (IMGT, Chothia)
SAS

SEQ ID NO: 38 - protein, 5 aa, Artificial Sequence
  Antibody C56 VH CDR1 (Kabat)
YYYIH

SEQ ID NO: 39 - protein, 7 aa, Artificial Sequence
  Antibody C56 VH CDR1 (Chothia)
GFTFTYY

SEQ ID NO: 40 - protein, 4 aa, Artificial Sequence
  Antibody C56 VH CDR2 (Chothia)
PYSG

SEQ ID NO: 41 - protein, 6 aa, Artificial Sequence
  Antibody C56 VH CDR3 (Chothia)
GLYALD

SEQ ID NO: 42 - protein, 7 aa, Artificial Sequence
  Antibody C56 VL CDR1 (Chothia)
SQSVSSA

SEQ ID NO: 43 - protein, 6 aa, Artificial Sequence
  Antibody C56 VL CDR3 (Chothia)
GRSDLR

SEQ ID NO: 44 - protein, 6 aa, Artificial Sequence
  Antibody C56 VH CDR1 (Contact)
TYYYIH

SEQ ID NO: 45 - protein, 13 aa, Artificial Sequence
  Antibody C56 VH CDR2 (Contact)
WVAFIDPYSGSTE

SEQ ID NO: 46 - protein, 9 aa, Artificial Sequence
  Antibody C56 VH CDR3 (Contact)
ARGGLYALD

SEQ ID NO: 47 - protein, 7 aa, Artificial Sequence
  Antibody C56 VL CDR1 (Contact)
SSAVAWY

SEQ ID NO: 48 - protein, 10 aa, Artificial Sequence
  Antibody C56 VL CDR2 (Contact)
LLIYSASSLY

SEQ ID NO: 49 - protein, 8 aa, Artificial Sequence
  Antibody C56 VL CDR3 (Contact)
QQGRSDLR

SEQ ID NO: 50 - protein, 10 aa, Artificial Sequence
  Antibody C56 VH CDR2 (AbM)
FIDPYSGSTE

SEQ ID NO: 51 - protein, 114 aa, Artificial Sequence
  Antibody C56 VH Sequence
EVQLVESGGGLVQPGGSLRLSCAASGFTFTYYYIHWVRQAPGKGLEWVAFIDPYSGSTEYADSVKGRFTISADTSKNTAYLQMNSLRAEDTAVYYCARGGLYALDYWGQGTLVT

SEQ ID NO: 52 - protein, 107 aa, Artificial Sequence
  Antibody C56 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQGRSDLRTFGQGTKVEIK

SEQ ID NO: 53 - protein, 10 aa, Artificial Sequence
  Antibody C59 VH CDR1 (Exemplary, AbM)
GFTFTSYYIH

SEQ ID NO: 54 - protein, 17 aa, Artificial Sequence
  Antibody C59 VH CDR2 (Exemplary, Kabat)
YIDSKHGTTQYADSVKG

SEQ ID NO: 55 - protein, 8 aa, Artificial Sequence
  Antibody C59 VH CDR3 (Exemplary, Kabat, AbM)
GGRSAMDY

SEQ ID NO: 56 - protein, 11 aa, Artificial Sequence
  Antibody C59 VL CDR1 (Exemplary, Kabat, AbM)
RASQSVSSAVA

SEQ ID NO: 57 - protein, 7 aa, Artificial Sequence
  Antibody C59 VL CDR2 (Exemplary, Kabat, AbM)
SASSLYS

SEQ ID NO: 58 - protein, 9 aa, Artificial Sequence
  Antibody C59 VL CDR3 (Exemplary, IMGT, Kabat, AbM)
QQRTTSLLT

SEQ ID NO: 59 - protein, 8 aa, Artificial Sequence
  Antibody C59 VH CDR1 (IMGT)
GFTFTSYY

SEQ ID NO: 60 - protein, 8 aa, Artificial Sequence
  Antibody C59 VH CDR2 (IMGT)
IDSKHGTT

SEQ ID NO: 61 - protein, 10 aa, Artificial Sequence
  Antibody C59 VH CDR3 (IMGT)
ARGGRSAMDY

SEQ ID NO: 62 - protein, 6 aa, Artificial Sequence
  Antibody C59 VL CDR1 (IMGT)
QSVSSA

SEQ ID NO: 63 - protein, 3 aa, Artificial Sequence
  Antibody C59 VL CDR2 (IMGT, Chothia)
SAS

SEQ ID NO: 64 - protein, 5 aa, Artificial Sequence
  Antibody C59 VH CDR1 (Kabat)
SYYIH

SEQ ID NO: 65 - protein, 7 aa, Artificial Sequence
  Antibody C59 VH CDR1 (Chothia)
GFTFTSY

SEQ ID NO: 66 - protein, 4 aa, Artificial Sequence
  Antibody C59 VH CDR2 (Chothia)
SKHG

SEQ ID NO: 67 - protein, 6 aa, Artificial Sequence
  Antibody C59 VH CDR3 (Chothia)
GRSAMD

SEQ ID NO: 68 - protein, 7 aa, Artificial Sequence
  Antibody C59 VL CDR1 (Chothia)
SQSVSSA

SEQ ID NO: 69 - protein, 6 aa, Artificial Sequence
  Antibody C59 VL CDR3 (Chothia)
RTTSLL

SEQ ID NO: 70 - protein, 6 aa, Artificial Sequence
  Antibody C59 VH CDR1 (Contact)
TSYYIH

SEQ ID NO: 71 - protein, 13 aa, Artificial Sequence
  Antibody C59 VH CDR2 (Contact)
WVAYIDSKHGTTQ

SEQ ID NO: 72 - protein, 9 aa, Artificial Sequence
  Antibody C59 VH CDR3 (Contact)
ARGGRSAMD

SEQ ID NO: 73 - protein, 7 aa, Artificial Sequence
  Antibody C59 VL CDR1 (Contact)
SSAVAWY

SEQ ID NO: 74 - protein, 10 aa, Artificial Sequence
  Antibody C59 VL CDR2 (Contact)
LLIYSASSLY

SEQ ID NO: 75 - protein, 8 aa, Artificial Sequence
  Antibody C59 VL CDR3 (Contact)
QQRTTSLL

SEQ ID NO: 76 - protein, 10 aa, Artificial Sequence
  Antibody C59 VH CDR2 (AbM)
YIDSKHGTTQ

SEQ ID NO: 77 - protein, 114 aa, Artificial Sequence
  Antibody C59 VH Sequence
EVQLVESGGGLVQPGGSLRLSCAASGFTFTSYYIHWVRQAPGKGLEWVAYIDSKHGTTQYADSVKGRFTISADTSKNTAYLQMNSLRAEDTAVYYCARGGRSAMDYWGQGTLVT

SEQ ID NO: 78 - protein, 107 aa, Artificial Sequence
  Antibody C59 VL Sequence
DIQMTQSPSSLSASVGDRVTITCRASQSVSSAVAWYQQKPGKAPKLLIYSASSLYSGVPSRFSGSRSGTDFTLTISSLQPEDFATYYCQQRTTSLLTFGQGTKVEIK

SEQ ID NO: 79 - protein, 323 aa, Homo sapiens
  Full-length amino acid sequence of human CD47
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 80 - protein, 504 aa, Homo sapiens
  Full-length amino acid sequence of human SIRPa
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 81 - protein, 222 aa, Artificial Sequence
  Exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 222 aa, Artificial Sequence
  Exemplary variant Fc region (silent Fc) sequence
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 10 aa, Artificial Sequence
  CD47 VH CDR1
  misc_feature (5)..(6): Xaa can be any naturally occurring amino acid
GFTFXXYYIH

SEQ ID NO: 84 - protein, 17 aa, Artificial Sequence
  CD47 VH CDR2
  misc_feature (1)..(1): Xaa can be any naturally occurring amino acid
  misc_feature (4)..(8): Xaa can be any naturally occurring amino acid
  misc_feature (10)..(10): Xaa can be any naturally occurring amino acid
XIDXXXXXTXYADSVKG

SEQ ID NO: 85 - protein, 8 aa, Artificial Sequence
  CD47 VH CDR3
  misc_feature (3)..(4): Xaa can be any naturally occurring amino acid
  misc_feature (6)..(6): Xaa can be any naturally occurring amino acid
GGXXAXDY

SEQ ID NO: 86 - protein, 11 aa, Artificial Sequence
  CD47 VL CDR1
RASQSVSSAVA

SEQ ID NO: 87 - protein, 7 aa, Artificial Sequence
  CD47 VL CDR2
SASSLYS

SEQ ID NO: 88 - protein, 9 aa, Artificial Sequence
  CD47 VL CDR3
  misc_feature (3)..(6): Xaa can be any naturally occurring amino acid
  misc_feature (8)..(8): Xaa can be any naturally occurring amino acid
QQXXXXLXT

SEQ ID NO: 89 - protein, 17 aa, Artificial Sequence
  CD47 VH CDR2
  MISC_FEATURE (1)..(1): X is a W, F or Y
  MISC_FEATURE (4)..(4): X is a P or S
  MISC_FEATURE (5)..(5): X is a Y or K
  MISC_FEATURE (6)..(6): X is a G, S or H
  MISC_FEATURE (7)..(7): X is a H or G
  MISC_FEATURE (8)..(8): X is a S or T
  MISC_FEATURE (10)..(10): X is a T, E or Q
XIDXXXXXTXYADSVKG

SEQ ID NO: 90 - protein, 8 aa, Artificial Sequence
  CD47 VH CDR3
  MISC_FEATURE (3)..(3): X is a R or L
  MISC_FEATURE (4)..(4): X is a G, Y or S
  MISC_FEATURE (6)..(6): X is a M or L
GGXXAXDY

SEQ ID NO: 91 - protein, 9 aa, Artificial Sequence
  CD47 VL CDR3
  MISC_FEATURE (3)..(3): X is a R or G
  MISC_FEATURE (4)..(4): X is a Y, R or T
  MISC_FEATURE (5)..(5): X is a S or T
  MISC_FEATURE (6)..(6): X is a S or D
  MISC_FEATURE (8)..(8): X is a L or R
QQXXXXLXT

SEQ ID NO: 92 - protein, 226 aa, Artificial Sequence
  Phage display heavy chain
  misc_feature (31)..(35): Xaa can be any naturally occurring amino acid
  misc_feature (50)..(66): Xaa can be any naturally occurring amino acid
  misc_feature (99)..(112): Xaa can be any naturally occurring amino acid
EVQLVESGGGLVQPGGSLRLSCAASGFTFSXXXXXWVRQAPGKGLEWVAXXXXXXXXXXXXXXXXXRFTISADTSKNTAYLQMNSLRAEDTAVYYCARXXXXXXXXXXXXXXWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 93 - protein, 214 aa, Artificial Sequence
  Phage display light chain
  misc_feature (89)..(97): Xaa can be any naturally occurring amino acid
DIQMTQSPSSLSASVGDRVTITCRASQSVSSAVAWYQQKPGKAPKLLIYSASSLYSGVPSRFSGSRSGTDFTLTISSLQPEDFATYYCXXXXXXXXXFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 330 aa, Artificial Sequence [sequence omitted]

SEQ ID NO: 95 - protein, 107 aa, Artificial Sequence
  Full length IgGs, the light chain constant region
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 330 aa, Artificial Sequence [sequence omitted]

SEQ ID NO: 97 - protein, 107 aa, Artificial Sequence; identical to SEQ ID NO: 95
  Exemplary light chain (e.g., for pairing with an IgG heavy chain) comprises any VL domain

SEQ ID NO: 98 - protein, 330 aa, Artificial Sequence
  Exemplary IgG heavy chain comprises any VH domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALKAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 35 aa, Artificial Sequence
  VH consensus sequence
  MISC_FEATURE (5)..(5): X is a S or T
  MISC_FEATURE (6)..(6): X is a Y or S
  MISC_FEATURE (11)..(11): X is a W, F or Y
  MISC_FEATURE (14)..(14): X is a P or S
  MISC_FEATURE (15)..(15): X is a Y or K
  MISC_FEATURE (16)..(16): X is a G, S or H
  MISC_FEATURE (17)..(17): X is a H or G
  MISC_FEATURE (18)..(18): X is a S or T
  MISC_FEATURE (20)..(20): X is a T, E or Q
  MISC_FEATURE (30)..(30): X is a R or L
  MISC_FEATURE (31)..(31): X is a G, Y or S
  MISC_FEATURE (33)..(33): X is a M or L
GFTFXXYYIHXIDXXXXXTXYADSVKGGGXXAXDY

SEQ ID NO: 100 - protein, 27 aa, Artificial Sequence
  VL consensus sequence
  MISC_FEATURE (21)..(21): X is a R or G
  MISC_FEATURE (22)..(22): X is a Y, R or T
  MISC_FEATURE (23)..(23): X is a S or T
  MISC_FEATURE (24)..(24): X is a S or D
  MISC_FEATURE (26)..(26): X is a L or R
RASQSVSSAVASASSLYSQQXXXXLXT

SEQ ID NO: 101 - protein, 10 aa, Artificial Sequence
  CD47 VH CDR1
  MISC_FEATURE (5)..(5): X is a S or T
  MISC_FEATURE (6)..(6): X is a Y or S
GFTFXXYYIH